(19) United States
(12) Patent Application Publication
(10) Pub. No.: US 2024/0392383 A1
(43) Pub. Date: Nov. 28, 2024

(54) Cancer biomarkers for susceptibility to treatment by almitrine

(71) Applicant: Michael David Forrest, Poole (GB)

(72) Inventor: Michael David Forrest, Poole (GB)

(21) Appl. No.: 18/693,873

(22) PCT Filed: Sep. 22, 2021
(86) PCT No.: PCT/IB2021/058639

§ 371 Date: Mar. 20, 2024

(51) Int. Cl. C12Q 1/6886; A61K 31/53

(52) U.S. Cl. CPC C12Q 1/6886; A61K 31/53; C12Q 2600/106; C12Q 2600/158

(57) ABSTRACT

Biomarkers of cancer susceptibility/resistance to almitrine treatment are disclosed.

### BACKGROUND

In previous patent applications (e.g. WO2019/012149A1, which published in the USA as US20200306253A1; a later filing of mine, with more content, is AU2019208238A1; all these filings are herein incorporated in their entirety by reference; if anything is unclear or ambiguous in the present text, [for non-limiting example if a term is undefined], guidance can be found in one or more of these published applications) I have reported how almitrine dimesylate has greater anti-cancer activity than many present cancer drugs (e.g. carboplatin, one of the most used) in standardized pre-clinical NCI-60 testing at the National Cancer Institute (USA). Yet almitrine dimesylate has less, close to no, side-effects in humans. Greater anti-cancer activity, less side-effects. This combination meets an unmet medical need.

### SUMMARY

Many cancer drugs cause horrific side-effects. Lethal in some cases. Nearly universally harming quality of life, without necessarily extending it by much. By contrast, almitrine dimesylate, which has been taken for decades in humans, and for millions of patient months [1], has no side-effects in nearly every patient that takes it daily for <11 months. And only causes side-effects in a very small minority of the many patients that take it daily for >11 months to years [2]. Wherein these side-effects, if they do occur, are relatively minor, and distinctly reversible (reversible upon stopping the course in nearly all cases [2]), compared to present cancer drugs (e.g. refer chemotherapy induced peripheral neuropathy, CIPN, often permanent). So, almitrine (e.g. almitrine dimesylate) can be prescribed to patients more freely than a typical cancer drug, because it is not likely to harm, especially when used short-term, and/or in cycles, as most cancer drugs are.

Almitrine is especially useful, meeting immense unmet medical need, for the significant proportion of patients that withdraw consent to cancer treatment because of side-effects. Either because they are absolutely intolerable for the patient. Or the patient doesn't consider them worth the predicted increase in life expectancy, which can be short. Moreover, almitrine is useful in cases that the cancer patient is elderly/frail, for (non-limiting) example over 60 years old, wherein such patients often can't physically withstand one or more of chemo-, radio-, immuno-therapy, surgery/transplant etc.

Because present cancer drugs are so harmful, there is a lot of research interest in trying to discover cancer biomarkers, which can predict if a cancer will respond to a particular cancer drug. With the aim that each cancer drug is only administered to cancer patients who stand a reasonable chance of being able to benefit from it. Because almitrine isn't typically harmful, the biomarker imperative isn't as much, but there is still much utility for biomarkers that can predict which cancers will best respond to almitrine treatment. For example, biomarkers can assist clinical trialling of almitrine vs. cancer. Wherein biomarker(s), predicting the cancers most susceptible to the anti-cancer activity of almitrine, enable those cancer patients likely to benefit most to be admitted to a clinical trial(s). Conferring a stronger clinical signal to the FDA/EMA or equivalent. Herein biomarkers, methods and kits are disclosed that can predict a cancer's susceptibility to the anti-cancer activity of almitrine (e.g. almitrine in the form of almitrine dimesylate). Excitingly, cancer susceptibility to almitrine dimesylate correlates with biomarkers that correlate with, and drive, resistance to many present cancer drugs, and poor prognosis. Thence, almitrine disproportionally targets at least some of the most dangerous cancers.

### The Broader Teaching

Componentry to this disclosure is each method I used to find the biomarkers of cancer susceptibility/resistance to almitrine, as presented herein. Moreover, any one or more of these methods applied to a different compound/drug, or plurality thereof. For non-limiting example, a compound/drug that has undergone NCI-60 testing at the National Cancer Institute (NCI), and/or the same/similar method of testing elsewhere. For non-limiting example, a drug approved for at least one clinical use, optionally/preferably for anti-cancer use, by one or more of the Food and Drug Administration (FDA) in the USA, the European Medicines Agency (EMA), the Medicines and Healthcare products Regulatory Agency (MHRA) in the UK, the Pharmaceuticals and Medical Devices Agency (PMDA) in Japan, the National Medical Products Administration (NMPA) in China, and/or one or more similar regulatory authorities in another country/jurisdiction (preferably, but not restrictively, which has been tested in NCI-60 testing). For non-limiting example, a compound/drug in clinical trials for cancer, or a compound/drug that has failed one or more clinical trials for cancer. And the biomarker(s) found by one or more of the methods herein are componentry to this present disclosure. Also componentry to this disclosure are method variants/truncations/extensions/equivalents, which will be apparent to one of the art, now they have this disclosure in hand. And the biomarker(s) found by such a method equivalent(s)/variant(s).

A componentry method of this disclosure is to enter the CAS number (or name) of an approved/licensed/candidate/failed candidate cancer drug (e.g. approved by the FDA and/or EMA, and/or equivalent/similar regulatory body in another country/jurisdiction) into the text box of https://dtp.cancer.gov/dtpstandard/dwindex/index.jsp (making the appropriate radio button selections, as clear to one of the art). This database is hereby incorporated in its entirety by reference; indeed, all databases referred to herein are incorporated herein in their entirety by reference. Then, if the drug is present in this database, noting its “NSC number”, findable in its database entry. Then using this NSC number with NCI COMPARE, available at http://dtp.cancer.gov/public_compare/ (or, which might be found in the future at, https://nci60.cancer.gov/publiccompare/), introduced at https://dtp.cancer.gov/databases_tools/compare.htm, and selecting its settings, and analysing its output, optionally corroborating (e.g. by using one or more of the corroborating methods herein, e.g. by using the CellMiner database [3-4]), by at least one method herein, and/or by a equivalent(s)/variant(s) thereof, to retrieve a biomarker(s) for cancer susceptibility/resistance to that cancer drug. This biomarker(s) can then be used in a further method(s) herein, and/or in a method(s) of the art for using a biomarker(s). As illustrated herein, taught by way of example, for almitrine dimesylate.

This method can be performed for a number of approved/licensed/candidate/failed candidate cancer drugs, optionally by selecting one or more cancer drugs from a publicly available list(s) of cancer drugs (e.g. as found by using the Google search engine, non-limiting e.g. https://www.cancer.gov/about-cancer/treatment/drugs, https://www.cancerresearchuk.org/about-cancer/cancer-in-general/treatment/cancer/cancer-drugs/drugs, https://en.wikipedia.org/wiki/List_of_chemotherapeutic_agents, https://en.wikipedia.org/wiki/List_of_antineoplastic_agents) and/or, in a preferred embodiment, listed in the paper (and/or in one or more of its supplementary tables): Holbeck S L, Collins J M, Doroshow J H (2010) Analysis of Food and Drug Administration-approved anticancer agents in the NCI60 panel of human tumor cell lines. Molecular cancer therapeutics. 9(5):1451-60.

A componentry method of this disclosure is to enter the name or, more preferably, the NSC number (NSC numbers are presented in brackets after each cancer drug name) of one of the following cancer drugs into the appropriate text box of https://dtp.cancer.gov/public_compare/, and subsequently using a method(s) herein (e.g. as illustrated, teaching by way of example, for almitrine dimesylate), and/or an equivalent/variant(s) thereof, to retrieve a biomarker(s) for cancer susceptibility/resistance to that cancer drug, wherein in some embodiments this is done for a plurality of these cancer drugs, and in some embodiments it is done for all of these cancer drugs:
Bortezomib (681239), Dasatinib (732517), Erlotinib (718781), Everolimus (733504), Gefitinib (715055), Imatinib (743414), Lapatinib (745750), Nilotinib (747599), Sorafenib (747971), Sunitinib (750690), Temsirolimus (683864), Actinomycin D (3053), BCNU (Carmustine) (409962), Bendamustine (138783), Bleomycin (125066), Busulfan (750), Carboplatin (241240), CCNU (Lomustine) (79037), Chlorambucil (3088), Cisplatin (119875), Cyclophosphamide (26271), Dacarbazine (45388), Hexamethylmelamine (13875), Ifosfamide (109724), Melphalan (8806), Methoxsalen (45923), Mitomycin C (26980), Mithramycin (24559), Mustine/Nitrogen mustard (762), Oxaliplatin (266046), Pipobroman (25154), Procarbazine (77213), Quinacrine (14229), Streptozotocin (85998), Temozolomide (362856), ThioTEPA (6396), Triethylenemelamine (9706), Uracil mustard (34462), Docetaxel (628503), Ixabepilone (747973), Paclitaxel (125973), Vinblastine (49842), Vincristine (67574), Vinorelbine (608210), Daunorubicin (82151), Doxorubicin (123127), Epirubicin (256942), Etoposide (141540), Idarubicin (256439), Irinotecan (616348), Mitoxantrone (301739), Teniposide (122819), Topotecan (609699), Valrubicin (246131), 5-azacytidine (102816), 5-fluorouracil (19893), 6-Mercaptopurine (755), Allopurinol (1390), Calcium leucovorin (3590), Capcitabine (712807), Cladribine (105014), Clofarabine (606869), Cytarabine (63878), Decitabine (127716), Floxuridine (27640), Fludarabine (312887), Gemcitabine (613327), Hydroxyurea (32065), Methotrexate (740), Nelarabine (686673), Pemetrexed (698037), Pentostatin (218321), Thioguanine (752), Anastrozole (719344), Delta-1-testololactone (23759), Dimethyltestosterone (88536), Dromostanolone propionate (12198), Estramustine (702294), Ethinyl estradiol (10973), Exemestane (713563), Fulvestrant (719276), Letrozole (719345), Megestrol acetate (71423), Mitotane (38721), Naldrolone (23162), Raloxifene (747974), Tamoxifen (180973), Toremifene (613680), Amifostine (296961), Aminolevulinic acid (18509), Arsenic trioxide (706363), Celecoxib (719627), Dexrazoxane (169780), Imiquimod (369100), Lenalidomide (747972), Mitomycin (134727), Mitomycin (26980), Mitomycin (755880), BEN (281612), Uracil mustard (34462), Nitrogen mustard (757087), Melphalan (757098), Nitrogen mustard (762), 8-Chloro-adenosine (354258), Simvastatin (633782), Itraconazole (754771), Buthionine sulphoximine (326231), Simvastatin (758706), Hydroxyurea (32065), Curcumin (32982), Imexon (714597), Arsenic trioxide (706363), Arsenic trioxide (759274), Salinomycin (751548), Irofulven (683863), Obatoclax (744912), 1^{st} Intermediate to TDP 665759 (756877), Bafetinib (773263), Dasatinib (759877), Dasatinib (732517), BN-2629 (694501), Pyrazoloacridine (366140), Pyrazoloacridine (627168), Bleomycin (758612), Aminoflavone (686288), AFP464 (710464), Pralatrexate (754230), Azacitidine (758186), Triapine (663249), Cordycepin (63984), Cytarabine (287459), Fenretinide (374551), Clofarabine (759857), Fenretinide (760419), Fludarabine (118218), Fludarabine (124463), Raltitrexed (639186), 6-Mercaptopurine (752), 6-Mercaptopurine (757348), 6-Mercaptopurine (759614), Fluorouracil (19893), Fluorouracil (757036), Floxuridine (758230), Pelitrexol (767745), Vorinostat (759852), Belinostat (758774), Vorinostat (701852), Depsipeptide (630176), kahalide f (668814), Belinostat (726630), SR16157 (732011), Dexamethasone Decadron (34521), Dromostanolone Propionate (12198), Abiraterone (749226), AT-13387 (749712), By-Product of CUDC-305 (761390), geldanamycin analog (255109), Alvespimycin (707545), Lificguat (728165), LOR-253 (763832), 3-Bromopyruvate acid (62343), Dimethylaminoparthenolide (734325), Tyrothricin (757363), Hydrastinine HCl (755892), Digoxin (756738), Acetalax (117186), Acetalax (59687), bisacodyl (614826), Pimozide (170984), Pipamperone (759178), Fluphenazine (92339), Olaparib (747856), Olaparib (753686), okadaic acid (677083), LDK-378 (777193), Dabrafenib (764134), AZD-9291 (779217), Cobimetinib isomer1 (768068), LY-294002 (697286), Staurosporine (618487), Midostaurin (656576), RH1 (697726), Elesclomol (174939), PX-316 (710297), Triciribine phosphate (280594), 7-Hydroxystaurosporine (638850), 7-Hydroxystaurosporine (638646), Seliciclib (701554), Palbociclib (758247), PD-98059 (679828), Trametinib (758246), Rapamycin (606698), Rapamycin (226080), Selumetinib (741078), Hypothemycin (354462), AP-26113 (761191), Vemurafenib (761431), Ibrutinib (761910), Ruxolitinib (763371), Crizotinib (756645), Cabozantinib (757436), Cabozantinib (761068), Pazopanib (737754), Pazopanib (752782), Lenvatinib (755980), Ixazomib citrate (758254), Carfilzomib (758252), Sonidegib (761385), LMP-400 (724998), 7-Ethyl-10-hydroxycamptothecin (673596), Karenitecin (710270), LMP776 (725776), Irinotecan (728073), Topotecan (759263), Irinotecan (759878), 7-Tert-butyl-10-hydroxycamptothecin (708298), Lapachone (629749), Etoposide (757804), Teniposide (758255), Daunorubicin (756717), Teniposide (758667), Doxorubicin (759155), Epirubicin (759195), Mitoxantrone (279836), Mitoxantrone (301739), Daunorubicin (83142), Elliptinium Acetate (264137), Batracylin (320846), Eribulin mesilate (707389), Benzimate (109874), Dolastatin 10 (376128), Vincristine (759174), Vinorelbine (760087), Vinblastine (757384), Vinblastine (90636), Docetaxel (759850), Paclitaxel (758645), Tfdu (75520), 5-fluoro deoxy uridine 10mer (697912), Enzalutamide (766085), Enzalutamide (755605), Osimertinib (779217), Ivosidenib (789102), Vorasidenib (803600), Enasidenib (788120), Pomalidomide (775351), Thalidomide (66847), Venclexta (766270), Darolutamide (825331), Vindesine sulfate (781619), Bicalutamide (759816), Abiraterone acetate (749227), Prednisolone (9120), Risperidone (786035); Pixantrone (CAS number: 144510-96-3).

Herein, by disclosing the database (herein incorporated in its entirety by reference, especially the database entries of the aforementioned NSC numbers), the cancer drug(s), and a method(s) to retrieve a biomarker(s) for cancer susceptibility/resistance (to each specified cancer drug) from the database, the biomarker(s) has been disclosed herein (incorporated herein by reference). In a very efficient, concise manner. This biomarker(s) can then be used in a further method(s) herein, and/or in a method(s) of the art for using a biomarker(s).

With the method(s) disclosed herein in hand, retrieving such a biomarker(s) is routine. By disclosing the method(s), where and how to apply the method(s), I have disclosed the biomarker(s). The number of cancer drugs listed here is relatively small, and one of the art can readily use the teaching of this disclosure to retrieve zero or more biomarkers for each of these named/specified cancer drugs, meaning any such biomarker(s) is in turn componentry to this disclosure, as is its use in a method(s) herein, and/or in a method(s) of the art for using a biomarker(s).

If any of the above internet links become non-functional, then the new, updated links or equivalents can be found by contacting the National Cancer Institute (NCI, USA), especially its Developmental Therapeutics Program (DTP) section. This contact can include going to the NCI to enquire. If there is any difficulty in navigating the internet pages at the NCI, again guidance is available from the NCI team, e.g. from Dr. Mark W. Kunkel there (Email: kunkelm@mail.nih.gov). Note that a symbol/icon used for COMPARE, at some points on the NCI website, is a circle with a capital C inside of it. Downloading, for offline use, any database mentioned herein is contemplated. If for some reason a method herein isn't possible for one drug mentioned herein, try with a different drug(s) herein.

If/when a cancer biomarker(s) that is already known to apply for the drug (it is part of the state of the art) is found by this method, in some embodiments it is dismissed. Preferably substituted by a biomarker(s) that is not part of the state of the art.

Particularly contemplated is using a biomarker(s), found by a method(s) herein, to select a cancer patient(s) for a candidate cancer drug trial. And/or to (optionally retrospectively) analyse a candidate cancer drug trial (possibly assessing whether the drug was more active for any sub-population(s) of subjects [their cancers], as identified by one or more biomarkers). And/or to select which cancer drug(s) to administer to a subject with cancer. In some embodiments, if a subject's cancer is predicted to be susceptible/responsive to a compound(s)/drug(s), by one or more biomarkers acquired by a method(s) herein, then a therapeutically effective amount of this compound(s)/drug(s) is administered to the subject. In some embodiments, if a subject's cancer is predicted to be resistant to a compound(s)/drug(s), by one or more biomarkers acquired by a method(s) herein, then this compound(s)/drug(s) is not administered to the subject, optionally wherein a different compound(s)/drug(s) is administered to the subject instead, optionally a compound(s)/drug(s) that the subject's cancer is predicted to be susceptible/responsive to by one or more biomarkers acquired by a method(s) herein.

A preferred cancer drug(s) to retrieve a biomarker(s) for and use, using a method(s) of this disclosure, is a cancer drug with a large number of USD $sales in the preceding year. For example, of the order of USD $billions. For non-limiting example, one or more of (NSC numbers in brackets) Lenalidomide (747972), Ibrutinib (761910), Palbociclib (758247), Enzalutamide (766085; 755605), Osimertinib (779217). Note that Biologics (e.g. antibody based drugs, e.g. checkpoint inhibitors thereof) are contemplated. Including immunotherapies. Including (without limitation) Nivolumab (Opdivo), Pembrolizumab (Keytruda), Ipilimumab (Yervoy), Atezolizumab, Avelumab, Durvalumab.

A preferred drug(s) to retrieve a biomarker(s) for and use, using a method(s) of this disclosure, is a candidate cancer drug (or failed candidate thereof) developed/owned by a multinational (and/or publically listed) pharmaceutical company. Optionally one of those listed in a list of 100 (or 20, or 10) pharmaceutical companies with the greatest USD$ annual sales in the preceding year. For example, Servier.

### DETAILED DESCRIPTION OF THE INVENTION

In clinical use, almitrine is often administered as almitrine dimesylate. But the teaching herein is not restricted to that form, and other salts of almitrine, and the administration of almitrine without a salt, are also contemplated. Indeed, without restriction, any clinically used form of almitrine (and/or form disclosed in WO2019/012149A1) is contemplated. Herein a reference to almitrine, or a salt thereof, is, in alternative embodiments, a reference to a different almitrine containing composition, preferably a pharmaceutical composition thereof. Some prototypical, non-limiting, almitrine containing compositions are specified in WO2019/012149A1.

If a term/component is undefined or unclear/ambiguous herein, please refer to WO2019/012149A1 or AU2019208238A1 for clarification/specification, which are both herein incorporated in their entirety by reference. For example, the bounds of a “subject” is specified therein. Routes of administration, some (without restriction) contemplated cancer types etc. are therein and thence are herein by incorporation by reference. In some embodiments, “effective amount” herein is substituted with “therapeutically effective amount”. Where a mean or average is referred to herein, this can be the arithmetic mean, or (in alternative embodiments) some other mean (e.g. logarithmic, e.g. harmonic), or be the median instead. At points herein that a Pearson correlation coefficient is referred to, or used, in alternative embodiments a different correlation measure of the art is used, e.g. Spearman's rank correlation coefficient. If there is any contradiction of definition(s) in this disclosure, all the given definitions are valid but for different embodiments of the disclosure.

### Primer to Following Method: Transcripts Instead of Genes

A single gene typically produces multiple transcripts. In humans, the average is 15.2 different transcripts per gene [5]. The method described in the section hereafter operates at the gene level, by assuming that different transcript sequences from a single gene correlate, or don't correlate, similarly to a drug(s) susceptibility. But this assumption can be misplaced. So, also componentry to this disclosure is to operate at the transcript rather than the gene level: to consider different sequence transcripts from the same gene independently, incorporating the possibility that a sequence transcript(s) of a gene can correlate with a drug(s) susceptibility/resistance, whilst a different sequence transcript(s) from the same gene doesn't, or doesn't to the same degree. Herein, wherever gene expression is referred to, in alternative embodiments, a transcript sequence(s) expression, independently of a different sequence transcript(s) from the same gene, is being referred to instead. So, to predict a cancer's susceptibility to a drug(s), instead of looking at the gene expression level, wherein any one or more of a gene's different sequence transcripts is considered, possibly wherein their expression is averaged, the expression of one or more specific sequence transcripts from a gene(s) is considered, the expression of which correlates with drug(s) susceptibility or resistance.

### Method

Distinct from above, for almitrine dimesylate I used a different web link to access NCI COMPARE: https://dtp.cancer.gov/private_compare/login.xhtml. Where this link is only for those with a private account. Because, distinctly, the NCI-60 data for almitrine dimesylate is not yet public at the NCI. And is only accessible via my private, password protected account.

GI50 is compound concentration that causes 50% growth inhibition of a cell line relative to no-drug control. Cancer gene expression correlations to almitrine dimesylate susceptibility were found using [NCI 5-dose almitrine dimesylate GI50 anti-cancer data] with the NCI COMPARE algorithm [6-7]. NCI COMPARE outputs the Pearson correlation coefficient (R) between each gene expression and −log 10(GI50). So, NOT GI50! Thence, a greater positive correlation=higher gene expression=lower GI50, i.e. greater anti-cancer drug potency. Six independent gene expression data sets were searched, wherein these were from a most favoured/trusted collection at the NCI, called “MOLTID_GC_SERIES_MICROARRAY_ALL” in the NCI COMPARE system. This delimited collection excludes the other gene expression data sets available in NCI COMPARE that aren't determined as equivalently/sufficiently reliable by the NCI staff. This concurrent searching in six independent trusted gene expression data sets enables corroboration; is a correlation observed with same sign, and at notable magnitude, in more than one trusted gene expression data set? Moreover, because some genes can produce more than one transcript sequence [5], each gene expression data set can include more than one transcript sequence for each gene. Indeed, these gene expression data sets are better thought of as transcript expression data sets, because a single gene, in many—but not all—cases, can produce multiple different sequence transcripts. Wherein the expression of each can be independently (or non-independently, depending on probe design) reported, assuming each was known to exist (or predicted to exist) at the time of the microarray chip design, and a probe for each was selected for incorporation on the constrained space of the chip. In cases that multiple different sequence transcripts/probes from a single gene correlate with susceptibility to almitrine dimesylate, this is stronger affirmation that this gene expression correlates with susceptibility/resistance to almitrine dimesylate. Indeed, the more times that a gene expression is observed to appreciably correlate, with same sign, with susceptibility to almitrine dimesylate, within a single gene expression data set, and/or across different gene expression data sets, the more conviction we can have that the correlation is valid. Componentry to this disclosure is to rank/order/select gene expression correlation(s) to a drug(s) susceptibility/resistance, for reliability/robustness/conviction, by the number of times that each correlation is observed above a selected threshold (e.g. Pearson) correlation coefficient value (|R|). Using this ranking method, potentially more conviction can be drawn for genes with a greater number of different sequence transcripts/probes in the gene expression database(s) used, biasing towards these. This bias can be removed by dividing each gene value (the number of times that a correlation, above a selected value of |R|, is observed for that gene), by the number of probes for that gene. Alternatively, this bias can be removed by only considering a single transcript/probe sequence per gene, per gene expression database, preferably wherein the transcript/probe sequence with the highest correlation to the drug(s) susceptibility/resistance is selected. Another source of bias can come if not all the same genes are necessarily componentry to all the gene expression databases (e.g. 2 of the 6 gene expression databases in the selected NCI gene expression database set are much smaller than the other 4, as shown in a table later): a given gene may be present in one or more of the gene expression databases searched, wherein a strong correlation to a drug sensitivity might be detected, but absent from another gene expression database(s), and so using this ranking method by number of occurrences observed above correlation threshold, the relative importance of this gene expression correlation, as compared to other genes that are present in all the gene expression databases considered, might potentially be underestimated. However, although this method, uncontrolled, might conceivably, for some use scenarios, underestimate the relative importance of some gene expression correlations as compared to others, the most highly ranked gene expression correlations by this method, especially if a high threshold correlation value is selected, are likely to be absolutely robust and reliable. This is very valuable.

In the table below, the six gene expression data sets that were used within NCI COMPARE, are each allocated a serial code that will be used to refer to each hereafter, with their gene numbers estimated, enabling a multiple comparisons correction for each:

USING NCI COMPARE

| | | Transcript | Gene | Bonferroni corrected |
|---|---|---|---|---|
| Gene expression data set (“Set”) | Serial | number | number | p-value threshold |
| MOLTID_GC_SERIES_MICROARRAY_MOCK_TX_24H | MTX | 44942 | 16775 | 0.05/16775 = 0.00000298062 = 0.000003 |
| MOLTID_GC_SERIES_MICROARRAY_GENELOGIC_U95 | 095 | 63108 | 16211 | 0.05/16211 = 0.00000308432 = 0.000003 |
| MOLTID_GC_SERIES_MICROARRAY_GENELOGIC_U133 | U133 | 44928 | 16788 | 0.05/16788 = 0.00000297831 = 0.000003 |
| MOLTID_GC_SERIES_MICROARRAY_GENELOGIC_U133PLUS2 | U133+ | 54675 | 16803 | 0.05/16803 = 0.00000297565 = 0.000003 |
| MOLTID_GC_SERIES_MICROARRAY_NOVARTIS | NTS | 12626 | 7516 | 0.05/7516 = 0.00000665247 = 0.000007 |
| MOLTID_GC_SERIES_MICROARRAY_STANFORD | STD | 9706 | 6227 | 0.05/6227 = 0.00000802954 = 0.000008 |

Transcript number includes transcripts from genes, including different sequence transcripts from same genes, pseudogenes and open reading frames. These transcript numbers were sourced from Dr. Mark Kunkel, point of contact for COMPARE at the NCI. Gene numbers are only estimates. Estimated by only counting one transcript with each gene name (“Genecard”), and by dismissing transcripts for which their “Genecard” is “null” or starts with “Loc” or “ORF”.

Correlations first found using NCI COMPARE, with the aforementioned method, were then further corroborated using cancer gene expression data sourced from the CellMiner database [3-4]. Indeed, the following NCI 1-dose and 5-dose anti-cancer data for almitrine dimesylate, with serial codes given that will be used to refer to each hereafter, was compared with cancer gene expression data sourced from CellMiner:

| Almitrine dimesylate anti-cancer | |
|---|---|
| data set (“Set”) from NCI | Serial |
| NCI 5-dose protocol (GI50) | 5-dose(GI50) |
| Growth inhibition (%), NCI 5-dose protocol (@10 μM) | 5-dose(10 μM) |
| Growth inhibition (%), NCI 1-dose protocol (@10 μM) | 1-dose(10 μM) |
| Mean of 1- and 5-dose growth inhibition (%) at 10 μM | Mean[1-\|5-] |
| | dose(10 μM) |

The CellMiner database includes five independent Human Genome gene expression data sets {accession numbers: GSE5949, GSE5720, GSE32474, GSE29682, GSE29288}, each of which can have multiple different transcript sequence probes for a single gene, wherein the mean expression for each gene was taken: within and across the different databases, which is distinct from NCI COMPARE where all expressions of different sequence transcripts for a gene were kept distinct without any averaging, and where there was no averaging across gene expression data sets, each was kept distinct. Although actually only four of these gene expression data sets in CellMiner were used to generate the mean for each gene expression: the single gene expression data set that used an Agilent instead of an Affymetrix microarray chip (GSE29288) was excluded from the mean because, to quote the CellMiner documentation: “Agilent probe(s) values are removed from cell line average intensity calculation due to normalization inconsistency with Affymetrix probesets”. Raw (log 2), rather than z scored, gene expression data was used (using z scored data would have permitted the Agilent probe data to be used also, since according to CellMiner documentation, z scoring makes Affymetrix and Agilent probe data interoperable, but raw data was favoured anyhow. Some exploratory testing using the z scored, Agilent incorporating, data instead of raw data showed essentially equivalent results for the data fraction looked at). So, the data set sourced from an Agilent microarray chip was not incorporated in the mean, but was instead used by itself separately for an independent verification. With much less data, the Agilent set is much less reliable than the mean of the 4 Affymetrix microarray data sets, but it is a nice independent adjudicator. This Agilent data set is not one of the six gene expression data sets at NCI COMPARE. By contrast, there is some overlap in the 4 CellMiner gene expression databases that were used for the mean and the 6 that were used with NCI COMPARE (some might be the same). However, at least one of these data sets is not found in NCI COMPARE (its accession number: GSE29682). Also, distinctly with the 4 Affymetrix microarray data sets in CellMiner, the mean expression for each gene was taken: averaging the expression of all the different sequence transcripts, across all the 4 used Affymetrix microarray data sets, for each gene. Moreover, this mean is very heavily weighted to a database that CellMiner has, and NCI COMPARE definitely doesn't, Accession number: GSE29682, because it tends to have many more probes for each gene than any other data set, indeed often more than all the others combined, each of which is an additional data point in the mean for each gene. This is because the data set with Accession number, GSE29682, was created using the Affymetrix Human Exon 1.0 ST Array, which has 4 probes per exon on average, and thence the number of probes for any given gene tends to scale with the number of exons it has, where the average gene has 10 exons and so there are, on average, 40 probes per gene on this chip. Thus, there is definite margin for the gene expression correlations drawn from CellMiner to diverge from NCI COMPARE, and thence their non-divergence, and agreement, is a strong, valuable confirmatory signal. Given its inclusion of GSE29682, which is the most recent and advanced gene expression data set for the NCI-60 cancer cell lines, sourced by utilising the most recent and advanced microarray chip, the Affymetrix Human Exon 1.0 ST Array, the CellMiner data is to be particularly prized. An optional additional step could conceivably make it marginally better, but this wasn't implemented (“juice not worth the squeeze”—would make no tangible difference in most cases), wherein, across the NCI-60 cancer cell lines, if an exon probe(s) expression in CellMiner doesn't appreciably correlate (Pearson, e.g. |R|>0.3) with the majority of the other exon probes for that same gene in CellMiner, it is cut from consideration and isn't used in the calculation of the mean expression for that gene.

To make a multiple comparisons correction the gene number in the gene expression database needs to be known. There are 25,722 transcripts (including genes, pseudogenes, and open reading frames) in the CellMiner database [8], wherein this transcript number can be used in lieu of gene number, which overcorrects.

Bonferroni corrected p-value threshold=0.05/25,722=0.00000194386=0.000002.

Šidák corrected p-value threshold=1−(1−0.05)\hat{ }(1/25,722)=0.00000199413=0.000002.

These are both overly conservative Multiple Comparisons corrections because a non-gene would never be selected, yet non-genes (e.g. pseudogenes) are included in the correction.

Furthermore, Bonferroni (or Šidák) is a very conservative correction method, erring to exclude false positives at the risk of introducing false negatives i.e. potentially missing true, rather than possibly reporting false, correlations.

Bonferroni corrected p-value threshold for 2 genes in combination=0.05/(25,722*2)=0.000000971930643=0.000001.

Bonferroni corrected p-value threshold for 9 genes in combination=0.05/(25,722*9)=0.000000215984587=0.0000002.

An alternative/supplement to a Multiple Comparisons correction, as utilized by this work, is to observe if a sizeable correlation (with same sign, + or −) is observed in independently sourced data sets: the more, the greater likelihood it is a true correlation.

To recap so far, correlations first found using NCI COMPARE, with the aforementioned method, were then externally corroborated using cancer gene expression data, produced by microarray chip technology, sourced from the CellMiner database [3-4]. To disclose the next step now: these correlations were then further corroborated by using RNA-seq gene expression data from the CellMiner database [9]. Such corroboration is partially hampered because the correlation between microarray and RNA-seq sourced gene expression data is not complete in CellMiner: Pearson correlation coefficient=0.64 [9]. However, good concordance was observed herein, generally. In some embodiments, if a subject's cancer's RNA-seq gene expression data is going to be a/the method used to select a subject(s) for almitrine dimesylate treatment, a gene(s) expression(s) that correlates in RNA-seq data to almitrine dimesylate susceptibility should be employed for (de-)selection. Gene isoforms are mRNAs that are produced from the same DNA locus by different transcription start and/or stop sites, splicing variation etc. The RNA-seq method permits individual reporting upon the expression of different isoforms of a gene(s) [9]. Herein, presenting as in CellMiner, isoform gene expressions are reported as log 2(FPKM+1), where FPKM is “Fragments Per Kilobase per Million reads” and the “composite” gene expression of all the isoforms of a gene, presenting as defined in CellMiner and as used herein, is, to illustrate by example, for IsoformX and IsoformY: log 2(1+((2\hat{ }(IsoformX)−1)+(2\hat{ }(IsoformY)−1))), where IsoformX=log 2(FPKM+1) and IsoformY=log 2(FPKM+1).

### Summary of Method Used:

Cancer gene expression correlations to almitrine susceptibility were first found using:
(1) NCI COMPARE (using 6 independent gene expression data sets, independently, which permits corroboration)
   Correlations Found were then Externally Corroborated Using:
(2) CellMiner (using mean data from 4 of its gene expression data sets, which were sourced using microarray chips made by Affymetrix).
(3) CellMiner (using mean data from its only gene expression data set that was sourced using a microarray chip made by Agilent).
(4) CellMiner (using its RNA-seq gene expression data set).

In alternative embodiments of the disclosure, this method, and/or component(s)/variant(s)/derivative(s) thereof, is utilized with a different drug(s). Optionally a candidate (or failed candidate) and/or approved (e.g. FDA/EMA/MHRA/PMDA/NMPA approved) anti-cancer drug(s), optionally one or more of the named/specified cancer drugs herein.

### Results

Assaying the gene expression of a cancer to predict its susceptibility to almitrine dimesylate treatment: the most predictive power, for least effort/complexity, is acquired by using [SCAF11-RAPH1], wherein a greater product predicts greater cancer susceptibility. Basis shown in FIG. 1A. More predictive power can be acquired by considering further genes in addition, as shown, for non-limiting example, by FIG. 1B. Of note, greater MYC gene expression predicts greater susceptibility to almitrine dimesylate, as shown in FIG. 1C, which suggests that almitrine dimesylate disproportionally harms the most dangerous cancers, most resistant to present treatments, with the worst prognosis.

This Results section presents results that build up to the most robust/favoured correlations, which are disclosed late in this section under the “RNA-seq data” heading, which presents RNA-seq alongside microarray data. And, in turn, the most favoured of those correlations are disclosed in FIG. 1 and its legend. So, a shortcut to the most favoured is to go directly there. However, further teaching and data will be acquired by reading this section fully, and reaching that section and its related figure in due time. Moreover, the equation for the Z parameter in FIG. 1B isn't necessarily the most optimal, because not all the good options were exhaustively searched, and a better equation(s) is likely possible drawing from the selected gene expression correlations presented in this Results section in its entirety. Which in turn is componentry to this disclosure. A general principle is that the predictive power of the equation, for predicting how susceptible/resistant a cancer is to almitrine dimesylate treatment, increases the more genes it includes with good correlation to almitrine dimesylate susceptibility/resistance. Without intent to limit, those that correlate with susceptibility are incorporated into the equation via addition, those that correlate with resistance via subtraction. However, note that adding genes with lower correlation can atrophy the predictive power of an equation that already utilizes genes with high correlation. So, a componentry strategy to this disclosure is to only include genes of reasonably high correlation, the higher the better, and the more genes of high correlation the better. Routine experimentation, as clear to someone of the art, with the gene correlations provided herein can converge upon the most optimal, or collection of most optimal, equations of Z.

Some selected Affymetrix microarray chip sourced results:

[symbol]

A comparator from other's work, giving perspective upon what a good correlation is: PTGR1 (alkenal/one oxidoreductase, AOR), which has been experimentally shown to bioactivate irofulven (hydroxymethylacylfulvene, HMF) into a potent anti-cancer form [10-11]: in NCI COMPARE, using NCI 5-dose testing data, PTGR1 expression correlates with susceptibility to the anti-cancer activity of irofulven: five correlations observed at >0.4, one of which is >0.5 at 0.523. To contrast, by example, in NCI COMPARE: SCAF11 expression correlation to anti-cancer activity of almitrine dimesylate: eight correlations observed at >0.4, one of which is >0.5 at 0.542.

In the gene lists below, following each gene name, in brackets, is how many times in NCI COMPARE's six independent gene expression data sets (some having multiple probes for some genes, typically because these genes produce more than one transcript sequence) that the gene expression correlation to almitrine dimesylate susceptibility is observed at: |R|>0.5, >0.4, >0.3 respectively. In each gene case below, the correlation is in the same direction (positive or negative) for all the observations. Greater faith can be entrusted in a correlation the more times it is observed, with same directionality. For some gene expressions, the number of observations of correlation to almitrine dimesylate susceptibility is large e.g. SCAF11. The ordering of the genes, as shown, ranks by number of correlation observations, with some intersecting consideration of amplitude of observed correlations also. Ranking (completely by, or in part by) gene expression correlations to a drug(s) activity: by number of times that this correlation is observed (with same directionality) is componentry to this disclosure, wherein, in a further embodiment(s), this ranking is used to identify which gene expression(s) to utilize in predicting a drug(s) activity, wherein, in a further embodiment(s), a cutoff is used, above which—above a number of observed times (above a specified |R| cutoff e.g. |R|>0.3)—gene expression correlations are included in the drug(s) activity prediction model, and at or below which they are excluded. In a componentry embodiment(s), when not all the observed correlations (e.g. above the cutoff e.g. |R|>0.3) for a gene expression are in the same direction (positive or negative) this gene is excluded from the prediction model. In a non-limiting embodiment(s) the cutoff is set at: observed at, or more than, two times (at |R|>0.3). The cutoff can be set higher for greater reliability (either by increasing the observed number requirement and/or increasing x in the |R|>x stipulation) and a smaller number of genes in the prediction model. Or the cutoff can be set lower. In an embodiment(s), this method is used with NCI COMPARE output data. In an embodiment(s) a correction(s) is applied if the same number of probes for each gene is not present in the database(s), wherein a proportionally lower observation cutoff is used for a gene with less probes. In an embodiment(s), and as a separate standalone method of the disclosure also (can be used with or without any other step herein), gene expressions found to be relevant are combined, using a method(s) as disclosed herein (refer to the disclosure section later with formulae for “GENE Q” and “GENE Y”), which can increase the predictive power above any individual gene expression used alone (e.g. refer FIG. 1). In an embodiment(s) the method, and/or component(s)/variant(s)/derivative(s)/equivalent(s) thereof (non-limiting e.g. as presented later), is used to produce an oral/audio/written/electronically encoded/computer medium report(s) upon whether a subject's cancer is likely to be susceptible/responsive/resistant to a drug(s) and/or the method, and/or component(s)/variant(s)/derivative(s)/equivalent(s) thereof (e.g. as presented later), is used to decide/determine (and/or as an input into deciding/determining) whether to administer an anti-cancer (optionally only candidate or failed candidate; optionally FDA/EMA/MHRA/PMDA/NMPA licensed) drug(s) to a subject, whereupon if/when the gene(s) expression of the subject's cancer predicts susceptibility to a drug(s) by this method, and/or component(s)/variant(s)/derivative(s)/equivalent(s) thereof, then the drug(s) is administered to the subject. In an embodiment(s), this method, and/or component(s)/variant(s)/derivative(s)/equivalent(s) thereof, is used for almitrine (and/or a pharmaceutically-acceptable salt, solvate, hydrate or prodrug thereof). Or a different named/specified cancer drug(s) herein.

### “GENE Q” Genes

whose greater mRNA expression in cancer is observed to correlate with greater cancer susceptibility to anti-cancer activity of almitrine dimesylate:
SCAF11 (1, 8, 11), SLC38A1 (0, 8, 10), USP36 (1, 6, 7), PGLS (0, 5, 8), CAMKK2 (0, 5, 5), DNAJC12 (0, 4, 9), TMPRSS3 (0, 4, 9), MYC (0, 4, 8), ZCCHC6 (0, 4, 7), CBLL1 (1, 4, 6), PCID2 (0, 4, 6), RNF144B (0, 4, 6), TRIM13 (0, 3, 6), DDX49 (0, 3, 5), MAU2 (0, 3, 5), SLC35E1 (0, 3, 5), ZNF766 (1, 3, 4), ZNF277 (0, 3, 4), ILF2 (0, 3, 4), HNRNPA1 (0, 3, 4), NPTX2 (0, 3, 4), NADSYN1 (0, 3, 4), HMX1 (0, 3, 4), MROH1 (0, 3, 4), DDX41 (0, 3, 4), NRBF2 (0, 3, 4), FABP6 (1, 3, 3), PTH2R (0, 3, 3), KCNC3 (0, 3, 3), TPK1 (0, 3, 3),
PROM1 (0, 3, 3), PKD1L2 (0, 3, 3),
JMJD1C (1, 2, 5), CR1 (1, 1, 3), WISP3 (1, 2, 3), ZBTB11 (1, 2, 3), NUP210L (1, 1, 3),
FAM35A (1, 1, 2), FLJ23867 (1, 1, 2), KCNK13 (1, 1, 2), SORL1 (0, 2, 5), CCDC6 (0, 2, 2),
PTGS1 (0, 2, 8), ANXA11 (0, 1, 6), RBP1 (0, 1, 6), CASP2 (0, 2, 5), SYT1 (0, 2, 5), RNF44 (0, 2, 4),
LETMD1 (0, 2, 4), RDH13 (0, 2, 4), TRIM28 (0, 1, 4), LST1 (0, 0, 4), SUPV3L1 (0, 2, 4), TARP (0, 0, 4),
GNG4 (0, 2, 4), TCERG1 (0, 1, 4), RAD23A (0, 1, 4), PPP1R12C (0, 2, 4), SRRT (0, 2, 4), MRI1 (0, 2, 4),
CUX1 (0, 1, 4), RAB8A (0, 1, 4), FAM32A (0, 1, 4),
BRD7 (0, 2, 2), CAPN13 (0, 2, 2), CHTOP (0, 2, 3), DUSIL (0, 2, 2), EMR2 (0, 2, 3),
EPOR (0, 2, 3), GATAD2A (0, 2, 2), GTF2F1 (0, 2, 3), IGSF23 (0, 2, 2), KHSRP (0, 2, 2),
LSM4 (0, 2, 3), MACROD2 (0, 2, 2), MED23 (0, 2, 3), MLL2/KMT2D (0, 2, 4), NCOA4 (0, 2, 3), ORM1 (0, 2, 2), PET100 (0, 2, 2), PKN1 (0, 2, 2), POLD1 (0, 2, 2), PTCD1 (0, 2, 2), RDH10 (0, 2, 2), RFXANK (0, 2, 2), RHBDD1 (0, 2, 3), SETDB1 (0, 2, 3), SMARCA4 (0, 2, 3), SULTIE1 (0, 2, 2), TNPO2 (0, 2, 2), UPF1 (0, 2, 2), VTCN1 (0, 2, 3), ZNF212 (0, 2, 2), ZNF248 (0, 2, 2), ZNF282 (0, 2, 3), ZNF444 (0, 2, 2), ZNF787 (0, 2, 2),
CDSN (0, 1, 3), DIAPH1 (0, 1, 3), FBRSL1 (0, 1, 3), FIZ1 (0, 1, 3), FRY (0, 1, 3), GPR132 (0, 1, 3), GRK6 (0, 1, 3), NAA38 (0, 1, 3), NDUFAF4 (0, 1, 3), PGS1 (0, 1, 3), PPP6R1 (0, 1, 3), SCGB2A1 (0, 1, 3), TACR1 (0, 1, 3), TIMM44 (0, 1, 3), TMEM254 (0, 1, 3), VRK3 (0, 1, 3), ZNF579 (0, 1, 3), ZNF581 (0, 1, 3),
BCLAF1 (0, 0, 3), CAPRIN1 (0, 0, 3), DDX39A (0, 0, 3), EDNRA (0, 0, 3), GPT2 (0, 0, 3), IGFL1 (0, 0, 3),
KLF8 (0, 0, 3), MUC7 (0, 0, 3), PRRC2C (0, 0, 3), SHMT2 (0, 0, 3), TNNC1 (0, 0, 3),
AGAP7 (0, 1, 2), ARF3 (0, 1, 2), ARHGEF5 (0, 1, 2), ARRDC2 (0, 1, 2), ASPSCR1 (0, 1, 2), ATMIN (0, 1, 2), BANK1 (0, 1, 2), BRWD1 (0, 1, 2), CBX5 (0, 1, 2), CCNT1 (0, 1, 2), CDK3 (0, 1, 2), CELA1 (0, 1, 2),
CNOT4 (0, 1, 2), COA3 (0, 1, 2), CREBZF (0, 1, 2),
CST7 (0, 1, 2), DSC3 (0, 1, 2), DUS3L (0, 1, 2), EPS15L1 (0, 1, 2), FAM71E1 (0, 1, 2), FCHO1 (0, 1, 2),
FKBP4 (0, 1, 2), FNBP4 (0, 1, 2), GCSH (0, 1, 2), ICOSLG (0, 1, 2), ILF3 (0, 1, 2), JUND (0, 1, 2), KXD1 (0, 1, 2), LARS (0, 1, 2), MKX (0, 1, 2), MPHOSPH6 (0, 1, 2), NMNAT3 (0, 1, 2), NOP14 (0, 1, 2), RAI2 (0, 1, 2), RANBP3 (0, 1, 2), RGMA (0, 1, 2), RGS19 (0, 1, 2), RTKN2 (0, 1, 2), SCNN1G (0, 1, 2), SDCCAG3 (0, 1, 2), SIN3B (0, 1, 2), SIRT7 (0, 1, 2), SLC25A36 (0, 1, 2), TAF1C (0, 1, 2), TMCC1 (0, 1, 2), TRMT1 (0, 1, 2), YAF2 (0, 1, 2), ZBTB39 (0, 1, 2), ZBTB44 (0, 1, 2), ZNF644 (0, 1, 2),
ACAT2 (0, 0, 2), ADIRF (0, 0, 2), ALKBH7 (0, 0, 2), ANKRD10-IT1 (0, 0, 2), AP3M1 (0, 0, 2), ARAP1 (0, 0, 2), ARHGAP25 (0, 0, 2), ARHGEF7 (0, 0, 2), ARID2 (0, 0, 2), ATF5 (0, 0, 2), ATG4D (0, 0, 2), CD48 (0, 0, 2), CYP2E1 (0, 0, 2), DDA1 (0, 0, 2), DHPS (0, 0, 2), DNAJC2 (0, 0, 2), EBP (0, 0, 2), EHMT1 (0, 0, 2),
EMX2OS (0, 0, 2), ING4 (0, 0, 2), INPP5D (0, 0, 2), INSL3 (0, 0, 2), KANSL2 (0, 0, 2), KAT6B (0, 0, 2),
KMO (0, 0, 2), LYRM1 (0, 0, 2), OGT (0, 0, 2), OR12D3 (0, 0, 2), OTUD5 (0, 0, 2), PLA2G7 (0, 0, 2), PLEK (0, 0, 2), PROK2 (0, 0, 2), SAR1A (0, 0, 2), SLC38A2 (0, 0, 2), SNAP23 (0, 0, 2), SNRPE (0, 0, 2), SP1 (0, 0, 2), TRPC2 (0, 0, 2), VAV1 (0, 0, 2), WAC (0, 0, 2), WAPAL (0, 0, 2),
FCRL2 (1, 1, 1), IL23A (1, 1, 1), KCNA6 (1, 1, 1), MAP2K7 (1, 1, 1), MRPL41 (1, 1, 1), NACC1 (1, 1, 1),
PDCL2 (1, 1, 1), PPP1R3F (1, 1, 1), RASL10B (1, 1, 1), RCVRN (1, 1, 1), ZNF224 (1, 1, 1),
KARS (0, 0, 1), AMPD3 (0, 0, 1), EIF4B (0, 0, 1), NACA (0, 0, 1), PMS2P1 (0, 0, 1), SMARCC2 (0, 1, 1),
ZDHHC17 (0, 0, 1), CCT2 (0, 0, 1), COPZ1, EIF3C, OGFOD2,
ATP5G2, EIF2D, GABARAPL2, LARP4, TRAFD1, HIST1H3D, ISCU, PMS2P5, THYN1, TRIAP1, ZNF616, ATP5L, ANK3, CBFA2T3, ASB3, AAAS, ATP5O.

### “GENE Y” Genes

whose lesser mRNA expression in cancer is observed to correlate with greater cancer susceptibility to anti-cancer activity of almitrine dimesylate:
ARL6IP5 (0, 2, 7), CRAT (0, 2, 4), CD58 (0, 0, 6), ITGB5 (0, 0, 6), VCP (0, 0, 6), RAPH1 (0, 0, 5),
PTPLAD1 (0, 0, 5), SMAD5 (0, 0, 4), VAMP3 (0, 0, 4), LMNA (0, 0, 4), EMP2 (0, 0, 4), RAB23 (0, 0, 1),
ABHD12 (0, 0, 3), ALDH9A1 (0, 0, 3), GLT8D1 (0, 0, 3), MAP4 (0, 0, 3), MPRIP (0, 0, 3), RER1 (0, 0, 3),
SAMD4A (0, 0, 3), SCARB2 (0, 0, 3), SEC22C (0, 0, 3), SEC23A (0, 0, 3), TCEB3 (0, 0, 3), TINF2 (0, 0, 3),
TM9SF1 (0, 0, 3), TMBIM1 (0, 0, 3),
TMEM107 (0, 0, 3), TMEM59 (0, 0, 3),
NBR1 (0, 1, 2), NCKAP1 (0, 1, 2), SORT1 (0, 1, 2),
AFF4 (0, 0, 2), ANKH (0, 0, 2), ATP11B (0, 0, 2), ATP13A2 (0, 0, 2), B3GALTL (0, 0, 2), BBIP1 (0, 0, 2),
FLJ42351 (0, 0, 2), CHMP5 (0, 0, 2), CLCC1 (0, 0, 2), CYTH3 (0, 0, 2), DYNLL1 (0, 0, 2), EMC1 (0, 0, 2),
EMC7 (0, 0, 2), EXOC5 (0, 0, 2), FAHD1 (0, 0, 2),
GAB1 (0, 0, 2), GNA11 (0, 0, 2), GNE (0, 0, 2), GRHPR (0, 0, 2), HEXA (0, 0, 2), LPIN1 (0, 0, 2), MAGI3 (0, 0, 2), MED27 (0, 0, 2), MELK (0, 0, 2), MFN1 (0, 0, 2), MIOS (0, 0, 2), MPDZ (0, 0, 2), MPG (0, 0, 2),
MUT (0, 0, 2), MYO5B (0, 0, 2), NAGLU (0, 0, 2),
NDUFA4 (0, 0, 2), NGRN (0, 0, 2), NOL6 (0, 0, 2), NRM (0, 0, 2), PDIA6 (0, 0, 2), PRC1 (0, 0, 2), RAC1 (0, 0, 2), RAPGEF1 (0, 0, 2), RGL2 (0, 0, 2), RIN3 (0, 0, 2), RRBP1 (0, 0, 2), RRM2 (0, 0, 2), SEC11A (0, 0, 2),
SFMBT1 (0, 0, 2), SLC25A1 (0, 0, 2), SLC25A24 (0, 0, 2), SNX13 (0, 0, 2), SPATA5 (0, 0, 2), SURF4 (0, 0, 2), TMEM179B (0, 0, 2), TMEM8B (0, 0, 2), TMF1 (0, 0, 2), TUBB3 (0, 0, 2), TUBG2 (0, 0, 2), UBA3 (0, 0, 2), UBN2 (0, 0, 2), UCK1 (0, 0, 2), ZDHHC3 (0, 0, 2), ZWILCH (0, 0, 2),
ODF2L (0, 0, 1), HOOK3 (0, 0, 1), BMPR2 (0, 0, 1), DZIP1 (0, 0, 1), CBR1 (0, 0, 1), APMAP (0, 0, 1),
DNAJB5 (0, 0, 1), CAMK2D, DACT1, MAP3K6, RALB, COL1A2, DDR2, COPS8, BNC2, ACTC1, EBF1,
DAB2IP, CLOCK, CRK, ARPC2, COL5A3, CDK20, CTTNBP2NL, ACVR2A, DIP2C, CCDC93, CAMTA2, AKIRIN2.

### Method Variant

In the gene lists below, generated from NCI COMPARE data, the cancer gene expression correlations are ranked by summing the |R| values, greater than 0.3, for each gene expression correlation to almitrine dimesylate susceptibility. This method variant simultaneously captures, in a single parameter (Σ|R|, where |R|>x, wherein x is 0.3 but can be higher or lower in other embodiments), the dual importance of amplitude, and frequency of observation, of a gene expression correlation. Preferably, for a gene, if/when all its correlations greater than x are not of the same sign, then this gene is dismissed from incorporation in this prediction model, for this drug. This method, at least with the 0.3 cutoff, more highly ranks some genes than the prior method, e.g. PTGS1, RBP1, JMJD1C, SORL1, ANXA11, CASP2, SYT1. In alternative disclosure embodiments this method, and/or component(s)/variant(s)/derivative(s) thereof, is applied with a different, optionally candidate (or failed candidate), optionally FDA/EMA/MHRA/PMDA/NMPA licensed, cancer drug(s), optionally one or more of the named/specified cancer drugs herein.

### “GENE Q” Genes

SCAF11 (4.838), SLC38A1 (4.295), TMPRSS3 (3.612), DNAJC12 (3.588), PGLS (3.29), PTGS1 (3.155), USP36 (3.13), ZCCHC6 (2.861), MYC (2.839), CBLL1 (2.684), PCID2 (2.477), RNF144B (2.439), TRIM13 (2.414), RBP1 (2.363), JMJD1C (2.202), CAMKK2 (2.115), MAU2 (2.07), CASP2 (2.063), SLC35E1 (2.039), DDX49 (2.017), SORL1 (2.006), SYT1 (1.96), ANXA11 (1.958), ZNF766 (1.87), RNF44 (1.73), ILF2 (1.713), DDX41 (1.709), NADSYN1 (1.69), HMX1 (1.685), PPP1R12C (1.663), MROH1 (1.652), NPTX2 (1.652), LETMD1 (1.642), MRI1 (1.635), SUPV3L1 (1.631), HNRNPA1 (1.628), NRBF2 (1.624), RDH13 (1.623), ZNF277 (1.622), TRIM28 (1.617), SRRT (1.584), RAD23A (1.582), GNG4 (1.58), FAM32A (1.562), CUX1 (1.545), TCERG1 (1.538), RAB8A (1.532), TARP (1.506), LST1 (1.499), WISP3 (1.439), TPK1 (1.433), FABP6 (1.426), PROM1 (1.353), NUP210L (1.353), KCNC3 (1.339), ZBTB11 (1.327), PTH2R (1.315), CR1 (1.279), PKD1L2 (1.233), FLJ23867 (0.936), KCNK13 (0.9), FAM35A (0.899), CCDC6 (0.863).

### “GENE Y” Genes

ARL6IP5 (2.563), VCP (2.093), CD58 (2.013), ITGB5 (1.922), PTPLAD1 (1.682), RAPH1 (1.666), CRAT (1.497), EMP2 (1.346), VAMP3 (1.324), SMAD5 (1.305), LMNA (1.254).

### Alternative Method Variant

Ranking gene expression correlations to susceptibility/resistance to a cancer drug(s) by:

Σ|R| for a gene (defined prior), divided by the number of probes for that same gene in the gene expression database(s) used: Σ|R|/probe number. This method controls for the fact that different genes are likely to have a different probe number in the gene expression database(s). So, it yields a more equitable ranking, with the relative ordering of gene expression correlations likely to be more accurately represented. To illustrate by example: cancer gene expression correlations are ranked by summing the |R| values, greater than 0.3 (in other embodiments another value is selected, e.g. greater—closer to 1—for greater stringency), for each gene expression correlation to almitrine dimesylate susceptibility and dividing each Σ|R| by the number of probes for that gene in the gene expression database(s) used (data not shown). In alternative disclosure embodiments this method, and/or component(s)/variant(s)/derivative(s) thereof, is applied with a different, optionally candidate (or failed candidate), optionally FDA/EMA/MHRA/PMDA/NMPA licensed, cancer drug(s), optionally one or more of the named/specified cancer drugs herein.

### Alternative Method Variant

In the gene lists below, generated from NCI COMPARE data, the cancer gene expression correlations are ranked by summing the |R| values, greater than 0.3 (in other embodiments another value is selected, e.g. greater—closer to 1—for greater stringency), for each gene expression correlation to almitrine dimesylate susceptibility but wherein only one transcript sequence, per gene, per gene expression database is used in the calculation of Σ|R|, wherein the transcript sequence with the highest value of |R| is selected for each gene per database. This stipulation controls for the fact that different genes can have different numbers of transcript sequence variants, each probed for, in any given gene expression database. Even with this control, there is still margin for bias if one or more genes are missing from one or more of the gene expression databases utilized, optionally which can be controlled for by only utilizing gene expression databases that contain data for all the same genes (data not shown). However, although these biases can impact the relative ordering of the gene expression correlations to drug(s) susceptibility/resistance this is not so important. I would argue. More useful is when all the different transcript sequences for each gene are considered, and are not controlled away, then the most highly ranked gene expressions, assuming a reasonably stringent cut-off value of R| is selected, are likely to be extremely valid and reliable. This is valuable. So, the method variant introduced by this section is not actually the most favoured for many use cases. In alternative disclosure embodiments this method, and/or component(s)/variant(s)/derivative(s) thereof, is applied with a different, optionally candidate (or failed candidate), optionally FDA/EMA/MHRA/PMDA/NMPA licensed, cancer drug(s), optionally one or more of the named/specified cancer drugs herein.

### “GENE Q” Genes

RBP1 (2.363), MAU2 (2.07), DNAJC12 (2.044), DDX49 (2.017), MYC (1.997), ANXA11 (1.958), ZNF766 (1.87), CBLL1 (1.859), SLC38A1 (1.83), JMJD1C (1.81), SCAF11 (1.73), RNF44 (1.73), TRIM13 (1.681), DDX41 (1.709), NADSYN1 (1.69), ZCCHC6 (1.674), CASP2 (1.672), PGLS (1.665), NPTX2 (1.652), SUPV3L1 (1.631), HNRNPA1 (1.628), TRIM28 (1.617), PTGS1 (1.594), SORL1 (1.574), FAM32A (1.562), RAB8A (1.532), WISP3 (1.439), FABP6 (1.426), PROM1 (1.353), NUP210L (1.353), TMPRSS3 (1.345), KCNC3 (1.339), USP36 (1.332), ZBTB11 (1.327), ILF2 (1.323), PTH2R (1.315), CAMKK2 (1.283), MROH1 (1.282), HMX1 (1.274), PPP1R12C (1.264), ZNF277 (1.25), LETMD1 (1.248), PKD1L2 (1.233), NRBF2 (1.217), RAD23A (1.211), RDH13 (1.209), PCID2 (1.199), SYT1 (1.195), SRRT (1.171), CUX1 (1.168), TCERG1 (1.159), TPK1 (0.96), SLC35E1 (0.888), MRI1 (0.838), RNF144B (0.835), GNG4 (0.796), LST1 (0.766), CR1 (0.74), TARP (0.383).

### “GENE Y” Genes

ITGB5 (1.62), ARL6IP5 (1.522), VCP (1.406), EMP2 (1.346), SMAD5 (1.305), LMNA (1.254), CRAT (1.095), TMEM59 (1.026), SAMD4A (1.009), RAPH1 (1.007), MPRIP (1.004), TMBIM1 (0.959), TINF2 (0.95), ALDH9A1 (0.972), TCEB3 (0.944), SCARB2 (0.723), VAMP3 (0.701), CD58 (0.693), SEC22C (0.689), TM9SF1 (0.683), PTPLAD1 (0.668), SEC23A (0.662), ABHD12 (0.658), RER1 (0.647), MAP4 (0.646), GLT8D1 (0.622), TMEM107 (0.613), RAB23 (0.334).

### RNA-Seq Data

Gene entries in the table below: their gene expression correlates (consistently positively or negatively) with the anti-cancer activity of almitrine dimesylate. Both when their gene expression is assayed by a microarray or RNA-seq method. Indeed, for all those shown in this table, with both Affymetrix microarray mean and RNA-seq data (either composite and/or an individual isoform[s]) from CellMiner: |R|>0.3. Moreover, |R|>0.3, with microarray data from the NCI COMPARE database, multiple times, and always of same sign. Note that each Affymetrix microarray related value from CellMiner is a mean of 4 different Affymetrix microarray data sets. So, given all this, the gene expression correlations presented in this table are extremely robust.

To conserve space in the following table, only the gene isoforms with correlation |R|>0.3 are shown. Where only one gene isoform exists in the CellMiner database, “Only one” is written in the isoform column. In the table below: (A) Number of correlation observations to almitrine dimesylate anti-cancer activity, at |R|>0.3, in six selected (the most reliable) NCI COMPARE microarray data sets. (B) This is a daughter of column (A): sum of correlation (|R|) values at |R|>0.3. (C) Correlation (R) between mean gene expression (log 2[intensity], using the one microarray data set in CellMiner sourced using a microarray chip made by Agilent) and the mean[1-5]dose(10 μM) parameter (defined prior herein). (D) Correlation (R) between mean gene expression (log 2[intensity], mean of the 4 different microarray data sets in CellMiner produced using microarray chips made by Affymetrix) and mean[1-5]dose(10 μM). (E) Correlation (R) between RNA-seq composite gene expression (log 2[FPKM+1]) and mean gene expression (log 2[intensity], mean of the 4 different Affymetrix microarray data sets), all from CellMiner. (F) Correlation (R) between RNA-seq composite gene expression (log 2[FPKM+1]) and mean[1-|5-]dose(10 μM). (G) Correlation (R) between RNA-seq isoform(s) (log 2[FPKM+1]) and mean[1-/5-]dose(10 μM).

| GENE | A | B | C | D | E | F | G | | | |
|---|---|---|---|---|---|---|---|---|---|---|
| SCAF11 | 11 | 4.838 | 0.4955 | 0.5109 | 0.63 | 0.4901 | Only one | | | |
| | | | | | | | NM_021800 | NM_201262 | | |
| DNAJC12 | 9 | 3.588 | Absent | 0.487 | 0.877 | 0.5178 | 0.5107 | 0.4978 | | |
| | | | | | | | NM_032404 | NM_032405 | | |
| TMPRSS3 | 9 | 3.612 | 0.333 | 0.4664 | 0.717 | 0.396 | 0.4514 | 0.3152 | | |
| | | | | | | | NM_000962 | NM_001271165 | NM_001271368 | NM_080591 |
| PTGS1 | 8 | 3.155 | 0.3431 | 0.4235 | 0.952 | 0.4104 | 0.331 | 0.3403 | 0.343 | 0.3995 |
| MYC | 8 | 2.839 | 0.3052 | 0.3455 | 0.82 | 0.3072 | Only one | | | |
| ITGB5 | 6 | 1.922 | −0.3143 | −0.3035 | 0.898 | −0.3314 | Only one | | | |
| | | | | | | | NM_203365 | NM_213589 | | |
| RAPH1 | 5 | 1.666 | −0.4392 | −0.4781 | 0.861 | −0.4045 | −0.3007 | −0.4316 | | |
| | | | | | | | NM_024814 | | | |
| CBLL1 | 6 | 2.684 | 0.349 | 0.409 | 0.662 | 0.3772 | 0.3731 | | | |
| | | | | | | | NM_032983 | | | |
| CASP2 | 5 | 2.063 | 0.3845 | 0.4075 | 0.554 | 0.3784 | 0.4086 | | | |
| SORL1 | 5 | 2.006 | 0.4329 | 0.396 | 0.941 | 0.3576 | Only one | | | |
| | | | | | | | NM_005639 | | | |
| SYT1 | 5 | 1.96 | 0.1757 | 0.3083 | 0.934 | 0.3683 | 0.3615 | | | |
| | | | | | | | NM_003171 | | | |
| SUPV3L1 | 4 | 1.631 | 0.3038 | 0.3229 | 0.48 | 0.3407 | 0.3432 | | | |
| ZNF766 | 4 | 1.87 | 0.3334 | 0.4441 | 0.573 | 0.4663 | Only one | | | |
| ZNF277 | 4 | 1.622 | 0.5117 | 0.4562 | 0.496 | 0.4765 | Only one | | | |
| | | | | | | | NM_004515 | | | |
| ILF2 | 4 | 1.713 | 0.4322 | 0.4045 | 0.49 | 0.3507 | 0.3485 | | | |
| | | | | | | | NM_018942 | | | |
| HMX1 | 4 | 1.685 | Absent | 0.3306 | 0.217 | 0.3326 | 0.3711 | | | |
| | | | | | | | NM_001040442 | | | |
| FABP6 | 3 | 1.426 | Absent | 0.3054 | 0.897 | 0.3563 | 0.4287 | | | |
| | | | | | | | NM_004977 | NR_110912 | | |
| KCNC3 | 3 | 1.339 | Absent | 0.3701 | 0.897 | 0.3426 | 0.3726 | 0.3267 | | |
| | | | | | | | NM_022445 | | | |
| TPK1 | 3 | 1.433 | 0.4196 | 0.408 | 0.758 | 0.4117 | 0.4072 | | | |
| | | | | | | | NM_172215 | | | |
| CAMKK2 | 5 | 2.115 | 0.4571 | 0.4525 | 0.641 | 0.226 | 0.3236 | | | |
| | | | | | | | NR_033677 | | | |
| DDX49 | 5 | 2.017 | 0.3517 | 0.327 | 0.466 | 0.0767 | 0.3016 | | | |
| | | | | | | | NM_015416 | | | |
| LETMD1 | 4 | 1.642 | 0.4387 | 0.4206 | 0.613 | 0.2881 | 0.3956 | | | |
| | | | | | | | NM_016277 | | | |
| RAB23 | 1 | 0.334 | −0.437 | −0.4617 | 0.57 | −0.2545 | −0.3728 | | | |

Refer to FIG. 1, which graphs some selected cancer gene expression correlations to almitrine dimesylate susceptibility, drawn from the favoured table above. These graphed correlations have a prominent number of observations, at sizable correlation value, in NCI COMPARE and a sizable correlation value with both CellMiner microarray (Affymetrix and Agilent chips) and RNA-seq data. This selection criteria is favoured for selecting which gene(s) to incorporate into a prediction model for predicting which cancer(s) are susceptible/resistant to almitrine dimesylate anti-cancer activity. In other embodiments, this method, and/or component(s)/variant(s)/derivative(s)/equivalent(s) thereof, can be applied to other anti-cancer drug(s), optionally approved by the FDA/EMA/MHRA/PMDA/NMPA, optionally for anti-cancer use.

In the table above there are a few cases where the composite RNA-seq value doesn't correlate as well as an individual isoform(s). An especially good example of this is with DDX49, wherein the composite correlation is very poor, but the correlation of a single isoform (NR_033677) is good. In some disclosure embodiments, a cancer patient(s)/subject(s) is selected for almitrine (and/or a pharmaceutically-acceptable salt, solvate, hydrate or prodrug thereof) administration on the basis of their level of expression of one or more isoforms (considered independently and/or in combination e.g. combined by some mathematical operation(s); optionally one or more isoforms mentioned herein) in their normal and/or cancer cells. There are some cases not shown in the table because, whilst their correlation to cancer susceptibility/resistance to almitrine dimesylate with microarray data is good, that with RNA-seq data, composite and individual isoform(s), is poor e.g. CD58, SMAD5 (shown below, with all isoforms present in CellMiner for these genes shown). If/when cancer susceptibility to almitrine dimesylate is to be assayed using RNA-seq technology it is better to not employ such gee(s).

| GENE | A | B | C | D | E | F | G | | |
|---|---|---|---|---|---|---|---|---|---|
| | | | | | | | NM_001144822 | NM_001779 | NR_026665 |
| CD58 | 6 | 2.013 | Absent | −0.3832 | 0.62 | −0.0243 | −0.1306 | −0.0044 | 0.0562 |
| | | | | | | | NM_001001419 | NM_001001420 | NM_005903 |
| SMAD5 | 4 | 1.305 | −0.4196 | −0.3461 | 0.474 | −0.0767 | 0.0037 | −0.1328 | −0.0118 |

### Interim Summary

In some disclosure embodiments, a cancer patient(s)/subject(s) is selected for almitrine (and/or a pharmaceutically-acceptable salt, solvate, hydrate or prodrug thereof) administration on the basis of the level of expression of one or more genes (considered independently and/or in combination, e.g. combined by some mathematical operation(s); preferably one or more genes mentioned herein) in their cancer (and/or normal) cells.

Optionally SCAF11, wherein higher SCAF11 gene expression in the subject's cancer, and/or wherein the greater the product of SCAF11 gene expression minus RAPH1 gene expression in a subject's cancer, is a (or the) drive to a decision to administer almitrine (and/or a pharmaceutically-acceptable salt, solvate, hydrate or prodrug thereof) to the subject. A method of, in the cancer (and/or a sample thereof) of a subject, measuring (optionally ex vivo/in vitro) the gene expression of a gene(s) that (as reported herein) positively correlates with the anti-cancer activity of almitrine dimesylate, and/or a gene(s) that (as reported herein) negatively correlates with the anti-cancer activity of almitrine dimesylate, optionally wherein this gene expression data is amalgamated/pooled/synthesized/mathematically operated upon (e.g. as disclosed herein), wherein this data is used to decide whether to administer (preferably a therapeutically effective amount of) almitrine dimesylate to the subject, optionally used to select which subject(s) to enter into a clinical trial of almitrine dimesylate for anti-cancer treatment, is componentry to this disclosure. In a disclosure method embodiment(s), if/when a subject's cancer(s) has high expression of a gene(s) that correlates with cancer susceptibility to almitrine dimesylate (e.g. as reported herein), and/or low expression of a gene(s) that negatively correlates with cancer susceptibility to almitrine dimesylate (e.g. as reported herein), almitrine dimesylate is administered to this subject. In a disclosure method embodiment(s), whether a gene expression in a cancer is high or low is decided by assaying whether it is higher or lower than the gene expression in a different cancer(s), e.g. in a different subject(s), e.g. in a cancer cell line(s), preferably a cancer(s) of same/similar type, and/or the gene expression in the normal tissue of the subject, preferably wherein this normal tissue is that from which the cancer derives, and/or by reference to a reference gene(s) expression in the subject's cancer and/or normal tissue(s). In a disclosure method embodiment(s), whether a gene expression in a cancer is high or low is decided by assaying whether it is higher or lower than the mean/median expression of that gene in a cohort of cancers, preferably of the same cancer type, and/or if the expression of that gene is in a top bracket (non-limiting e.g. half, quartile, decile, 20%, 15%, percentile etc.) of a group of cancers, preferably of same type, from different subjects. In a disclosure method embodiment(s), whether a gene expression in a cancer is high or low is decided by using the RPKM related method of [12].

In a disclosure method embodiment(s), whether a cancer, optionally inside a subject, is susceptible to almitrine (and/or a pharmaceutically-acceptable salt, solvate, hydrate or prodrug thereof) is predicted by comparing whether its gene expression of one or more of “GENE Q” is equal or higher (and/or substantially similar), and/or its expression of one or more of “GENE Y” is equal or lower (and/or substantially similar), than a cancer(s) (optionally a cancer cell line[s]) known to be susceptible to almitrine (and/or a pharmaceutically-acceptable salt, solvate, hydrate or prodrug thereof) treatment, optionally wherein this prediction is incorporated into an oral/audio/written/electronically encoded/computer medium report (optionally accessible on a computer/phone/electronic device, optionally transmitted over the internet/intranet, optionally transmitted by electromagnetic radiation) and/or is used to inform/dictate the decision as to whether to administer almitrine (and/or a pharmaceutically-acceptable salt, solvate, hydrate or prodrug thereof) to the subject that has this cancer, wherein if/when the subject's cancer is predicted to be susceptible to almitrine by this method then the subject is administered with a therapeutically effective amount of almitrine (and/or a pharmaceutically-acceptable salt, solvate, hydrate or prodrug thereof), optionally in co-therapy with another cancer treatment(s), optionally one or more licensed by the FDA and/or EMA. Conversely, in a different disclosure embodiment(s), whether a cancer, optionally inside a subject, is non-responsive/resistant to almitrine (and/or a pharmaceutically-acceptable salt, solvate, hydrate or prodrug thereof) is predicted by comparing whether its gene expression of one or more of “GENE Q” is equal or lower (and/or substantially similar), and/or its expression of one or more of “GENE Y” is equal or higher (and/or substantially similar), than a cancer(s) (optionally a cancer cell line[s]) known to be non-responsive/resistant to almitrine (and/or a pharmaceutically-acceptable salt, solvate, hydrate or prodrug thereof) treatment, optionally wherein this prediction is incorporated into an oral/audio/written/electronically encoded/computer medium report (optionally accessible on a computer/phone/electronic device, optionally transmitted over the internet/intranet, optionally transmitted by electromagnetic radiation) and/or is used to inform/dictate the decision as to whether to administer almitrine (and/or a pharmaceutically-acceptable salt, solvate, hydrate or prodrug thereof) to the subject that has this cancer, wherein if/when the subject's cancer is predicted to be resistant to almitrine by this method then the subject is not administered with almitrine (and/or a pharmaceutically-acceptable salt, solvate, hydrate or prodrug thereof), optionally wherein the subject is administered a different cancer treatment(s) instead.

Componentry to this disclosure is a method to search for a correlation between a drug(s)/compound(s) activit[y/ies], in vitro and/or in vivo, upon a biological sample/tissue/system/organism, optionally anti-cancer activity, and the mean gene expression of more than one gene, which all correlate in the same direction (+ or −) with the drug(s) activity, and/or the product of the gene expression of a gene(s) subtracted from that of another gene(s), wherein the subtracted gene(s) has an opposite correlation, and/or the mean of gene expression of a gene(s) and one or more products of the gene expression of a gene(s) subtracted from that of another gene(s). Non-limiting examples of this method can be found in the present disclosure. With this method in hand, variations on this method will be apparent to those of the art, which are also componentry to the present disclosure. Once such a correlation has been elucidated it can be used as input into the decision whether to administer the drug(s) to a subject(s), e.g. if/when the subject's cancer has a high score for a gene expression function, which a drug(s) efficacy has been shown to scale/correlate with, then this drug(s) is administered to the subject.

In alternative disclosure embodiments, instead of, or in addition to, looking at cancer gene expression(s) by assaying at the transcript level, protein(s) amount is assayed, wherein with this data, the same gene(s) can be used to predict cancer susceptibility to almitrine dimesylate (transcript and protein amounts correlate [13]). A protein(s) amount can be assayed, for non-limiting example, using labelled antibodies to the protein. Especially convenient for a membrane protein, which has a component part(s) outside of the cell, e.g. CD58, CD133 etc.

### Formulae

“GENE Q” is any gene—as disclosed herein—(or mutant/variant thereof) whose greater expression in cancer (at DNA and/or mRNA and/or protein levels) correlates with greater cancer susceptibility to anti-cancer activity of almitrine (and/or a pharmaceutically-acceptable salt, solvate, hydrate or prodrug thereof) (e.g. SCAF11, SLC38A1, MYC etc.), or is the mean gene expression of a number of such genes. “GENE Y” is any gene—as disclosed herein—(or mutant/variant thereof)—whose lesser expression in cancer (at DNA and/or mRNA and/or protein levels) correlates with greater cancer susceptibility to anti-cancer activity of almitrine (and/or a pharmaceutically-acceptable salt, solvate, hydrate or prodrug thereof) (e.g. RAPH1, RAB23 etc.), or is the mean gene expression of a number of such genes.

“GENE Q” and “GENE Y” terms, when used in this disclosure, can in further embodiments, independently at each point of use, refer to additional entities comprising:

In some embodiments, “GENE Q” is the mean of a number (n) of “GENE Q”:

GENE Q=(ΣⁿGENE Q)/n

where GENE Q is (optionally normalized and/or mathematically operated upon e.g. logged, e.g. log 2) gene expression amount.

In some embodiments, “GENE Y” is the mean of a number (n) of “GENE Y”:

GENE Y=(ΣⁿGENE Y)/n

where GENE Y is (optionally normalized and/or mathematically operated upon e.g. logged, e.g. log 2) gene expression amount.

In some embodiments, “GENE Q” is “GENE Q” minus “GENE Y”:

GENE Q=GENE Q−GENE Y

where GENE Q and GENE Y are (optionally normalized and/or mathematically operated upon e.g. logged, e.g. log 2) gene expression amounts (or one or both is a mean thereof).

In some embodiments, “GENE Y” is “GENE Y” minus “GENE Q”:

GENE Y=GENE Y−GENE Q

where GENE Y and GENE Q are (optionally normalized and/or mathematically operated upon e.g. logged, e.g. log 2) gene expression amounts (or one or both is a mean thereof).

An outputted value of “GENE Q” by any formula herein can be inputted as input for “GENE Q” into any formula herein. An outputted value of “GENE Y” by any formula herein can be inputted as input for “GENE Y” into any formula herein. This can generate values of “GENE Q” and/or “GENE Y” with very strong predictive power for cancer susceptibility to anti-cancer activity of almitrine (and/or a pharmaceutically-acceptable salt, solvate, hydrate or prodrug thereof). For some examples refer to FIG. 1 and its legend. To pull one example out:
GENE Q=(d, DNAJC12, TMPRSS3, PTGS1, MYC, CBLL1, CASP2, SORL1, SYT1, SUPV3L1, ZNF766, ZNF277, ILF2, HMX1, FABP6, KCNC3, TPK1, CAMKK2, DDX49, LETMD1)/20, where d=(SCAF11-c), where c=(ITGB5, RAPH1, RAB23)/3, where each gene name represents its (optionally normalized and/or mathematically operated upon e.g. logged, e.g. log 2) gene expression value.

A simpler example (FIG. 1):

GENE Q=SCAF11−RAPH1,

where each gene name represents its (optionally normalized and/or mathematically operated upon e.g. logged, e.g. log 2) gene expression value.

Each gene expression value can be raw or, optionally, normalized with the expression of a reference or “housekeeping gene” (non-limiting e.g. [14]) or mean expression of multiple reference/housekeeping genes, or using some other normalization method of the art (e.g. z-scoring, e.g. RPKM method [12] etc.), and when gene expressions are combined/compared it is optimal to combine/compare like with like, e.g. normalized with normalized, wherein the same normalization method is used.

Given these formulae for “GENE Q” and “GENE Y”, modifications/derivatives (e.g. by changing a mathematical symbol[s]) will be apparent to those of the art.

A contemplated prediction score to predict cancer susceptibility to almitrine dimesylate=mean(gene expression of positively correlated gene[s])−mean(gene expression of negatively correlated gene[s])=mean(gene expression of “GENE Q” gene[s])−mean(gene expression of “GENE Y” gene[s]). Optionally this equation can be used with all, or just some fraction of, the “GENE Q” and “GENE Y” genes disclosed herein. Optionally this prediction score can be normalized to a scale from 0 to 100 by a linear transformation of the prediction score of a set of cancer samples from cancer patients. Optionally wherein this score is then used to select which of these cancer patients to administer almitrine (and/or a pharmaceutically-acceptable salt, solvate, hydrate or prodrug thereof) to: those with a prediction score above a selected cut-off value (where in some non-limiting embodiments the cut-off value is a number/integer selected from between w and 100, where w is a number selected from 50, 60, 70, 80, 90).

Assaying for overexpression of “GENE Q”, and/or underexpression of “GENE Y”, in a subject's cancer can be performed (without limitation) by measuring, ex vivo/in vitro, upon a sample(s) drawn from the subject, wherein the sample is (without limitation) derived from “liquid biopsy”, body wash (e.g. a lung wash sample), bodily fluid(s), tissue(s), normal tissue(s), cancer tissue(s) (e.g. sourced from tumour biopsy, e.g. fine needle/core biopsy), suspected cancer tissue(s), circulating cancer cell(s), cell line(s), circulating DNA fragments (cell-free DNA, cfDNA), fragmented RNA, bone marrow, urine, feces, buccal swab, saliva, plasma, serum or whole blood etc. or an extract or processed sample produced from any thereof. How to determine the amount of a DNA/RNA sequence(s), and/or mRNA transcript(s), and/or protein(s), in a biological sample (e.g. in a cancer sample) is well known to those of the art. For non-limiting example, measuring the amount of a DNA/RNA sequence by quantitative real time polymerase chain reaction (qPCR and/or RT-qPCR) and/or Serial Analysis of Gene Expression (SAGE) and/or RNA-Seq and/or whole-exome sequencing and/or Next generation sequencing and/or RNA sequencing and/or microarray chip technology (e.g. nucleic acid hybridization upon a solid-phase nucleic acid biochip array) and/or a “gene expression profiling” method(s), gene amplification in a cancer discovered by Fluorescent In Situ Hybridization (FISH), amount of a specific protein quantified by an immuno/antibody based technique wherein the antibody specifically binds to that protein type forming a protein-antibody complex, the amount of which is then measured etc. Some exemplary methods are used by the Beat-AML Master trial [12]. Componentry to this disclosure is a microarray chip with nucleotide sequences on it complimentary to the mRNA and/or cDNA of one or more of “GENE Q”, and/or one or more of “GENE Y”, and in a further embodiment(s) the method of using it as a custom chip to be used with a biological sample(s) from the subject to diagnose if cancer in the subject is likely to be susceptible to the anti-cancer activity of almitrine (and/or a pharmaceutically-acceptable salt, solvate, hydrate or prodrug thereof). Optionally wherein the chip is “Two-channel”, permitting two samples from the subject to be compared, e.g. one sample from normal, and other from cancer, tissue of subject.

Herein, when “GENE Q” is said to be overexpressed in a cancer this can refer (without limitation) to one or more of the following situations being true:
(a) Its value is higher than the expression of another gene(s) in the same cancer from the same subject, wherein the differential amplitude gives the magnitude of overexpression, e.g. (non-limiting) greater differential between “GENE Q” and “GENE Y” in a cancer shows greater overexpression of “GENE Q”, and/or greater underexpression of “GENE Y”, which predicts (greater) cancer susceptibility to the anti-cancer activity of almitrine (and/or a pharmaceutically-acceptable salt, solvate, hydrate or prodrug thereof);
(b) Its value is higher than the value (for this specification of “GENE Q”) in normal cells/tissue of the subject, preferably wherein this normal tissue is that from which the subject's cancer derives;
(c) Its value is higher than the value(s) (for this specification of “GENE Q”) in a different cancer(s), e.g. from a different subject(s), preferably a cancer(s) of same/similar type;
(d) Its value is higher than the mean/median (for this specification of “GENE Q”) in a cohort of cancers, preferably of the same cancer type;
(e) Its value is in a top bracket (non-limiting e.g. half, quartile, decile, percentile etc.) of values (for this specification of “GENE Q”) of a group of cancers, preferably of same type from different subjects (non-limiting e.g. the cancer gene expression set generated by the Beat AML clinical trial(s) {[1], ClinicalTrials.gov Identifier: NCT03013998} and/or another clinical/“Master trial(s)” e.g. see [15]);
(f) Its value is higher than a standard value or a set of standard values (for this specification of “GENE Q”), above which is predictive of susceptibility to the anti-cancer activity of almitrine (and/or a pharmaceutically-acceptable salt, solvate, hydrate or prodrug thereof);
(g) Its value is higher than a standard value or a set of standard values (for this specification of “GENE Q”) from a normal cell(s) or tissue(s) from healthy individual(s);
(h) Its value is higher than a standard value or a set of standard values (for this specification of “GENE Q”) from a subject(s) (of same species) with the same cancer;
(i) Its value is higher than the median value (of this specification of “GENE Q”) in this cancer type;
(j) Its value is higher than a standard value or a set of standard values (for this specification of “GENE Q”) measured from the subject, and/or a sample(s) from the subject, taken prior to this subject developing cancer(s);
(k) Its value is higher than a control value, which is the “GENE Q” value (for same specification of “GENE Q”) from a control cohort;
(l) Its value is higher than the level (in all, or the mean, or median) in reference cancer samples, preferably wherein these cancer samples are the same type (e.g. same cancer histotype) as each other and as the subject's cancer.
(m) Its value is equal or higher than the level in a cancer susceptible to anti-cancer activity by almitrine (and/or a pharmaceutically-acceptable salt, solvate, hydrate or prodrug thereof).

By the teaching above, when “GENE Y” is said to be underexpressed in a cancer this can refer (without limitation) to one or more of the above situations inverted i.e. lower value of “GENE Y” in the subject's cancer, in relation to one or more comparator(s) mentioned in (a)-(m), indicates a (greater) susceptibility to the anti-cancer activity of almitrine (and/or a pharmaceutically-acceptable salt, solvate, hydrate or prodrug thereof).

A (non-limiting) method to assay overexpression of a “GENE Q”, and/or underexpression of a “GENE Y”, in a subject's cancer is to compare expression in this cancer to expression in the subject's normal tissue, most preferably the tissue from which the cancer derives, wherein this sampling can optionally be done in vitro/ex vivo, and herein wherever this disclosure refers to measuring the expression of one or more of “GENE Q” and/or one or more of “GENE Y” in a subject's cancer, and/or sample(s) thereof, then in alternative embodiments, expression of one or more of “GENE Q” and/or one or more of “GENE Y” is measured in a subject's normal tissue(s) also and comparison is made to determine over- or under-expression of one or more of “GENE Q” and/or “GENE Y” in the subject's cancer, optionally wherein this cancer and normal tissue sampling is conducted in vitro/ex vivo with samples sourced from the subject and their cancer. There are other methods known to those of the art to determine over-/under-gene expression in a cancer, at the DNA (e.g. gene amplification and/or chromosome rearrangement placing gene under control of more active promoter) and/or transcript and/or protein levels, and their use, the use of one or more, in determining cancer susceptibility to almitrine (and/or a pharmaceutically-acceptable salt, solvate, hydrate or prodrug thereof) is contemplated by, and componentry to, this disclosure.

Also componentry to this disclosure is to monitor “GENE Q” and/or “GENE Y” during almitrine (and/or a pharmaceutically-acceptable salt, solvate, hydrate or prodrug thereof) treatment in a subject with cancer, wherein their decreasing overexpression (“GENE Q” case), and/or lessoning degree of underexpression (“GENE Y” case), in a sample(s) sourced from the subject, and/or subject's cancer, can be used to monitor anti-cancer treatment, or lack thereof (no change in “GENE Q” and or “GENE Y” expression), exerted by almitrine (and/or a pharmaceutically-acceptable salt, solvate, hydrate or prodrug thereof) administration and/or other administered drug(s).

### Description Component in Claim Format

Almitrine (and/or a pharmaceutically-acceptable salt, solvate, hydrate or prodrug thereof) for use in the treatment/amelioration/prevention/combat of a cancer (optionally CML/AML, or a lung cancer e.g. Non-Small Cell Lung Cancer [NSCLC]) overexpressing one or more of “GENE Q”, and/or underexpressing one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable.

Before starting almitrine (and/or a pharmaceutically-acceptable salt, solvate, hydrate or prodrug thereof) treatment, a test(s) can be carried out to make sure that the subject's cancer overexpresses one or more of “GENE Q”, and/or underexpresses one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable. Optionally, if/when one or more of these conditions aren't met, then almitrine (and/or a pharmaceutically-acceptable salt, solvate, hydrate or prodrug thereof) is not administered to the subject.

Almitrine (and/or a pharmaceutically-acceptable salt, solvate, hydrate or prodrug thereof) for use in a method for treating/ameliorating/preventing/combating cancer (optionally CML/AML, or a lung cancer e.g. NSCLC) in a subject, wherein the cancer is associated with amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, the method comprising administering (preferably a therapeutically effective amount of) almitrine (and/or a pharmaceutically-acceptable salt, solvate, hydrate or prodrug thereof) to the subject.

Almitrine (and/or a pharmaceutically-acceptable salt, solvate, hydrate or prodrug thereof) for use in a method for treating/ameliorating/preventing/combating cancer (optionally CML/AML, or a lung cancer e.g. NSCLC) in a subject, wherein the subject's cancer has amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, and wherein the method comprises identifying that the subject's cancer has amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable (optionally in vitro/ex vivo e.g. using a sample/derivative of the subject's cancer), and administering (preferably a therapeutically effective amount of) almitrine (and/or a pharmaceutically-acceptable salt, solvate, hydrate or prodrug thereof) to the subject.

Almitrine (and/or a pharmaceutically-acceptable salt, solvate, hydrate or prodrug thereof) for use in a method of treating a subject with cancer (optionally CML/AML, or a lung cancer e.g. NSCLC), wherein the method comprises:
(i) determining whether a test sample from the subject's cancer comprises amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable; and
(ii) if the test sample from the subject's cancer comprises an amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, administering to the subject a therapeutically effective amount of almitrine (and/or a pharmaceutically-acceptable salt, solvate, hydrate or prodrug thereof).

A method for diagnosing and treating almitrine sensitive cancer in a subject comprising: analysing a subject sample for the amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, wherein the subject is diagnosed with almitrine sensitive cancer if/when amplification and/or overexpression of one or more of “GENE Q”, and/or underexpression of one or more of “GENE Y”, is detected and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable; and
administering (preferably a therapeutically effective amount of) almitrine (and/or a pharmaceutically-acceptable salt, solvate, hydrate or prodrug thereof) to the diagnosed subject.

A method for treating cancer (optionally CML/AML, or a lung cancer e.g. NSCLC) in a subject comprising: requesting a test providing the results of an analysis to determine whether the subject's cancer has amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, and if it does, administering (preferably a therapeutically effective amount of) almitrine (and/or a pharmaceutically-acceptable salt, solvate, hydrate or prodrug thereof) to the subject.

A method of diagnosing and treating cancer (optionally CML/AML or a lung cancer e.g. NSCLC) in a subject, said method comprising:
a. obtaining (or starting with) a cancer sample from the subject;
b. detecting whether subject's cancer has amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable;
c. diagnosing the subject, optionally via an oral/written/drawn report/analysis/verdict/graph/picture, with a cancer susceptible to almitrine treatment when the subject's cancer has amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable; and
d. administering a therapeutically effective amount of almitrine (and/or a pharmaceutically-acceptable salt, solvate, hydrate or prodrug thereof) to the diagnosed subject.

A method for diagnosing almitrine sensitive cancer in a subject, wherein the cancer is characterized by the amplification and/or overexpression of one or more of “GENE Q”, and/or underexpression of one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable comprising:
i) obtaining (or starting with) a biological sample from the subject, which contains their cancer or part thereof;
ii) applying a monoclonal antibody specific for a protein product(s) of “GENE Q” or “GENE Y” biomarker to the sample,
wherein presence of the biomarker creates an antibody-biomarker complex;
iii) applying a detection agent that detects the antibody-biomarker complex;
iv) diagnosing almitrine sensitive cancer where the detection agent of step iii) is detected at a magnitude that shows that “GENE Q” is overexpressed, and/or that “GENE Y” is underexpressed, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, in the subject's cancer; and
v) if the subject's cancer is diagnosed as being, or likely to be, almitrine sensitive by prior steps, then almitrine (and/or a pharmaceutically-acceptable salt, solvate, hydrate or prodrug thereof) is administered (preferably a therapeutically effective amount) to the subject.

Method of selecting a subject(s) from a group of subjects with cancer (optionally CML/AML, or a lung cancer e.g. NSCLC) to administer almitrine (and/or a pharmaceutically-acceptable salt, solvate, hydrate or prodrug thereof) to, wherein a subject(s) is selected (optionally after being pre-selected on the basis of one or more other criteria) on the basis that their cancer, compared to other cancer(s) in the group, overexpresses one or more of “GENE Q”, and/or underexpresses one or more of “GENE Y”, and/or wherein the expression differential between at least one “GENE Q” (if/when more than one “GENE Q”, their mean is taken and used) minus at least one “GENE Y” (if/when more than one “GENE Y”, their mean is taken and used) is positive and preferably sizable, optimally wherein a selected subject(s) has greater positive differential in their cancer than those not selected. This can be a method of selecting a subject(s) for a clinical trial of almitrine anti-cancer activity.

A kit comprising almitrine (and/or a pharmaceutically-acceptable salt, solvate, hydrate or prodrug thereof) with material(s) to measure the expression (mRNA and/or protein) of one or more of “GENE Q”, and/or one or more of “GENE Y”, in the subject's cancer and/or in a sample from the subject's cancer (e.g. from a blood draw for a leukemia such as CML/AML), preferably with instructions for use, wherein the kit can be a companion diagnostic (requiring certain result for administration of almitrine (and/or a pharmaceutically-acceptable salt, solvate, hydrate or prodrug thereof) to subject) or a complimentary diagnostic (to inform decision making, but almitrine {and/or a pharmaceutically-acceptable salt, solvate, hydrate or prodrug thereof} can be administered regardless of the test result[s]).

The methodology described herein for almitrine (and/or a pharmaceutically-acceptable salt, solvate, hydrate or prodrug thereof) can be applied for a different drug(s), as contemplated by, and componentry to, this disclosure: using a sample derived from a subject and/or their cancer, to predict the cancer susceptibility/responsiveness/resistance to this drug(s), optionally a FDA/EMA/MHRA/PMDA/NMPA approved cancer drug(s), optionally a drug combination, wherein in a further embodiment(s) this is used to select which drug(s) to administer to the subject, which is then administered (preferably a therapeutically effective amount) to the subject.

### Description Component in More Formal Claim Format

Not all the following claims (or parts thereof) need be implemented: in alternative embodiments one or more claims, and/or part(s) thereof, are excluded, e.g. the normalization step(s) can be excluded so that the method is implemented with raw data. Herein, wherever “GENE Q” is referred to in the singular, in an alternative embodiment(s) it is referred to in the plural, and/or a claim can be iterated for different “GENE Q”, in series, or alternatively executed once for multiple “GENE Q” in parallel. Herein, wherever “GENE Y” is referred to in the singular, in an alternative embodiment(s) it is referred to in the plural, and/or a claim can be iterated for different “GENE Y”, in series, or alternatively executed once for multiple “GENE Y” in parallel.

The claims below also apply to “GENE Y” substituted in place of “GENE Q” but with a difference: less (rather than more) “GENE Y” protein(s) and/or “GENE Y” nucleic acid(s) and/or “GENE Y” gene copy number amount predicts [optionally greater] susceptibility/responsiveness of a subject's cancer to treatment with almitrine (and/or a pharmaceutically-acceptable salt, solvate, hydrate or prodrug thereof).

In alternative embodiments, wherever almitrine is mentioned in the following claim set, a different drug's name (one of the drugs mentioned/taught herein) is substituted in its place, and the “GENE Q” and/or “GENE Y” mentions in these claims then relate to one or more of those (specified/taught herein) for that other drug:
[1] A method to predict (give an indication of; give a probabilistic indication of) the susceptibility/responsiveness of a subject's cancer to almitrine (and/or a pharmaceutically-acceptable salt, solvate, hydrate or prodrug thereof) administration, comprising the steps of:
i) obtaining (or starting with) a biological sample(s) from the subject;
ii) determining the DNA and/or mRNA and/or cDNA and/or protein expression level of one or more of “GENE Q” and/or “GENE Y” in said sample:
measuring the level of one or more of “GENE Q” and/or “GENE Y” protein(s), and/or one or more of “GENE Q” and/or “GENE Y” nucleic acid(s) (DNA/RNA), in a sample(s) obtained from the subject to obtain a value, or values, representing this level(s); and
iii) comparing said expression level(s) to a reference expression level(s):
comparing the value, or values, of the level(s) from step (ii) with a standard/reference value, or a set of standard/reference values, wherein this comparison predicts (gives an indication of; gives a probabilistic indication of) the subject's cancer's susceptibility/responsiveness to almitrine (and/or a pharmaceutically-acceptable salt, solvate, hydrate or prodrug thereof) administration.
[2] The method of Claim 1 wherein a higher level of one or more of “GENE Q” protein(s) and/or “GENE Q” nucleic acid(s), and/or a lower level of one or more of “GENE Y” protein(s) and/or “GENE Y” nucleic acid(s), in the sample relative to the standard/reference value, or a set of standard/reference values, predicts (gives an indication of; gives a probabilistic indication of) [optionally greater] susceptibility/responsiveness of the subject's cancer to treatment with almitrine (and/or a pharmaceutically-acceptable salt, solvate, hydrate or prodrug thereof).
[3] The method of Claim 1, wherein the determination of a higher level of one or more of “GENE Q” protein(s) and/or “GENE Q” nucleic acid(s), and/or a lower level of one or more of “GENE Y” protein(s) and/or “GENE Y” nucleic acid(s), in said sample obtained from the subject is carried out by comparing the measured “GENE Q” and/or “GENE Y” protein(s) level, and/or “GENE Q” and/or “GENE Y” nucleic acid(s) level, in said sample
i) relative to a standard value, or a set of standard values, of the level of “GENE Q” and/or “GENE Y” protein(s), and/or “GENE Q” and/or “GENE Y” nucleic acid(s), from a biological sample(s) from another subject(s) with the same cancer (e.g. same cancer histotype) as the subject; and/or
ii) relative to a standard value, or a set of standard values, of the level of “GENE Q” and/or “GENE Y” protein(s) and/or nucleic acid(s) from one or more cancer cell lines, optionally one or more cancer cell lines derived from the same tissue type as the subject's cancer; and/or
iii) relative to a standard value, or a set of standard values, of the level of “GENE Q” and/or “GENE Y” protein(s), and/or “GENE Q” and/or “GENE Y” nucleic acid(s), from a sample, or samples, from normal cell(s)/tissue(s); and/or
iv) relative to a standard value, or a set of standard values, of the level of “GENE Q” and/or “GENE Y” protein(s), and/or “GENE Q” and/or “GENE Y” nucleic acid(s), from a sample, or samples, obtained from the same subject before any initiation of almitrine (and/or a pharmaceutically-acceptable salt, solvate, hydrate or prodrug thereof) administration;
preferably wherein each “GENE Q” assayed in the biological sample(s) from the subject is compared to the same “GENE Q” type in the standard value(s);
preferably wherein each “GENE Y” assayed in the biological sample(s) from the subject is compared to the same “GENE Y” type in the standard value(s).
[4] The method of any one or more of Claims 1-3, wherein a subject(s) whose cancer has
(i) a higher “GENE Q” protein(s) and/or “nucleic acid(s) amount, above a threshold amount, and/or
(ii) a lower “GENE Y” protein(s) and/or “nucleic acid(s) amount, below a threshold amount, is predicted (giving an indication of; giving a probabilistic indication of) to be (especially) helped/treated by almitrine (and/or a pharmaceutically-acceptable salt, solvate, hydrate or prodrug thereof) administration for anti-cancer therapy, and/or a subject(s) whose cancer has
(iii) a lower “GENE Q” protein(s) and/or “nucleic acid(s) amount, below a threshold amount, and/or
(iv) a higher “GENE Y” protein(s) and/or “nucleic acid(s) amount, above a threshold amount, is not (especially).
[5] A microarray chip comprising/containing a nucleic acid probe(s) for (i.e. a single-stranded nucleotide sequence(s) that is complimentary for/hybridisable to, preferably with ≥85% sequence identity, to) mRNA and/or cDNA (and/or part(s) thereof, e.g. ≥15 consecutive nucleotides thereof) of one or more of “GENE Q” and/or “GENE Y”.
[6] A method of using the microarray chip of Claim 5 comprising contacting it with a sample(s) from the subject, measuring the hybridization, to predict/diagnose if cancer in the subject is likely to be susceptible/responsive to the anti-cancer activity of almitrine (and/or a pharmaceutically-acceptable salt, solvate, hydrate or prodrug thereof) administration.
[7] The method of any one or more of Claims 1-6, wherein a subject(s) whose cancer has
(i) a higher “GENE Q” protein(s) and/or “GENE Q” nucleic acid(s) amount, above a threshold amount, and/or
(ii) a lower “GENE Y” protein(s) and/or “GENE Y” nucleic acid(s) amount, below a threshold amount, is administered a therapeutically effective amount of almitrine (and/or a pharmaceutically-acceptable salt, solvate, hydrate or prodrug thereof), and/or a subject(s) whose cancer has
(iii) a lower “GENE Q” protein(s) and/or “GENE Q” nucleic acid(s) amount, below a threshold amount, and/or
(iv) a higher “GENE Y” protein(s) and/or “GENE Y” nucleic acid(s) amount, above a threshold amount, is not (optionally wherein they are administered with a different cancer treatment(s)/drug(s) instead).
[8] The method of Claim 1, wherein by ranking the level of one or more “GENE Q” protein(s) and/or “GENE Q” nucleic acid(s), and/or the level of one or more “GENE Y” protein(s) and/or “GENE Y” nucleic acid(s), in biological samples from different subjects with cancer, preferably with cancer of same/similar type and/or with shared feature(s), then one can rank which of these cancers (those with most “GENE Q” mRNA/cDNA/protein, and/or those with least “GENE Y” mRNA/cDNA/protein, and/or those with most differential between the amount of “GENE Q” and “GENE Y” mRNA/cDNA/protein) are most likely to be susceptible/responsive to anti-cancer treatment with almitrine (and/or a pharmaceutically acceptable salt, solvate, hydrate or prodrug thereof).
[9] The method of Claim 8, wherein this ranking is used as input into the decision/determination of which subjects with cancer to enter into a clinical trial of almitrine (and/or a pharmaceutically-acceptable salt, solvate, hydrate or prodrug thereof) for anti-cancer use, and which to exclude, optionally wherein almitrine (and/or a pharmaceutically-acceptable salt, solvate, hydrate or prodrug thereof) is administered in the trial in combination with another anti-cancer treatment(s) e.g. chemotherapy/chemotherapies and/or radiotherapy.
[10] The method of Claim 9, wherein those subjects whose cancer has
(i) a higher “GENE Q” protein(s) and/or “GENE Q” nucleic acid(s) amount, above a threshold amount, and/or
(ii) a lower “GENE Y” protein(s) and/or “GENE Y” nucleic acid(s) amount, below a threshold amount, are entered into a clinical trial of almitrine (and/or a pharmaceutically-acceptable salt, solvate, hydrate or prodrug thereof) administration for anti-cancer therapy, and those subjects whose cancer has
(iii) a lower “GENE Q” protein(s) and/or “GENE Q” nucleic acid(s) amount, below a threshold amount, and/or
(iv) a higher “GENE Y” protein(s) and/or “GENE Y” nucleic acid(s) amount, above a threshold amount, are not.
[11] The method of any one or more of Claims 1-10, wherein
the threshold amount for “GENE Q” is the mean/median/mode/decile/quartile/percentile, or some other function, of “GENE Q” protein(s) and/or “GENE Q” nucleic acid(s) amount in a normal and/or cancer derived sample(s) cohort; and/or
the threshold amount for “GENE Y” is the mean/median/mode/decile/quartile/percentile, or some other function, of “GENE Y” protein(s) and/or “GENE Y” nucleic acid(s) amount in a normal and/or cancer derived sample(s) cohort.
[12] The method of any one or more of Claims 1-11, wherein
the amount of “GENE Q” in the biological sample(s) from the subject (e.g. cancer sample) is normalized against the amount of one or more reference gene products in this biological sample(s) to obtain a normalized expression level of “GENE Q”, and wherein comparison is made against a standard value, or a set of standard values, that have been normalized by the same method; and/or
the amount of “GENE Y” in the biological sample(s) from the subject (e.g. cancer sample) is normalized against the amount of one or more reference gene products in the biological sample(s) to obtain a normalized expression level of “GENE Y”, and wherein comparison is made against a standard value, or a set of standard values, that have been normalized by the same method.
[13] The method of Claim 1, wherein the level of one or more “GENE Q” and/or “GENE Y” protein(s), and/or one or more “GENE Q” and/or “GENE Y” nucleic acid(s) (DNA/RNA), is measured ex vivo/in vitro in a sample(s) taken from said subject.
[14] The method according to Claim 1, wherein the sample(s) is derived from one or more of “liquid biopsy”, body wash (e.g. a lung wash sample), bodily fluid(s), tissue(s), normal tissue(s), cancer tissue(s), suspected cancer tissue(s), circulating cancer cells, cell line(s), urine, feces, plasma, serum and/or whole blood, or an extract or processed sample produced from any thereof.
[15] The method of Claim 1 wherein the sample comprises/contains cancer cells, and/or part(s) thereof, and/or nucleic acid and/or protein molecules thereof, from the subject's cancer/tumour.
[16] The method of Claim 1 wherein the subject is human; optionally elderly (e.g. >60 years old).
[17] The method of Claim 1, wherein the measuring is done by
(i) extracting RNA (optionally ˜1 nanogram) from a fixed, paraffin/wax-embedded cancer tissue sample obtained from the subject;
(ii) reverse transcribing RNA transcripts of one or more “GENE Q” (and/or “GENE Y”) to produce cDNA of “GENE Q” (and/or “GENE Y”);
(iii) amplifying the cDNA of one or more “GENE Q” (and/or “GENE Y”), optionally by polymerase chain reaction;
(iv) producing an amplicon of the RNA transcript of one or more “GENE Q” (and/or “GENE Y”);
(v) quantitatively assaying the level of the amplicon of the RNA transcript of one or more “GENE Q” (and/or “GENE Y”);
(vi) optionally normalizing the level of the amplicon of the RNA transcript of each “GENE Q” (and/or “GENE Y”) against a level of an amplicon of at least one reference RNA transcript in the tissue sample to provide, for each “GENE Q” (and/or “GENE Y”) assayed, a normalized “GENE Q” (and/or “GENE Y”) amplicon level.
[18] The method of Claim 1, wherein the measuring is done by a method of gene expression profiling.
[19] The method of Claim 1, wherein the measuring is done by a polymerase chain reaction (PCR)-based method.
[20] The method of Claim 1, wherein the measuring is done by a quantitative real time polymerase chain reaction (PCR)-based method.
[21] The method of Claim 20, wherein the real-time PCR method comprises forward and reverse primers.
[22] The method of Claim 1, wherein the measuring is done by quantitative reverse transcription polymerase chain reaction (qRT-PCR).
[23] The method of Claim 1, wherein the measuring is done by an array-based method.
[24] The method of Claim 1, wherein the measuring is done by a nucleic acid hybridization method comprising contacting said sample with a probe, wherein the sequence of the probe consists of a complimentary (base-pairing) nucleic acid sequence to all, or a component of, the mRNA or cDNA sequence of one or more of “GENE Q” and/or one or more of “GENE Y”, wherein the probe is attached to a solid substrate.
[25] The method of Claim 1, wherein the expression level is determined by a method selected from the group consisting of nucleic acid hybridization, nucleic acid amplification or a combination thereof.
[26] The method of Claim 24 or Claim 25, wherein the nucleic acid hybridization is performed using a solid-phase nucleic acid biochip array or in situ hybridization.
[27] The method of Claim 1, wherein the measuring is done by a sequencing-based method.
[28] The method of Claim 1, wherein the measuring is done by whole exome sequencing.
[29] The method of Claim 1, wherein the cancer sample comprises fragmented RNA.
[30] The method of Claim 1, wherein the cancer sample is obtained by biopsy or “liquid biopsy”.
[31] The method of Claim 1, wherein said sample is a tissue sample from fine needle, core, skin punch or other type of biopsy.
[32] The method of Claim 31, wherein the biopsy is a fine needle biopsy.
[33] The method of Claim 30, wherein the sample is a fine needle aspirate sample.
[34] The method of Claim 1, wherein the sample is a fresh or frozen fixed (e.g. formalin fixed), paraffin-embedded tissue (FPET) sample.
[35] The method of Claim 1, wherein the cancer sample is a fixed, paraffin-embedded tissue (FPET) sample.
[36] The method of Claim 1, wherein the cancer sample is frozen.
[37] The method of Claim 1, wherein the sample is a cytology sample, e.g. a thin layer cytological sample.
[38] The method of Claim 1, wherein the sample(s) from the subject is contacted with a specific binding agent, wherein the specific binding agent is selected from the group consisting of an antibody, a receptor, a lectin and a nucleic acid, which has specific binding affinity for a “GENE Q” (or “GENE Y”) protein product, wherein the specific binding agent and the “GENE Q” (or “GENE Y”) protein product form a binding agent-“GENE Q” protein complex (or a binding agent-“GENE Y” protein complex);
the amount of binding agent-“GENE Q” protein complex (or binding agent-“GENE Y” protein complex) formed is measured, thereby determining an amount of the “GENE Q” (or “GENE Y”) protein.
[39] The method of Claim 38, wherein the specific binding agent is an antibody.
[40] The method of Claim 1 comprising using a computer-implemented program (optionally implementing a statistical model[s]) to
(i) compare the one or more “GENE Q” protein(s) and/or “GENE Q” nucleic acid(s) amount in the subject's cancer with a reference value(s), and/or
(ii) compare the one or more “GENE Y” protein(s) and/or “GENE Y” nucleic acid(s) amount in the subject's cancer with a reference value(s),
to output a prediction (give an indication of; give a probabilistic indication of) of whether, optionally how much, the subject's cancer will be susceptible/responsive to almitrine (and/or a pharmaceutically-acceptable salt, solvate, hydrate or prodrug thereof) administration, optionally wherein a recommendation/instruction to, or recommendation/instruction not to, administer almitrine (and/or a pharmaceutically-acceptable salt, solvate, hydrate or prodrug thereof) to the subject is outputted.
[41] The method of Claim 1 wherein the predicted clinical response is expressed in terms of one or more of long-term survival, long-term survival without recurrence, Recurrence-Free Interval (RFI), Overall Survival (OS), Disease-Free Survival (DFS), or Distant Recurrence-Free Interval (DRFI).
[42] The method of Claim 1 with the added step of generating an oral/audio/written/electronically encoded/computer medium report (optionally accessible on a computer/phone/electronic device, optionally transmitted over the internet/intranet, optionally transmitted by electromagnetic radiation) based on the information, optionally information sourced from conducting the method of Claim 1 more than one time.
[43] The method of Claim 42, wherein the report comprises one or more of the following:
(i) information on prognosis, resistance, or potential therapeutic option(s);
(ii) information on the likely effectiveness of a therapeutic option(s);
(iii) the acceptability of a therapeutic option(s), or the advisability of applying/administering the therapeutic option(s) to the subject;
(iv) information on the administration of a drug(s);
(v) information to guide a cancer treatment decision for said subject;
(vi) whether, and/or how much, the subject's cancer is likely to be susceptible/responsive to treatment with almitrine (and/or a pharmaceutically-acceptable salt, solvate, hydrate or prodrug thereof) and/or with a recommendation/instruction to, or a recommendation/instruction not to, administer almitrine (and/or a pharmaceutically-acceptable salt, solvate, hydrate or prodrug thereof) to the subject.
[44] A method of requesting/instructing for the method of Claim 1, and optionally Claim 42, to be performed for a subject(s) with cancer and using the output to decide/determine (and/or as an input into deciding/determining) whether to administer almitrine (and/or a pharmaceutically-acceptable salt, solvate, hydrate or prodrug thereof) to the subject(s).
[45] The method of Claim 1 and/or Claim 44 wherein if/when the subject's cancer is diagnosed as being, or likely to be, almitrine sensitive/responsive then almitrine (and/or a pharmaceutically-acceptable salt, solvate, hydrate or prodrug thereof) is administered (preferably a therapeutically effective amount) to the subject.
[46] A method of requesting/instructing for the method of Claim 1, and optionally Claim 42, to be performed for a subject with cancer, and later administering a therapeutically effective amount of almitrine (and/or a pharmaceutically-acceptable salt, solvate, hydrate or prodrug thereof) to this subject.
[47] A kit for the method of Claim 1, i.e. predicting cancer response to almitrine (and/or a pharmaceutically-acceptable salt, solvate, hydrate or prodrug thereof), comprising reagent(s) necessary for
(i) measuring the level of one or more “GENE Q” protein(s) and/or “GENE Q” nucleic acid(s), and/or
(ii) measuring the level of one or more “GENE Y” protein(s) and/or “GENE Y” nucleic acid(s), in a biological sample(s) from a subject, further comprising a comparator module which comprises a standard value, or a set of standard values, to which the
(iii) level of one or more “GENE Q” protein(s) and/or “GENE Q” nucleic acid(s), and/or
(iv) level of one or more “GENE Y” protein(s) and/or “GENE Y” nucleic acid(s), in the sample is compared.
[48] A kit for predicting the cancer response to almitrine (and/or a pharmaceutically-acceptable salt, solvate, hydrate or prodrug thereof) comprising;
i) reagents for measuring a level of one or more “GENE Q” (and/or “GENE Y”) associated nucleic acid(s) (DNA/RNA) and/or protein(s) in a sample; and
ii) a comparator module.
[49] The kit according to Claim 44 and/or Claim 48, wherein the reagents comprise:
i) a capture reagent comprising a detector for one or more “GENE Q” (and/or “GENE Y”) protein product and
ii) a detection reagent.
[50] The kit according to Claim 49, wherein said capture reagent is an antibody.
[51] A method to predict the susceptibility/responsiveness of a subject's cancer to almitrine (and/or a pharmaceutically-acceptable salt, solvate, hydrate or prodrug thereof) treatment, comprising the steps of:
i) obtaining a biological sample(s) from the subject;
ii) determining a copy number of “GENE Q” in the test sample, optionally by Fluorescent In Situ Hybridization (FISH) (optionally with a fluorescent microscope or flow cytometer);
iii) comparing the copy number of “GENE Q” in the test sample against a normal copy number of “GENE Q”, wherein the normal copy number of “GENE Q” is two, thereby determining the presence or absence of a copy number change for “GENE Q” in the test sample;
iv) wherein the presence of increased copy number of “GENE Q” is associated with increased susceptibility/responsiveness to almitrine (and/or a pharmaceutically-acceptable salt, solvate, hydrate or prodrug thereof) anti-cancer treatment;
v) optionally, the finding(s) of the steps so far are included in an oral/audio/written/electronically encoded/computer medium report, optionally wherein, if/when there is an increased “GENE Q” copy number in the sample from the subject then the report recommends/instructs for almitrine (and/or a pharmaceutically-acceptable salt, solvate, hydrate or prodrug thereof) to be administered to the subject;
vi) optionally, if/when there is increased “GENE Q” copy number in the sample from the subject then almitrine (and/or a pharmaceutically-acceptable salt, solvate, hydrate or prodrug thereof) is administered (preferably a therapeutically effective amount) to the subject.
[52] The method according to Claim 51, but wherein it is performed for “GENE Y” instead, and where the presence of increased copy number of “GENE Y” is associated with decreased susceptibility/responsiveness to almitrine (and/or a pharmaceutically-acceptable salt, solvate, hydrate or prodrug thereof) anti-cancer treatment; wherein, if/when there is an increased “GENE Y” copy number in the sample from the subject then the report recommends/instructs for almitrine (and/or a pharmaceutically-acceptable salt, solvate, hydrate or prodrug thereof) not to be administered to the subject;
optionally, if/when there is increased “GENE Y” copy number in the sample from the subject then almitrine (and/or a pharmaceutically-acceptable salt, solvate, hydrate or prodrug thereof) is not administered (preferably a therapeutically effective amount) to the subject.
[53] The method according to Claim 51, wherein the sample(s) is derived from one or more of “liquid biopsy”, body wash (e.g. a lung wash sample), bodily fluid(s), tissue(s), normal tissue(s), cancer tissue(s), suspected cancer tissue(s), circulating cancer cells, cell line(s), urine, feces, plasma, serum and/or whole blood, or an extract or processed sample produced from any thereof.
[54] The method of Claim 51 wherein the subject is human; optionally elderly (e.g. >60 years old).
[55] The method of Claim 51, wherein the cancer sample is obtained by biopsy or “liquid biopsy”.
[56] The method of Claim 51, wherein said sample is a tissue sample from fine needle, core, skin punch or other type of biopsy.
[57] The method of Claim 51, wherein the biopsy is a fine needle biopsy.
[58] The method of Claim 51, wherein the sample is a fine needle aspirate sample.
[59] The method of Claim 51, wherein the sample is a cytology sample, e.g. a thin layer cytological sample.
[60] The method of Claim 51, wherein the “GENE Q” copy number change is caused by a deletion, amplification or translocation of a “GENE Q” gene or major part of it.
[61] The method of Claim 51, wherein the sample has an abnormally high copy number of “GENE Q” and overexpression of one or more of “GENE Q” and/or underexpression of one or more of “GENE Y”.
[62] A kit comprising tool(s) for detecting a “GENE Q” copy number change in a biological sample(s) and/or tool(s) for detecting overexpression of at least one “GENE Q” and/or underexpression of at least one “GENE Y” in a biological sample(s).
[63] A method of predicting (giving an indication of; giving a probabilistic indication of) whether a cancer, optionally inside a subject, is susceptible/responsive to almitrine (and/or a pharmaceutically-acceptable salt, solvate, hydrate or prodrug thereof) administration by comparing whether its gene/mRNA/cDNA/protein expression of one or more of “GENE Q” is equal or higher (and/or substantially similar), and/or its gene/mRNA/cDNA/protein expression of one or more of “GENE Y” is equal or lower (and/or substantially similar), than a cancer(s) (optionally a cancer cell line[s]) known to be susceptible/responsive to almitrine (and/or a pharmaceutically-acceptable salt, solvate, hydrate or prodrug thereof) administration.
[64] The method of Claim 63 wherein if/when a subject's cancer is predicted to be susceptible/responsive to almitrine (and/or a pharmaceutically-acceptable salt, solvate, hydrate or prodrug thereof) administration this subject is administered with a therapeutically effective amount of almitrine (and/or a pharmaceutically-acceptable salt, solvate, hydrate or prodrug thereof).
[65] A method of treating a subject (e.g. a human subject) diagnosed with cancer comprising administering almitrine (and/or a pharmaceutically-acceptable salt, solvate, hydrate or prodrug thereof) to the subject with a difference score determined from a cancer/tumor sample (optionally from a biopsy or “liquid biopsy”) from the subject that is above a cutoff value of the 50^{th} percentile or greater of the difference score in a reference population with the same diagnosis as the subject, wherein the difference score is the difference between a level of expression of one or more “GENE Q” (if/when more than one “GENE Q”, their mean is taken and used) and a level of expression of one or more “GENE Y” (if/when more than one “GENE Y”, their mean is taken and used).
[66] A method of treating a subject (e.g. a human subject) diagnosed with cancer comprising administering a therapeutically effective amount of almitrine (and/or a pharmaceutically-acceptable salt, solvate, hydrate or prodrug thereof) to the subject, wherein the subject's cancer has been predicted/determined to be responsive to almitrine (and/or a pharmaceutically-acceptable salt, solvate, hydrate or prodrug thereof) administration according to a method comprising:
(a) contacting a cancer/tumor sample (optionally from a biopsy or “liquid biopsy”) from the subject, comprising nucleic acid molecules, with a device (optionally a microarray) comprising:
(i) single-stranded nucleic acid molecules (optionally immobilized on a solid substrate) capable of specifically hybridizing with the nucleotides (e.g. mRNA and/or cDNA) of one or more of “GENE Q”; and
(ii) single-stranded nucleic acid molecules (optionally immobilized on a solid substrate) capable of specifically hybridizing with the nucleotides (e.g. mRNA and/or cDNA) of one or more of “GENE Y”; and
(b) detecting a level of expression of one or more of “GENE Q” and one or more of “GENE Y”; measuring hybridization between the one or more nucleic acid molecules from the sample and the single-stranded nucleic acid molecules of the device to detect a level of expression of one or more of “GENE Q” and one or more of “GENE Y”; and
(c) calculating a difference score for the subject's cancer by subtracting the level of expression of one or more of “GENE Y” (if/when more than one “GENE Y”, their mean is taken and used) from the level of expression of one or more of “GENE Q” (if/when more than one “GENE Q”, their mean is taken and used), wherein the difference score is above a cutoff value of the 50^{th} percentile or greater of the difference score in a reference population with the same diagnosis as the subject; and/or
(c) the level of expression of the “GENE Q” and “GENE Y” biomarkers in the cancer/tumor sample is equal to or above a cutoff difference level of expression of the “GENE Q” and “GENE Y” biomarkers in a cell or tissue known to be sensitive to the treatment with almitrine (and/or a pharmaceutically-acceptable salt, solvate, hydrate or prodrug thereof), wherein the cutoff difference level is obtained by subtracting the level of the “GENE Y” biomarker(s) (if/when more than one “GENE Y”, their mean is taken and used) from the level of the “GENE Q” (if/when more than one “GENE Y”, their mean is taken and used) biomarker(s).
[67] The method of Claim 66 where the (b) and (c) steps are instead:
(b) detecting a level of expression of the biomarkers of sensitivity (plurality of “GENE Q”) and the biomarkers of resistance (plurality of “GENE Y”); and
(c) calculating a difference score for the patient by subtracting a mean of the level of expression of the biomarkers of resistance from a mean of the level of expression of the biomarkers of sensitivity, wherein the difference score is above a cutoff value.
[68] The method of Claim 66 where the (c) step is substituted with a step whereby the subject's cancer is predicted/determined to be responsive to almitrine (and/or a pharmaceutically-acceptable salt, solvate, hydrate or prodrug thereof) if/when:
(i) the level of expression of one or more of “GENE Q” (biomarkers of sensitivity) is substantially similar to the level of expression of the same one or more of “GENE Q” in a cancer(s) known to be sensitive/responsive to almitrine (and/or a pharmaceutically-acceptable salt, solvate, hydrate or prodrug thereof) administration; and/or
(ii) the level of expression of one or more of “GENE Y” (biomarkers of resistance) is substantially similar to the level of expression of the same one or more of “GENE Y” in a cancer(s) known to be sensitive/responsive to almitrine (and/or a pharmaceutically-acceptable salt, solvate, hydrate or prodrug thereof) administration; and/or
(iii) the level of expression of one or more of “GENE Q” (biomarkers of sensitivity) is substantially dissimilar to the level of expression of the same one or more of “GENE Q” in a cancer(s) known to be resistant to almitrine (and/or a pharmaceutically-acceptable salt, solvate, hydrate or prodrug thereof) administration; and/or
(iv) the level of expression of one or more of “GENE Y” (biomarkers of resistance) is substantially dissimilar to the level of expression of the same one or more of “GENE Y” in a cancer(s) known to be resistant to almitrine (and/or a pharmaceutically-acceptable salt, solvate, hydrate or prodrug thereof) administration.
[69] The method of Claim 66, wherein:
(a) the device comprises at least two, or at least three, or at least four, or at least five, or at least six, or at least seven, or at least eight, or at least nine, or at least ten, or more single-stranded nucleic acid molecules of (i) and/or (ii);
(b) the one or more single-stranded nucleic acid molecules of the device have a length in the range of 10-100 (and/or 15-60, and/or 20-60) nucleotides;
(c) the level of expression of one or more of “GENE Q” and/or “GENE Y” is determined by one or more of microarray analysis, a nucleic acid amplification method(s), quantitative reverse transcriptase polymerase chain reaction (qRT-PCR), and/or qRT-loop-mediated isothermal amplification (LAMP).
[70] The method of one or more of Claim 65, Claim 66, Claim 67, wherein the cutoff value is at a 50^{th} percentile or greater of the difference score in a reference population with the same diagnosis as the subject.
[71] The method of one or more of Claim 65, Claim 66, Claim 67, wherein the cutoff value is at a 60^{th} percentile or greater of the difference score in a reference population with the same diagnosis as the subject.
[72] The method of one or more of Claim 65, Claim 66, Claim 67, wherein the cutoff value is at a 70^{th} percentile or greater of the difference score in a reference population with the same diagnosis as the subject.
[73] The method of one or more of Claim 65, Claim 66, Claim 67, wherein the cutoff value is at a 80^{th} percentile or greater of the difference score in a reference population with the same diagnosis as the subject.
[74] The method of one or more of Claim 65, Claim 66, Claim 67, wherein the cutoff value is at a 90^{th} percentile or greater of the difference score in a reference population with the same diagnosis as the subject.
[75] The method of one or more of Claim 65, Claim 66, Claim 67, Claims 70-74 wherein “reference population” refers to the cancers of this “reference population”.
[76] The method of one or more of Claim 65, Claim 66, Claim 67, wherein:
i) the difference score is substantially similar to a difference score of a subject's cancer (e.g. a human subject) known to be sensitive/responsive to almitrine (and/or a pharmaceutically-acceptable salt, solvate, hydrate or prodrug thereof) administration; and/or
ii) the difference score is substantially dissimilar to a difference score of a subject's cancer (e.g. a human subject) known to be resistant to almitrine (and/or a pharmaceutically-acceptable salt, solvate, hydrate or prodrug thereof) administration.
[77] The method of one or more of Claim 65, Claim 66, Claim 67, further comprising administering one or more cancer treatments/therapies other than almitrine (and/or a pharmaceutically-acceptable salt, solvate, hydrate or prodrug thereof) to the subject, wherein, optionally, the one or more cancer treatments/therapies are selected from surgery, radiation, and/or a (preferably FDA and/or EMA and/or MHRA and/or PMDA and/or NMPA approved) therapeutic agent(s).
[78] The method of one or more of Claim 65, Claim 66, Claim 67, wherein:
(a) if/when the level of expression of the at least one biomarker of resistance indicates that the patient is not resistant to the at least one treatment for cancer, said method further comprises administering the at least one treatment for cancer to the patient as a first cancer treatment; or
(b) if/when the level of expression of the at least one biomarker of resistance indicates that the patient is resistant to the at least one treatment for cancer, said method further comprises administering a second cancer treatment that is different from the first cancer treatment.
[79] The method of Claim 77, wherein said one or more additional therapies is administered:
(a) concurrently with said administration of almitrine (and/or a pharmaceutically-acceptable salt, solvate, hydrate or prodrug thereof); or
(b) separately from said administration of almitrine (and/or a pharmaceutically-acceptable salt, solvate, hydrate or prodrug thereof); or
(c) prior to said administration of almitrine (and/or a pharmaceutically-acceptable salt, solvate, hydrate or prodrug thereof); or
(d) after said administration of almitrine (and/or a pharmaceutically-acceptable salt, solvate, hydrate or prodrug thereof).
[80] A method wherein if/when the subject's cancer is not predicted (e.g. by a method or sub-method herein) to be susceptible/responsive to the administration of almitrine (and/or a pharmaceutically-acceptable salt, solvate, hydrate or prodrug thereof) then they are administered with almitrine (and/or a pharmaceutically-acceptable salt, solvate, hydrate or prodrug thereof) and one or more anti-cancer treatments/therapies, optionally selected from surgery, radiation, a (preferably FDA and/or EMA and/or MHRA and/or PMDA and/or NMPA approved) anti-cancer therapeutic agent(s).
[81] A method of treating a subject (e.g. a human subject) diagnosed with cancer comprising administering a therapeutically effective amount of almitrine (and/or a pharmaceutically-acceptable salt, solvate, hydrate or prodrug thereof) to the subject with a difference score determined from a cancer/tumor sample (optionally from a biopsy or “liquid biopsy”) from the subject;
wherein the difference score is substantially similar to a difference score of a subject's cancer (e.g. a human subject) known to be sensitive/responsive to almitrine (and/or a pharmaceutically-acceptable salt, solvate, hydrate or prodrug thereof) administration; and/or
wherein the difference score is substantially dissimilar to a difference score of a subject's cancer (e.g. a human subject) known to be resistant to almitrine (and/or a pharmaceutically-acceptable salt, solvate, hydrate or prodrug thereof) administration; and
wherein the difference score is the difference between a level of expression of one or more “GENE Q” (if/when more than one “GENE Q”, their mean is taken and used) and a level of expression of one or more “GENE Y” (if/when more than one “GENE Y”, their mean is taken and used);
optionally wherein the difference score is the difference between the level of expression of SCAF11 and the level of expression of RAPH1.
[82] A method of treating a subject (e.g. a human subject) diagnosed with cancer comprising administering a therapeutically effective amount of almitrine (and/or a pharmaceutically-acceptable salt, solvate, hydrate or prodrug thereof) to the subject, wherein the subject's cancer has been predicted/determined to be responsive to almitrine (and/or a pharmaceutically-acceptable salt, solvate, hydrate or prodrug thereof) administration according to a method comprising:
(a) contacting a cancer/tumor sample (optionally from a biopsy or “liquid biopsy”) from the subject, comprising nucleic acid molecules, with a device (optionally a microarray) comprising:
(i) single-stranded nucleic acid molecules (optionally immobilized on a solid substrate) capable of specifically hybridizing with the nucleotides (e.g. mRNA and/or cDNA) of one or more of “GENE Q”; and
(ii) single-stranded nucleic acid molecules (optionally immobilized on a solid substrate) capable of specifically hybridizing with the nucleotides (e.g. mRNA and/or cDNA) of one or more of “GENE Y”; and
(b) detecting a level of expression of one or more of “GENE Q” and one or more of “GENE Y”; measuring hybridization between the one or more nucleic acid molecules from the sample and the single-stranded nucleic acid molecules of the device to detect a level of expression of one or more of “GENE Q” and one or more of “GENE Y”; and
(c) calculating a difference score for the subject's cancer by subtracting the level of expression of one or more of “GENE Y” (if/when more than one “GENE Y”, their mean is taken and used) from the level of expression of one or more of “GENE Q” (if/when more than one “GENE Q”, their mean is taken and used); wherein the difference score is substantially similar to the difference score of a subject's cancer (e.g. a human subject) known to be sensitive/responsive to almitrine (and/or a pharmaceutically-acceptable salt, solvate, hydrate or prodrug thereof) administration; and/or
wherein the difference score is substantially dissimilar to the difference score of a subject's cancer (e.g. a human subject) known to be resistant to almitrine (and/or a pharmaceutically-acceptable salt, solvate, hydrate or prodrug thereof) administration;
optionally wherein the difference score is the difference between the level of expression of SCAF11 and the level of expression of RAPH1.
[83] The method of one or more of Claims 1-82, wherein the number of “GENE Q” (biomarkers of sensitivity) used is selected from at least 1, at least 2, at least 3, at least 4, at least 5, at least 6, at least 7, at least 8, at least 9, at least 10, at least 11, at least 12, at least 13, at least 14, at least 15, at least 16, at least 17, at least 19, at least 20, at least 30, at least 40, and/or wherein the number of “GENE Y” (biomarkers of resistance) used is selected from at least 1, at least 2, at least 3, at least 4, at least 5, at least 6, at least 7, at least 8, at least 9, at least 10, at least 11, at least 12, at least 13, at least 14, at least 15, at least 16, at least 17, at least 19, at least 20, at least 30, at least 40.
[84] The method of one or more of Claims 1-83, wherein:
(i) sensitivity of the cancer(s) known to be sensitive to almitrine (and/or a pharmaceutically-acceptable salt, solvate, hydrate or prodrug thereof) administration is based on GI50 data of NCI-60 cell lines; and/or
(ii) resistance of the cancer(s) known to be resistant to almitrine (and/or a pharmaceutically-acceptable salt, solvate, hydrate or prodrug thereof) administration is based on GI50 data of NCI-60 cell lines.
[85] The method of one or more of Claims 1-84, wherein almitrine (and/or a pharmaceutically-acceptable salt, solvate, hydrate or prodrug thereof) is administered to the subject intravenously and/or orally; optionally 50 to 500 mg almitrine dimesylate orally administered to the subject per day.
[86] A method of treating a subject (e.g. a human subject) diagnosed with cancer, wherein their cancer has high/overexpressed MYC protein and/or nucleic acid expression, optionally wherein this greater MYC amount confers (or is at least associated/correlated with its) resistance (or decreased susceptibility) to a cancer treatment(s)/drug(s) {non-limiting e.g. imatinib/dasatinib/nilotinib/radotinib/bosutinib/ponatinib, or a salt thereof}, optionally wherein the cancer is AML or CML or NSCLC, comprising administering a therapeutically effective amount of almitrine (and/or a pharmaceutically-acceptable salt, solvate, hydrate or prodrug thereof) to the subject, optionally in co-therapy with the aforementioned cancer treatment(s)/drug(s) in this claim, optionally conferring synergistic anti-cancer activity.

### Almitrine and Lactate

A high almitrine dose (intravenous delivery of 459±155 mg almitrine dimesylate, infused within 24 hours, which is a multiple of the (presently) clinically employed 50 to 200 mg oral daily dose) can potentially increase blood [lactate] in subjects with poor/impaired liver function; those with normal liver function are without any adverse effect [16]. Deselecting subjects with poor/impaired liver function from almitrine (and/or a pharmaceutically-acceptable salt, solvate, hydrate or prodrug thereof) administration is an optional embodiment(s) of this disclosure. A disclosure embodiment(s) is to find an individualized upper bound for almitrine (and/or a pharmaceutically-acceptable salt, solvate, hydrate or prodrug thereof) dose in a subject, wherein the almitrine dose (e.g. daily) is increased until there is a notable increase in the subject's plasma [lactate] from a baseline recorded prior to the course of administration, wherein the continuation dose is then set to be some function of this e.g. equal or a proportion (e.g. half) of it, wherein the almitrine dose arrived at is heavily dependent upon the subject's liver capacity which can be measured, e.g. by assaying plasma [bilirubin], prior to the course of almitrine treatment and the starting almitrine dose set accordingly, wherein those with poor liver function are administered a lower almitrine dose, or no almitrine at all.
[87] A method to predict/increase the safety of administering almitrine (and/or a pharmaceutically-acceptable salt, solvate, hydrate or prodrug thereof) to a subject, comprising the steps of:
i) obtain a blood/plasma sample(s) from the subject;
ii) measure plasma [bilirubin] and/or plasma [lactate];
iii) as determined by someone of the art (e.g. doctor/oncologist/clinician): if/when plasma [bilirubin], and/or plasma [lactate], is high then do not, or if/when it is within normal/expected/safe bounds for a subject with cancer (cancer can increase plasma [lactate]), do administer almitrine (and/or a pharmaceutically-acceptable salt, solvate, hydrate or prodrug thereof) to the subject;
iv) iterate the following one or more times: at an irregular/regular interval(s), non-limiting e.g. hourly/daily/every other day/every 3^{rd} day/weekly/fortnightly/monthly etc., during the course of almitrine administration, repeat steps (i) and (ii) and if/when plasma [bilirubin], and/or plasma [lactate], is high/unsafe/abnormal, and/or higher than prior, then optionally discontinue or lower the dose of almitrine (and/or a pharmaceutically-acceptable salt, solvate, hydrate or prodrug thereof) administration, or if/when it is within normal/expected/safe bounds, continue the course of almitrine (and/or a pharmaceutically-acceptable salt, solvate, hydrate or prodrug thereof) administration to the subject, optionally at a higher dose;
v) if/when upon conducting step (iv) for one or more iterations, plasma [bilirubin], and/or plasma [lactate], is observed to remain within normal/expected/safe bounds after/during almitrine administration, then almitrine (and/or a pharmaceutically-acceptable salt, solvate, hydrate or prodrug thereof) administration is continued without steps (i-iv).
[88] The method of Claim 87, wherein a different measure(s) of liver function is used, wherein multiple methods, and combination thereof, are known to those of the art.
[89] The method of Claim 87, wherein it is performed after one or more of Claims 1-85 has been conducted, preferably wherein one or more these methods has predicted that the subject's cancer will be susceptible/responsive to almitrine (and/or a pharmaceutically-acceptable salt, solvate, hydrate or prodrug thereof) administration.
[90] The method of one or more of Claims 1-86, only conducted for the subject if/when the following method of the present Claim, conducted prior, shows that almitrine (and/or a pharmaceutically-acceptable salt, solvate, hydrate or prodrug thereof) administration is safe/permissible/recommended in the subject, comprising the steps of:
i) obtain a blood/plasma sample(s) from the subject;
ii) measure plasma [bilirubin] and/or plasma [lactate];
iii) as determined by someone of the art (e.g. doctor/oncologist/clinician): if/when plasma [bilirubin], and/or plasma [lactate], is high then almitrine (and/or a pharmaceutically-acceptable salt, solvate, hydrate or prodrug thereof) administration is not recommended, or if/when it is within normal/expected/safe bounds for a subject with cancer (cancer can increase plasma [lactate]), then almitrine (and/or a pharmaceutically-acceptable salt, solvate, hydrate or prodrug thereof) administration is possible/permissible for the subject;
iv) optionally almitrine (and/or a pharmaceutically-acceptable salt, solvate, hydrate or prodrug thereof) is administered to the subject and the following is iterated one or more times: at an irregular/regular interval(s), non-limiting e.g. hourly/daily/every other day/every 3^{rd} day/weekly/fortnightly/monthly etc., during the course of almitrine administration, repeat steps (i) and (ii) and if/when plasma [bilirubin], and/or plasma [lactate], is high/unsafe/abnormal, and/or higher than prior, then optionally discontinue or lower the dose of almitrine (and/or a pharmaceutically-acceptable salt, solvate, hydrate or prodrug thereof) administration, or if/when it is within normal/expected/safe bounds, continue the course of almitrine (and/or a pharmaceutically-acceptable salt, solvate, hydrate or prodrug thereof) administration to the subject, optionally at a higher dose;
v) optionally, using information gathered from conducting one or more steps of (i-iv), one or more times, produce an oral/audio/written/electronically encoded/computer medium report(s) disclosing the result(s) generated, optionally concluding/recommending/instructing whether almitrine (and/or a pharmaceutically-acceptable salt, solvate, hydrate or prodrug thereof) administration is safe/permissible/recommended in the subject, and, optionally, if so at what dose;
vi) optionally, if/when one or more steps of (i-v) have shown almitrine (and/or a pharmaceutically-acceptable salt, solvate, hydrate or prodrug thereof) administration to be safe/permissible in the subject then a method(s) of one or more of Claims 1-86 is conducted.

In alternative embodiments, the ordering of one or more steps is different than presented here and/or one or more steps are omitted.

### Broader Case

A method to predict/utilize the susceptibility/responsiveness/resistance of a subject's cancer to almitrine treatment, disclosed prior, is a sub-method/example of a broader method disclosed herein/now:

A method to select a drug(s), optionally/preferably one or more of the named/specified drugs herein, optionally a licensed/approved cancer drug(s) by the FDA and/or EMA and/or MHRA and/or PMDA and/or NMPA (and/or a different country/jurisdiction in the world), to administer to a subject to treat/ameliorate/prevent/combat their cancer:

### [1] Foundation Phase, PHASE 1:

Detect/retrieve cancer gene expression (mRNA/cDNA/protein/combination thereof) correlation(s) (preferably wherein the correlation for each gene has been, or is, assayed multiple times) to susceptibility/resistance to the anti-cancer activity(s) of a drug(s).
For non-limiting example, when a drug has been tested in the NCI-60 protocol (preferably the 5-dose protocol), use the NCI COMPARE algorithm, e.g. as illustrated by example (without limitation) herein for almitrine dimesylate. Submitting a compound(s)/drug(s) to NCI-60 testing for this purpose/method, and/or in use with one or more steps of this method, is componentry to this disclosure.
Contemplated by some embodiments is to use data of the drug's anti-cancer activity against different cancer cell lines (non-restrictive e.g. those cancer cell lines used in the NCI-60 screen, preferably using 57 cancer cell lines or greater) and the NCI COMPARE algorithm [6-7], or a variant/equivalent thereof, with at least one gene expression database, which comprises/contains the used cancer cell line's gene expression (optionally replicating/approximating the method, or variant thereof used herein, illustrated for almitrine dimesylate, which used services conferred by the National Cancer Institute [NCI]. But with a different compound(s)/drug(s). Optionally without direct use of NCI services. Wherein gene expression data for some cancer cell lines, including the cancer cell lines used in the NCI-60 screen, can be sourced from the CellMiner database [3-4] and/or from one or more source journal papers, and/or their supplementary information/data/appendix, used to populate this database. At least some of the gene expression databases used by the NCI COMPARE online service at the NCI are publically available, e.g. refer [17]. And the NCI COMPARE algorithm is publically available [6-7]).
Rank the cancer gene expression correlations (e.g. Pearson correlation coefficients) to drug(s) susceptibility/resistance by the number of times, n, that a gene expression is observed correlated above a desired (e.g. Pearson) correlation coefficient (R) cutoff value, x (|R|>x), to drug(s) susceptibility/resistance. And set another cutoff for number of times observed, y, at or above which a gene(s) expression is included in the prediction model.
Preferably, if/when the same direction of correlation to drug(s) susceptibility/resistance (positive or negative) is not observed for a given gene expression in all observations, optionally at |R|>x, then this gene is excluded from the prediction model.
In non-limiting embodiment examples, x is selected from 0.3, 0.35, 0.4, 0.45, 0.5, 0.55, 0.6, 0.65, 0.7, 0.75, 0.8, 0.85, 0.9, 0.95. And y is selected from an integer between 1 and 100, optionally an integer between 1 and 20. In non-limiting embodiment examples, x is selected from one or more of the following ranges (or a sub-range thereof) 0.3-0.4, 0.4-0.5, 0.5-0.6, 0.6-0.7, 0.8-0.9, 0.9-1. And y is selected from an integer between 1 and 100, optionally an integer between 1 and 20. In non-limiting embodiment examples, x and y values are set such that the number of gene expression biomarkers in the prediction model for the drug is one of the following numbers: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or a greater integer, optionally less than 1,000, optionally less than 100, optionally less than 10, optionally less than 5, optionally less than 3.
So, distinctively with this method, conceivably a gene expression correlation with a lower value of |R| can be included in the prediction model ahead of, optionally excluding (if/when a limited gene number is permitted in the prediction model), another gene expression correlation with a higher value of |R|, if/when the former correlation is observed more times. Conceivably, in this method, a gene expression correlation that is only observed once/few, even if/when with a very high |R| value(s), is given lower to no credence as compared to another gene expression correlation(s) observed multiple/many times with lower |R| values, even if/when much lower, so long as these latter |R| values are still above the cutoff value of |R|, x, and are all of same sign. In present methods of the art, x is considered important without sufficient consideration of y. By the teaching of this disclosure, the importance of y is recognized and incorporated. Given the “noisiness” of biology, signal replication is more indicative of true signal amplitude than is presently recognised by those of the art.
Using this method, the ranking of gene expression correlations is heavily dependent upon what cut-off value of x is selected, wherein this yields the enduring importance of x in this method.
Each gene(s) selected to be in the prediction model (by the selected cut-off settings of x and y), whose expression positively correlates with the drug's anti-cancer activity, can be termed “GENE Q”. And each gene(s) selected to be in the prediction model, whose expression negatively correlates with the drug's anti-cancer activity, can be termed “GENE Y”.
Moreover, by further teaching of this disclosure, if/when more than one gene expression meets the cut-off criteria set for x and y, optionally combine some or all of these selected gene expressions using, optionally more than once, one or more of the formulae for “GENE Q” and/or “GENE Y” given herein, to confer further possible embodiments of “GENE Q” and/or “GENE Y” for optional use in building the prediction model.
Optionally, cut-off values of x and y are independently selected for each drug of interest such that for each drug of interest there is less than twenty (or less than ten, or less than five) of each of “GENE Q” and “GENE Y” in the prediction model, optionally just one “GENE Q” and just one “GENE Y”, and independently for each drug of interest these are then used in the equation: “DIFFERENTIAL”=(mean of “GENE Q”)−(mean of “GENE Y”)={(Σ^{a}GENE Q)/a}−1{(Σ^{b}GENE Y)/b}. Such that each drug of interest has its own “DIFFERENTIAL” value. In some embodiments, “DIFFERENTIAL” can also be referred to as “GENE Q”.
Using this selected data,
select a cutoff/reference/standard value(s) of “DIFFERENTIAL”: value above which is observed to generally correspond with a desired (e.g. high; non-limiting e.g. >50% {and/or >60% and/or >70% and/or >80% and/or >90%} cancer growth inhibition {and/or desired level of cancer cell killing} in NCI-60 one-dose and/or five-dose testing) level of drug(s) anti-cancer activity, and/or
select a cutoff/reference/standard value(s) of “GENE Q”: value of expression above which is observed to generally correspond with a desired (e.g. high; non-limiting e.g. >50% {and/or >60% and/or >70% and/or >80% and/or >90%} cancer growth inhibition {and/or desired level of cancer cell killing} in NCI-60 one-dose and/or five-dose testing) level of drug(s) anti-cancer activity, and/or
select a cutoff/reference/standard value(s) of “GENE Y”, value of expression below which is observed to generally correspond with a desired (e.g. high; non-limiting e.g. >50% {and/or >60% and/or >70% and/or >80% and/or >90%} cancer growth inhibition {and/or desired level of cancer cell killing} in NCI-60 one-dose and/or five-dose testing) level of drug(s) anti-cancer activity;
this reference value(s) can optionally be normalized, e.g. to the expression level of a chosen gene(s), e.g. to one or more (e.g. averaged) “housekeeping genes”;
this reference value(s), normalized or not (as desired), is then used in PHASE (2), which is disclosed later.
The aforementioned can be repeated for more than one drug such that a cutoff/reference/standard value(s) for
one or more “DIFFERENTIAL”, and/or
one or more “GENE Q”,
and/or one or more “GENE Y”,
can be collected for a number of drugs (optionally all approved cancer drugs in a country/jurisdiction/continent, e.g. USA) and this method in its entirety can be used to select which is likely to be the best drug(s) to administer to a subject for their cancer, preferably wherein this selected drug(s), preferably a therapeutically effective amount, is then administered to the subject. So, this method yields personalized/tailored anti-cancer treatment.
In an embodiment(s), this method is used to select, as total or in part (e.g. along with selection by one or more of age, sex, cancer stage, prior treatment(s), life expectancy, present prognosis etc.—many further criteria are known to those of the art) selection criteria, which subject(s) to enter into a clinical trial of a drug(s), and which subject(s) to exclude. In some embodiments this method is used to stratify subjects into different treatment arms of a clinical/master trial, e.g. wherein subjects in different arms are administered a different drug(s)/treatment(s).
In some embodiments, cut-off values of x and y are independently selected for each drug of interest, independently set sufficiently high in each case, such that for each drug of interest there is just one “GENE Q” and just one “GENE Y” (those of each with the highest value of y), and so for each drug of interest “DIFFERENTIAL”=“GENE Q” minus “GENE Y”. Optionally, wherein if this method selects a “GENE Q” that is an already known (in the state of the art) biomarker for cancer susceptibility to this drug, then specifically for “GENE Q”, the stringency of cut-off x is iteratively decreased (e.g. by 0.1 each step, preferably not going lower than cut-off value of x=0.3), and the stringency of cut-off y is kept constant (or, optionally, if cut-off value of x=0.3, iteratively increased by 1), until an alternative “GENE Q” (which isn't known to correlate with cancer susceptibility to this drug in the state of the art) is selected by the selected cut-off values of x and y. Wherein the same method component is performed if this method selects a “GENE Y” that is an already known (in the state of the art) biomarker for cancer susceptibility for this drug.
In a non-limiting embodiment(s) example, the cut-off value of x is set at (or around) 0.3 {or, if using NCI COMPARE on the NCI website, sufficiently high that the number of results returned is no greater than the maximum possible to list, which is presently 2,000} and the cut-off value of y is floated (but always a positive integer, preferably iteratively increased, by 1 each step, from 1) depending upon how many biomarkers are desired in the prediction model, optionally wherein cut-off value of y is independently set (for selecting at least one “GENE Q”, and/or at least “GENE Y”) at a value, such that there are less than 10, or less than 9, or less than 8, or less than 7, or less than 6, or less than 5, or less than 4, or less than 3, or less than 2, or just 1 of “GENE Q” and/or “GENE Y”, optionally wherein the corresponding “DIFFERENTIAL” value is calculated, and utilized (e.g. by a method of using a “DIFFERENTIAL” value as taught by this disclosure). In this embodiment(s), with such a low cut-off value of x, emphasising y, where consideration of y is a distinctive aspect to this disclosure, this method embodiment(s) returns/outputs different biomarkers than are in the state of the art, even for well known, approved/licensed cancer drugs. These biomarkers, and use thereof (e.g. for determining whether to administer the drug to a subject with cancer, or not; wherein if determined in the affirmative, a therapeutically effective amount of the drug is administered to the subject), are componentry to this disclosure.
A method (optionally computer implemented) to find/retrieve a cancer gene expression biomarker(s) for susceptibility/resistance to a drug comprising selecting a gene whose expression is observed, more than y number of times, to correlate, with an absolute Pearson correlation coefficient (|R|) greater than x, with this drug's anti-cancer activity. Wherein in some non-limiting embodiments x is between 0.3 and 0.45, and y is 5, or 6, or 7, or 8, or 9, or 10, or 11, or 12, or 13, or 14, or 15, or 16, or 17, or 18, or 19, or 20. Distinctively, this method doesn't necessarily select the gene with the greatest value of |R|, but the gene(s) with >y number of observations above a relatively modest threshold value of |R|, x. And preferably retrieved gene(s) are grouped into “GENE Q” (expression level in cancer positively correlates with cancer susceptibility to drug) and/or “GENE Y” (expression level in cancer negatively correlates with cancer susceptibility to drug) categories. Optionally wherein their absolute value(s) and/or differential in expression is utilized, e.g. optionally by utilizing a formula herein, to predict/diagnose if a subject's cancer is likely to be susceptible to a cancer drug, optionally wherein if the subject's cancer is predicted to be susceptible, a therapeutically effective amount of this drug is administered to the subject.
A method (optionally computer implemented) to find/retrieve a cancer gene expression biomarker(s) for susceptibility/resistance to a drug comprising selecting a gene whose expression is observed to correlate with this drug's anti-cancer activity, with the restriction that this correlation must be observed multiple times, preferably above a cut-off number of times, wherein this cut-off can be, in some embodiments, selected from 4,5,6,7,8,9,10,11,12,13,14,15,16,17,18,19,20.

### [2] Subject Phase, PHASE 2:

i) obtain (or start with) a biological sample(s) from the subject;
ii) determine the DNA and/or mRNA and/or cDNA and/or protein expression level of one or more of “GENE Q” and/or one or more of “GENE Y” (relating to at least one drug) in said sample(s):
measuring the level of one or more of “GENE Q” and/or “GENE Y” protein(s) and/or one or more of “GENE Q” and/or “GENE Y” nucleic acid(s) (DNA/RNA) in a sample(s) obtained from the subject to obtain a value, or values, representing this level;
iii) optionally “GENE Q” and/or “GENE Y” value(s) is independently normalized to the expression (mRNA/cDNA/protein/combination thereof) value of a reference gene(s), e.g. one or more (e.g. averaged) “housekeeping genes”;
iv) compare said “GENE Q” and/or “GENE Y” value(s) (and/or a “DIFFERENTIAL” value(s) thereof) to a reference value(s) from PHASE (1):
comparing the value, or values, of the level(s) from step (ii) and/or step (iii) with a standard/reference value, or a set of standard/reference values, wherein this comparison can predict/diagnose the subject's cancer's susceptibility/responsiveness to a drug(s);
v) optionally repeat/iterate steps (ii), (iii) and (iv) for a different “GENE Q” and/or “GENE Y”;
vi) optionally combine multiple “GENE Q” and/or “GENE Y” values for a particular drug into a single “GENE Q” and/or “GENE Y” value for the drug using, optionally more than once, one or more of the formulae for “GENE Q” and/or “GENE Y” herein;

optionally calculate “DIFFERENTIAL”={(Σ^{a}GENE Q)/a}−{(Σ^{b}GENE Y)/b};
vii) optionally, using information gathered from running all or some steps (i-vi) one or more times, produce an oral/audio/written/electronically encoded/computer medium report(s) disclosing the result(s) generated, optionally concluding whether the subject's cancer is likely to be susceptible/responsive to a drug(s), optionally wherein this report(s) includes a recommendation/instruction/direction to administer, or not administer, a drug(s);
viii) optionally, request/order the report(s) referred to in step (vii) and/or, optionally only inherently, the conduction of one or more steps (i-vii);
ix) optionally, using information gathered from conducting one or more steps of (i-viii) one or more times, if/when
the value(s) of one or more of “GENE Q” (if/when more than one “GENE Q”, their mean can be taken and used) is above a threshold reference value (or a set of reference values), and/or
the value(s) of one or more of “GENE Y” (if/when more than one “GENE Y”, their mean can be taken and used) is below a threshold reference value (or a set of reference values), and/or
the value(s) of “DIFFERENTIAL” is above a threshold reference value (or a set of reference values), then the drug(s) that this reference value(s) relates to is administered (preferably a therapeutically effective amount) to the subject.

In alternative embodiments the ordering of one or more steps is different than presented here, and/or one or more steps are omitted. Step (i) of PHASE (2) can be omitted such that “said sample(s)” in step (ii) is then replaced with “a sample(s) obtained from a subject”: this alternative is better before the European Patent Office (EPO). It is componentry to this disclosure to only conduct one or more of the steps and not all in their entirety. One salient stand-alone sub-set is PHASE (2), steps (i-vii), which could be performed by a contract testing laboratory to a hospital for example, or steps (viii-ix) that could be performed by a doctor/oncologist for example. All the steps in entirety could be performed by a clinical trial program for example, e.g. by a Master trial protocol such as used by the Beat AML trial [12].

Using the method, or only part(s)/step(s) thereof, with a FDA/EMA/MHRA/PMDA/NMPA approved drug, optionally but not restrictively licensed for anti-cancer use, is componentry to the disclosure.

In this method, gene expression can be assayed at one or more of the mRNA, cDNA and protein levels, optionally with their data pooled to give an amalgamated value for each gene expression. In an alternative embodiment(s), the method is applied with microRNA and/or non-protein coding RNA sequence(s) instead of, or in addition to, gene expression. Some of the cutoffs in the method have been expressed in terms of greater than, or less than, but instead, at one or more places, a cutoff(s) in terms of equal or greater than, or equal or less than, is also contemplated by, and componentry to, the disclosure.

In a method embodiment(s), the level of one or more of “GENE Q” and/or “GENE Y” protein(s), and/or one or more of “GENE Q” and/or “GENE Y” nucleic acid(s) (DNA/RNA), is measured ex vivo/in vitro in a sample(s) taken from said subject. Wherein the sample(s) can be derived (without limitation) from one or more of “liquid biopsy”, body wash (e.g. a lung wash sample), bodily fluid(s), tissue(s), normal tissue(s), cancer tissue(s), suspected cancer tissue(s), circulating cancer cells, cell line(s), urine, feces, plasma, serum and/or whole blood, or an extract or processed sample produced from any thereof.

How to determine the amount of a DNA/RNA sequence(s), and/or an mRNA transcript(s), and/or protein(s), in a biological sample is well known to those of the art. And all such methods are contemplated by, and componentry to, this disclosure.

In an embodiment(s), step (ii) is replaced with, or added to, a step assaying for gene(s) amplification, e.g. detected (or lack thereof) by Fluorescent In Situ Hybridization (FISH), wherein an alternative step (iii) is to take a gene amplification to be synonymous with overexpression of that gene(s) and, optionally, a cancer with this amplification is then assumed to have above the threshold reference value(s) for this gene in step (iv). But wherein, in an alternative embodiment(s), this assumption is tested by conducting the original step (ii) for this gene, and proceeding with following steps accordingly.
An optional disclosure embodiment(s) is to manipulate the cut-off x and cut-off y parameters to select a desired number of gene expression correlations to a drug(s) activity. Wherein this selection is likely to be the best gene expression correlations available from the data available for that desired number of gene expression correlations.
A disclosure embodiment(s) is to set cut-off x at a reasonably high magnitude and then rank gene expression to drug(s) activity correlations, with |R|>x, on the basis of which has the most number of observations rather than which has highest value of R|, wherein those with conflicting correlations (e.g. both positive and negative observed at |R|>x) are disregarded, and wherein if/when number of observations is equal then the correlation with higher |R| is ranked higher.
In alternative embodiments this ranking is more nuanced, whereupon a correlation with less observations can be placed higher if/when its |R| value is much higher, especially if/when the difference in observations is small and/or the number of observations of the one with less, although less, is still substantial.
An optional disclosure embodiment(s) is to set cut-off x at a value such that |R|>x is significant at [p-value <z], where z can be 0.05 or some other significance threshold of the art, e.g. optionally a Bonferroni correction of 0.05: i.e. 0.05/number of genes for which expression is measured in the study.
In a variant embodiment(s) that can be substituted into the method, into PHASE (1), in the ranking of cancer gene expressions, rank cancer gene expression correlations to drug(s) susceptibility/resistance by how many times a correlation for a cancer gene expression is observed, and at what amplitude, by summing the |R| values, restricting to those above the cutoff value, x (|R|>x), for each cancer gene expression and ordering by which has greater summed |R| value, d, optionally wherein only gene expressions with a value of d above a selected cutoff, r, are included in the gene expression vs. cancer prediction model. Or alternatively only the selected top number by this method, v, of genes expressed is entered. This method variant simultaneously captures the dual importance of amplitude, and frequency of observation, of a gene expression correlation.
In some embodiments, cut-off values of x and r are independently selected for each drug of interest, independently set sufficiently high in each case, such that for each drug of interest there is just one “GENE Q” and just one “GENE Y” (those of each with the highest value of d), and so for each drug of interest “DIFFERENTIAL”=“GENE Q” minus “GENE Y”, wherein if this method selects a “GENE Q” that is an already known (in the state of the art) biomarker for cancer susceptibility to this drug, then specifically for “GENE Q”, the stringency of cut-off x is iteratively decreased (e.g. by 0.1 each step, preferably not going lower than cut-off value of x=0.3), and the stringency of cut-off value of r is kept constant (or, optionally, iteratively increased by 0.1 each step, or every other step, or every 5 steps, or every 10 steps, or some other interval of step), until an alternative “GENE Q” (which isn't known to correlate with cancer susceptibility to this drug in the state of the art) is selected by the selected cut-off values of x and r, wherein the same method component is performed if this method selects a “GENE Y” that is an already known (in the state of the art) biomarker for cancer susceptibility for this drug.
In further variants, when summing the |R| values for each gene expression correlation then |R| values above different threshold values can be ascribed different weighting in the summation by combining them with a constant (e.g. multiplicative constant, and/or addition of a constant etc.), wherein a different constant can be used depending on the highest threshold value of |R| that this |R| value is in excess of, before they are included in the summation, wherein over- and under-weighting can be used, but in a preferred embodiment(s) overweighting is favoured for higher |R| values, e.g. greater weighting constant used the higher the highest threshold value of |R| that this |R| value is in excess of.

In an embodiment(s), a correction(s) is applied if/when the same number of gene expression probes for each gene is not present in the gene expression database(s). For non-limiting example, a proportionally lower observation cutoff is used for a gene(s) with less probes. If/when the |R| summation method is used, |R| summation for a gene with less probes is increased by multiplication with a constant proportional to the probe number disparity between this gene and another(s), which it is compared to; alternatively |R| summation for a gene with more probes is decreased by division with a constant proportional to the probe number disparity between this gene and another(s), which it is compared to, wherein one or both these methods, optionally with different constants for different genes as applicable to probe number disparit[y/ies], is used to enable fair comparison across all the gene expressions considered.

Componentry to this disclosure is for one or more of these method steps, or for any one or more of the method steps herein, disclosed by this disclosure, to be implemented by a computer operation(s)/program(s), optionally a phone app. At any place that “method” is written/implied herein, in alternative embodiments this is substituted with “computer implemented method”. Any method herein (or sub-method(s) thereof), implemented by a computer, is componentry to this disclosure.

A kit(s) for implementing all or part of this method(s)—a kit(s) to select a drug(s) to treat/ameliorate/prevent/combat a subject's cancer, and/or a kit(s) to predict/diagnose the susceptibility/responsiveness/resistance of a subject's cancer to a drug(s)—is componentry to this disclosure, wherein a kit for implementing PHASE (2) is especially preferred comprising, optionally equipment(s) to draw a biological sample(s) from the subject, reagent(s) necessary for measuring the level of one or more “GENE Q” and/or “GENE Y” protein/DNA/cDNA/RNA/mRNA in a biological sample(s) from a subject, further comprising comparator information/module which comprises (optionally derived from PHASE (1)) a standard value, or a set of standard values, to which the level of “GENE Q” and/or “GENE Y” protein/DNA/cDNA/RNA/mRNA in the sample is compared, optionally with instruction(s)/direction(s) for which drug(s) to administer on the basis of the comparison. Optionally the comparator module is a computer and/or software, optionally which can be updated, optionally by information/data (optionally encrypted) sent across the internet, which has the benefit that the standard/threshold value(s)/recommendation(s)/direction(s) can be updated as further drug(s) are investigated/developed. Optionally wherein the PHASE (1) method step is iterated, optionally in a systematic program, to generate further data that can be used to update the comparator module, optionally wherein a subscription is required to receive an information update(s).

In a disclosure embodiment(s), PHASE (1) of the method is conducted with human subjects, e.g. using a sample(s) drawn from each subject, from which it is observed, across different subjects (more is better), which gene(s) expression in a subject's cancer correlates with observed anti-cancer activity in the subject (and/or in a sample of the subject's cancer grown in vitro and/or grown in an animal/mouse avatar), or lack thereof (resistance), to a drug(s). Optionally wherein the subjects are in a clinical trial(s), e.g. the BEAT AML trial [12]. Optionally wherein the subject(s) of PHASE (2) of the method are in the same trial or a daughter of it. In this way, PHASE (2) can become PHASE (1) for a later PHASE (2), optionally iterated, and so the method iteratively becomes better.

In a disclosure embodiment(s), PHASE (1) of the method is the BEAT AML trial [12] (itself or its protocol {or variant thereof} replicated/modified) wherein its output data upon some subjects being administered almitrine (and/or a pharmaceutically-acceptable salt, solvate, hydrate or prodrug thereof) is used with a method herein, or other method (e.g. as in [12]), to find cancer gene expression (and/or gene mutation(s) and/or cytogenetic abnormalit[y/ies]) correlation(s) to susceptibility to the anti-cancer activity of almitrine (and/or a pharmaceutically-acceptable salt, solvate, hydrate or prodrug thereof), optionally which is then used to select some, and exclude other, further subjects to administer almitrine (and/or a pharmaceutically-acceptable salt, solvate, hydrate or prodrug thereof) for anti-cancer treatment.

Using a biomarker(s) (non-limiting e.g. gene(s) expression, mutation(s), SNP(s), epigenetic/methylation marker(s), cytogenetic abnormalit[y/ies], metabolite(s) level, enzyme(s) activit[y/ies], antigen(s), cell surface protein(s), genomic/transcriptomic/proteomic/metabolomic marker(s), cell-free DNA(s) etc.) measured in a subject, and/or in a biological sample(s) sourced from a subject, to decide/determine (and/or as an input into deciding/determining) whether to administer almitrine (and/or a pharmaceutically-acceptable salt, solvate, hydrate or prodrug thereof) to treat/ameliorate/prevent/combat cancer in the subject is componentry to this disclosure.

At any point that almitrine is mentioned/implied herein, in alternative embodiments this mention/implication is substituted with a different drug(s) mentioned/specified herein, and correspondingly—in each case—the mentions of “GENE Q” and “GENE Y” and “DIFFERENTIAL” herein are substituted for those applicable/taught for this other drug(s).

Any method used/disclosed herein to source and select cancer gene expression correlations to almitrine dimesylate susceptibility/resistance (biomarkers), for incorporation into a prediction model of cancer susceptibility/resistance to almitrine dimesylate, is contemplated with a different drug mentioned/specified herein, for generating a prediction model of susceptibility/resistance to that drug. And the resulting correlations (biomarkers) and prediction models are hereby disclosed herein. And the administration of a therapeutically effective amount of a drug(s) to a subject, by following/using such a prediction model, is herein contemplated and componentry to this disclosure.

### Illustrating Example of PHASE I of the Aforementioned Method

Acquiring cancer gene expression biomarkers for susceptibility/resistance to Ibrutinib drug (NSC number: 761910). Went to https://dtp.cancer.gov/pubic_compare/ and inputted its NSC number, in the box labelled for inputting the NSC number. Clicked search. Then, in the page thereafter, clicked the circle with C inside of it (the symbol for NCI COMPARE). Then, in the page thereafter, for “target set name”, I selected “MOLTID_GC_SERIES_MICROARRAY_ALL”, which, as aforementioned, is a favoured set/collection of a number of cancer gene expression databases. For count of results to return I selected the maximum, which is presently 2,000. For minimum correlation I selected the smallest minimum correlation value, at or above 0.3, that returned less than the maximum number of returnable results in the system (which is presently 2,000). Which was found by trial and error to be 0.4. Thereafter I retrieved the results (with minimum correlation=0.4), of cancer gene expression correlations to the drug's anti-cancer activity (in NCI-60 testing), downloading them in Microsoft Excel format. There were 1,423 entries. I then searched for those genes that were present the most times in these results. By finding how many times each gene present was observed in this data set, and ranking them, from most frequently observed to least. Then setting a cut-off number of observations, above which the gene was included in the prediction model, and below which it wasn't. Wherein a different cut-off was used for those genes whose expression correlates positively, and for those whose expression correlates negatively. The cut-off number selected in each case respectively determining how many “GENE Q” (gene whose expression positively correlates with drug's anti-cancer activity) and “GENE Y” (gene whose expression negatively correlates with drug's anti-cancer activity) I pulled from this data, to use in my prediction model. In this example, I used high cut-offs such that only a very small number of “GENE Q” and/or “GENE Y” were retrieved. Which, because of their high frequency of observation, are likely to be incredibly robust. These are presented below. This method is very distinctive from what one of the art would do: they would look to see which gene(s) had the highest correlation R score and build a prediction model using this/these, without any consideration of frequency of observation. They would select for highest value of R, instead of selecting for highest frequency of observation over a modest threshold value of R. Probably not even seeing all these results because when running NCI COMPARE they would set the minimum correlation threshold as high as they could, so long as it still returned results. And utilize these genes returned. Possibly searching therein for the particular gene(s) with highest correlation value of R, and using this gene(s). They wouldn't set the minimum correlation as low as I did, such that 1,423 results are returned. A huge number. If the minimum correlation is instead set to 0.6, just 32 results are returned. If the minimum correlation is instead set to 0.65, just 2 results are returned (only one of which is found below: PDZK1IP1, which is only the 12^{th} ranked gene by this present method).
“GENE Q”: MACF1 (correlation score, R, greater than +0.4 observed 16 times).
   PARD3 (correlation score, R, greater than +0.4 observed 15 times).
   PDXK (correlation score, R, greater than +0.4 observed 10 times).
   CD24 (correlation score, R, greater than +0.4 observed 10 times).
   ACTG1, TMEM56, CDR2L, GLRB, COL18A1, FAM45A, GLRB (correlation score, R, greater than +0.4 observed 8 times each).
   PDZK1IP1, TLDC1, PDGFC, FGF2 (correlation score, R, greater than +0.4 observed 7 times each).
   RRAS2 (correlation score, R, greater than +0.4 observed 6 times).
“GENE Y”: VPRBP (correlation score, R, greater than |-0.41 observed 7 times).
   C1QBP (correlation score, R, greater than |-0.41 observed 6 times).

In some embodiments, “DIFFERENTIAL”=MACF1−VPRBP, or “DIFFERENTIAL”=MACF1−PARD3, or “DIFFERENTIAL”={mean(MACF1,PARD3)}−VPRBP, or “DIFFERENTIAL”={mean(MACF1,PARD3,PDXK,CD24)}−{mean(VPRBP, C1QBP)}. In some embodiments, “GENE Q” is mean(MACF1,PARD3). In some embodiments, only “GENE Q” is used in the prediction model, and not “GENE Y”.

### Illustrating Example of PHASE I of the Aforementioned Method

Acquiring cancer gene expression biomarkers for susceptibility/resistance to Osimertinib drug (NSC number: 779217). Used the method as above. Except that in this case the smallest minimum correlation, at or above 0.3, which returned less than the maximum number of returnable results in the system (which is presently 2,000) was 0.35. With this, 1640 results returned.
“GENE Q”: ERBB2 (correlation score, R, greater than +0.35 observed 12 times).
   PDXK (correlation score, R, greater than +0.35 observed 8 times).
   THRB, TMEM56, PDZK1IP1, PAX8 (correlation score, R, greater than +0.35 observed 7 times each).
“GENE Y”: ANKH (correlation score, R, greater than |-0.351 observed 4 times).
   STX12 (correlation score, R, greater than |-0.351 observed 4 times).

### Illustrating Example of PHASE I of the Aforementioned Method

Acquiring cancer gene expression biomarkers for susceptibility/resistance to Palbociclib drug (NSC number: 758247). Used the method as above. Except that in this case, the smallest minimum correlation, at or above 0.3, which returned less than the maximum number of returnable results in the system (which is presently 2,000) was 0.45. With this, 1785 results returned.
“GENE Q”: GTF3A (correlation score, R, greater than +0.45 observed 11 times).
   MYC (correlation score, R, greater than +0.45 observed 9 times).
   KARS (correlation score, R, greater than +0.45 observed 9 times).
   MST4, ELF1, TPT1 (correlation score, R, greater than +0.45 observed 7 times each).
“GENE Y”: KIF13A (correlation score, R, greater than |-0.451 observed 13 times). GNA11 (correlation score, R, greater than |-0.45|observed 12 times). DST (correlation score, R, greater than |-0.45|observed 11 times). ACTN1, VAMP3 (correlation score, R, greater than |-0.45|observed 10 times each). KIF1B (correlation score, R, greater than |-0.45|observed 9 times). PTPN21 (correlation score, R, greater than |-0.45|observed 9 times). FERMT2 (correlation score, R, greater than |-0.45|observed 9 times). ZYG11B (correlation score, R, greater than |-0.45|observed 9 times). BTG3 (correlation score, R, greater than |-0.45|observed 8 times). RDX, DAG1, GGCX, MPDZ (correlation score, R, greater than |-0.45|observed 7 times).

### Illustrating Example of PHASE I of the Aforementioned Method

Acquiring cancer gene expression biomarkers for susceptibility/resistance to Enzalutamide drug (NSC number: 766085). Used the method as above. Except that in this case, the smallest minimum correlation, at or above 0.3, which returned less than the maximum number of returnable results in the system (which is presently 2,000) was 0.4. With this, 1871 results returned.

“GENE Q”: MMP14 (correlation score, R, greater than +0.4 observed 12 times). GAP43 (correlation score, R, greater than +0.4 observed 11 times). HIPK2 (correlation score, R, greater than +0.4 observed 9 times). MYO10, RHEB, MDFIC, HNMT (correlation score, R, greater than +0.4 observed 8 times for each). DAD1 (correlation score, R, greater than +0.4 observed 7 times for each).

“GENE Y”: NUP210 (correlation score, R, greater than |-0.41 observed 14 times). HNRNPD (correlation score, R, greater than |-0.41 observed 11 times). PNISR (correlation score, R, greater than |-0.41 observed 8 times).

### Some Cancer Gene Expression Biomarkers, for Susceptibility/Resistance to Some Exemplary Cancer Drugs, Determined by PHASE I of the Aforementioned Method

After each drug name, in brackets, is the selected threshold value of x, and after each gene name, in brackets, is the number of observations of correlation (n) above that threshold value of x, wherein in most cases y was set at 7 (but in occasional, isolated cases decreased to 6 or 5 when more biomarkers were desired for a drug): Enasidenib (0.35): “GENE Q”: SLC1A4 (15), SCD (7); “GENE Y”: PRKAA2 (11), IGFBP7 (10), MAP1B (8), WARS2 (7), PTPRB (7), JAG1 (7), TWSG1 (7). Ivosidenib (0.3): “GENE Q”: BGN (8), SFRP2 (7), ZHX3 (7). Vorasidenib (0.55): “GENE Q”: ENAH (15), FLNA (15), MAP1B (14), PPP2R3A (14), CFL2 (10), KIF13A (10), RBMS3 (9), CYR61 (9), NFIB (8), PEA15 (8), THBS1 (8), BACE1 (7), PFN2 (7), CAV1 (7), TRPC1 (7), CNN3 (7), PALLD (7); “GENE Y”: NUP210 (9), RSL24D1 (7), TGDS (7), SVIP (7). Fluorouracil (0.4): “GENE Q”: NDUFB10 (5), RNPS1 (5), NOLC1 (5); “GENE Y”: MACF1 (17), BACE1 (8), UTRN (7), H2AFV (7). Bafetinib (0.4): “GENE Q”: RAB27A (17), GYPA (14), GYPB (13), RHAG (12), HBA1 (12), PRKCB (11), HIC2 (11), MAPK1 (10), PPIL2 (10), PKLR (9), THEM4 (9), RFESD (8), UROD (7), GFI1B (7); “GENE Y”: SMAD1 (12), ACTB (12), MYOF (12), KTN1 (11), ZFP90 (11), PARD3 (11), ANXA2 (9), RBMS2 (9), TPM1 (9), AJUBA (9), ALCAM (8), GADD45B (8), PAWR (8), SPIN1 (8), RSPRY1 (7), NFIB (7), SPTAN1 (7), RRAS2 (7), ACTG1 (7). Bortezomib (0.4): “GENE Y”: CAPS (11), GPM6A (11), ERBB2 (11), CHD9 (11), CLOCK (10), PTPRB (10), MFAP3L (8), SOX2 (8), WARS2 (8), SEC22B (8), ARHGEF4 (7), GFAP (7), RUNX2 (7), AGPS (7), EPHA3 (6), AMPD1 (5). Cabozantinib (0.35): “GENE Q”: CALU (19), MMP14 (13), FKBP7 (10), PXDN (10), CALD1 (10), CKAP4 (8), THBS2 (8), FAM114A1 (7), CREB3L1 (7), TGOLN2 (7), TBC1D8B (7), NAV1 (7); “GENE Y”: EZR (11), JAG2 (8), PM20D2 (7), RAB4A (7), EIF3M (7), RNGTT (6). Carfilzomib (0.35): “GENE Q”: IK (7), FAM122B (7), BUB3 (7), FBXO45 (7); “GENE Y”: SNRPN (29), ABCB1 (14), NR2F2 (14), PLCB4 (11), TRAMI (10), RAB3B (8), HCFC1R1 (7). Cisplatin (0.4): “GENE Q”: PTPRC (14), IKZF1 (12), KDMSA (9), SLA (9), ANP32E (8), CEP350 (8), CHD1L (8), RAB33A (7), WAS (7), ACAP1 (6), ARHGAP30 (6); “GENE Y”: KLF3 (11), EHF (11), AGAP1 (10), ITGB4 (10), EPB41L4B (9), MYO1G (7), CTDSPL (6), DLG3 (6). Cobimetinib (0.45): “GENE Q”: PARVB (14), ETV5 (13), DUSP4 (12), MITF (11), RASSF3 (11), ROPN1 (10), SPRED1 (9), DUSP6 (8), GPR56 (8), INPPSF (8), PRKCD (8), SOX10 (8), THEM4 (8), PLEKHB2 (7), SORD (7), TYRO3 (7); “GENE Y”: NUCKS1 (12), ZFP90 (7). Crizotinib (0.4): “GENE Q”: PTPRC (11), KIR2DL1 (11), PDE4DIP (10), ALK (9), PRPF40A (9), KIR2DL4 (9), HLA-DRB1 (9), MXD1 (9), PSD4 (8), SERPINB3 (8), SERPINB4 (8), SOCS1 (8), TLE3 (7), TMEM260 (7); “GENE Y”: RBMS2 (16), LAPTM4B (11), KIF13A (10), RRAS2 (8), ACTN1 (7), CFL2 (7), DHCR24 (7), RAI14 (7), SORBS3 (7). Dabrafenib (0.45): “GENE Q”: SOX6 (16), PARVB (15), GAS7 (13), SNCA (13), SASH1 (12), MITF (11), MLANA (10), SGCD (10), RASSF3 (10), ROPN1 (10), DCT (9), FAM210A (9), ZFP106 (9), INPP5F (9), STX7 (9), BACE2 (8), RXRG (8), SOX10 (8), CAPN3 (8), DUSP4 (8), GSN (8), THEM4 (7), ACSL3 (7), EDNRB (7), TYR (7), ITGA9 (7), VAT1 (7), LZTS1 (7), QPCT (7), SPRED1 (7), ACP5 (6); “GENE Y”: GEMIN2 (12), SLC38A1 (11), HNRNPC (7). Dasatinib (0.45): “GENE Q”: VCAN (16), CFLAR (14), BIN1 (12), PARD3 (12), RBPMS (12), TFPI (11), LPP (10), CAV2 (10), PTRF (10), TLDC1 (10), RTN4 (9), PRKCA (9), ROR1 (9), TRIM8 (9), TPM1 (8), EXT1 (8), TRPC1 (8), MICAL2 (8), NR2F2 (8), NRIP1 (8), NRP1 (7), TWSG1 (7), AHNAK (7), AJUBA (7), AXL (7), EGFR (7), RRAS2 (7); “GENE Y”: IDH3A (9), WDR61 (8), GK (8), SLC25A11 (8), CBX4 (7), SMCR7L (7). Daunorubicin (0.35): “GENE Q”: PTPRC (14), IKZF1 (13), ZNRF1 (11), ACTG1 (9), PTPN22 (8), SLA (8), ARHGAP19 (7), PTPRN2 (7), MPHOSPH9 (7), MYO1G (7), NSL1 (7); “GENE Y”: ABCB1 (14), NR2F2 (13), PTK2 (9), ACTN1 (8), ITCH (7), LITAF (7), RBMS2 (7), TSTA3 (7). Docetaxel (0.4): “GENE Q”: IK (7); “GENE Y”: FAM19A5 (13), APOE (12), PLXNC1 (11), ZMAT3 (10), CDK2 (10), ATP6VOC (9), IVNS1ABP (9), BCAN (8), ARHGEF40 (7), MDM2 (7), NDE1 (7), NPR1 (7), PRTG (7), USP53 (7). Doxorubicin (0.35): “GENE Q”: ZNRF1 (9), AR (8); “GENE Y”: ABCB1 (14), PTK2 (11), NR2F2 (10), PTPN4 (8), TSTA3 (7), RBMS2 (7), HOOK1 (7), ITCH (7). Epirubicin (0.4): “GENE Q”: ZNRF1 (7); “GENE Y”: ABCB1 (14), PTK2 (11), NR2F2 (10), RBMS2 (7). Erlotinib (0.4): “GENE Q”: PAX8 (21), RBMS2 (16), PARD3 (12), RBPMS (11), CAV2 (11), RAB3B (11), LAPTM4B (10), LEPROT (9), FAM45A (9), GLRB (9), TLDC1 (9), PPP1R9A (8), MPZL1 (8), CDR2L (8), CNIH4 (8), RASAL2 (8), RBKS (8), PDZK1IP1 (7), CD24 (7), EGFR (7), ALDH3B1 (7), DCAF6 (7), RRAS2 (7), VCAN (7); “GENE Y”: TMEM161B (10), DKC1 (9), RBM10 (8), THOC2 (8), GTF3A (7), ACAA1 (6). Etoposide (0.4): “GENE Q”: TRPS1 (12), GATA3 (11), MUC1 (11), ESR1 (10), PDCD4 (10), PRLR (10), SPDEF (9), MED13L (8), SCAMPI (8), PTGER4 (7), CRNDE (7), FOXA1 (7); “GENE Y”: ANXA2 (10). Gefitinib (0.35): “GENE Q”: GLS (14), PAX8 (11), RBPMS (11), SPP1 (10), ANXA4 (9), MPZL1 (9), PPP1R9A (8), PDZK1IP1 (7), PON2 (7); “GENE Y”: BRD8 (8), ARIH2 (8). Imatinib (0.35): “GENE Q”: GYPB (15), GYPA (14), HIC2 (13), GATA2 (13), PRKCB (13), HBA1 (12), RHAG (12), IGF1 (9), PPIL2 (9), PKLR (9), UROD (9), RFESD (8), CRKL (8), NUP214 (7), GFI1B (7), ACSM3 (7), CISH (7), CPED1 (7), RHD (7); “GENE Y”: TLE1 (9), TWF1 (8), FAM45A (7). Ixabepilone (0.35): “GENE Q”: HAPLN1 (13), DTNA (7), ADO (7); “GENE Y”: RASAL2 (13), WWC1 (9), ATXN10 (8), TAOK1 (7), ZAK (7). Lapatinib (0.35): “GENE Q”: PAX8 (11), PDZK1IP1 (7), PPP1R9A (7); “GENE Y”: CUL4B (10). Lenvatinib (0.4): “GENE Q”: GBP1 (11), TPM1 (10), PXDN (10), TRPC1 (9), TMCC1 (8), BMP1 (8), CCDC80 (8), CYR61 (8), MICAL2 (8), PEA15 (8), PLOD2 (8), PRSS23 (8); “GENE Y”: AMD1 (11). Mitomycin (0.45): “GENE Q”: CD44 (15), MAP1B (14), TAOK1 (11), CAMSAP2 (10), SMARCA1 (10), HFE (9), OSMR (8), CKAP4 (8), LARP6 (8), CAV1 (7), PFN2 (7), RTN4 (7); “GENE Y”: DCAF11 (7), NUP210 (7), SMARCC1 (7). Nilotinib (0.3): “GENE Q”: PRKCB (18), GYPB (15), GYPA (14), HIC2 (13), RHAG (13), HBA1 (12), PPIL2 (11), IGF1 (9), MAPK1 (8), PKLR (8), RFESD (8), GFI1B (7), NUP214 (7); “GENE Y”: TRIO (11), NMT2 (10), CAMSAP2 (9), APBB2 (9), AJUBA (8), ITGA3 (8), SMARCA1 (8), ANXA2 (7), PFN2 (7), PRKAG2 (7), VEZF1 (7). Olaparib (0.35): “GENE Q”: ELL2 (14), IGFBP5 (14), D102 (13), GLIPR1 (11), FGF5 (10), HGF (10), PDE5A (10), NAMPT (9), CLU (8), LTBP1 (8), VAMP4 (8), WNT5A (8), PCDH18 (7), BCL3 (7), CDK14 (7), HIPK2 (7), S1PR3 (7), YIPF5 (7); “GENE Y”: CTDSPL (9). Oxaliplatin (0.45): “GENE Q”: RPL4 (9), DDX18 (9), HNRNPA1 (8), USP7 (8), HNRNPC (7); “GENE Y”: HOMER3 (8), GNA11 (7), LPP (7), NACA (7), RSL24D1 (7). Paclitaxel (0.35): “GENE Q”: FBXO45 (12), ARPC5L (8), BUB3 (7); “GENE Y”: PLCB4 (12), ABCB1 (12), MDM2 (11), NR2F2 (11), RAB3B (11), KIAA1033 (10), RASAL2 (9), APOE (8), TRPM1 (8), UGCG (8), FAS (7), LEPROT (7), PDZK1IP1 (7), TRAM1 (7), ZMAT3 (7). Pazopanib (0.35): “GENE Q”: GBP1 (8), HLA-E (7), OPTN (7), SP 110 (7), TRIM38 (7); “GENE Y”: SESN3 (15), CAPS (11), ETNK1 (11), SOX2 (9), DACH1 (8), GCLC (8), GSTTI1 (7), SRSF8 (7). Pemetrexed (0.35): “GENE Q”: THUMPD1 (8), METAP2 (8), ESD (8), CDT1 (7), NAP1L1 (7); “GENE Y”: MAP3K13 (8), SOX13 (7). Rapamycin (0.4): “GENE Q”: PLOD2 (9), FERMT2 (8), PDLIM7 (8), PKIA (8), RBMS1 (8); “GENE Y”: PTCD3 (10), LRPPRC (9), AKAP1 (8), PRPS2 (7), SLC35D2 (7), SORD (7). Ruxolitinib (0.4): “GENE Q”: TPM1 (15), VCAN (15), OPTN (12), CCDC80 (11), EHD2 (10), LTBP2 (9), CYP1B1 (8), PAPSS2 (8), PLOD2 (8), NNMT (7), CDH2 (7), FBN1 (7), GALNT1 (7), HRH1 (7), INHBA (7), JAKI (7), PAM (7). Selumetinib (0.4): “GENE Q”: PARVB (15), ETV5 (12), DUSP4 (12), MITF (11), RASSF3 (10), ROPN1 (10), STX7 (10), GSN (9), SPRED1 (9), RXRG (8), SGCD (8), STK10 (8), SNCA (8), PRKCD (8), ZFP106 (8), SOX10 (8), CREBL2 (8), MLANA (8), GPR56 (8), LYST (8), CAPN3 (7), FAM210A (7), SORD (7), THEM4 (7), TMTC2 (7), DUSP6 (6), ERBB3 (6); “GENE Y”: NUCKS1 (17), KTN1 (10), TBL1X (10), GADD45B (9), ZFP90 (9), BAD (8), MALAT1 (8), SPIN1 (8), GEMIN2 (7), MYL6 (7), VEZT (7). Sonidegib (0.4): “GENE Q”: JMJD6 (6), LAPTM4B (6); “GENE Y”: STK17B (7), N4BP2L1 (6). Sorafenib (0.4): “GENE Y”: PARD3 (10), AK4 (9), PAX8 (8), CD24 (7), LAMC2 (7), PPP1R9A (7), TSPAN2 (7). Sunitinib (0.4): “GENE Q”: HNF4A (8), ADD3 (7); “GENE Y”: IL6ST (11), EML1 (8), RDX (8). Tamoxifen (0.45): “GENE Q”: ANAPC5 (11), GTF3A (11); “GENE Y”: RBMS2 (12), ANXA2 (12), NNMT (11), RRAS2 (9), CRIM1 (8), CLIP4 (8), ITSN1 (8), DST (7), WWC1 (7). Temsirolimus (0.35): “GENE Q”: FAS (16), MR1 (9), CUX1 (7), TOR1A (7); “GENE Y”: TCF7L2 (18), EML4 (14), CTBP2 (12), ITGA6 (11), ADD3 (10), ASXL2 (8), GPD2 (8), AGAP1 (7), CHKA (7), ITGB4 (7), SLC16A5 (7), SORD (7). Trametinib (0.45): “GENE Q”: DUSP4 (11), RASSF3 (11), SPRED1 (8); “GENE Y”: NUCKS1 (9). Vemurafenib (0.45): “GENE Q”: SGCD (18), RAB27A (14), SNCA (14), MLANA (12), PARVB (12), GAS7 (11), DUSP4 (11), MITF (11), SASH1 (11), RASSF3 (10), ROPN1 (10), TRIB2 (10), AP1S2 (9), CSPG4 (9), LZTS1 (9), ETV5 (9), ZFP106 (9), SOX10 (8), BACE2 (8), RXRG (8), GSN (8), ITGA9 (8), ASAH1 (7), FAM210A (7), INPP5F (7), MREG (7), TYR (7); “GENE Y”: SLC38A1 (8), GADD45B (7). Vinblastine (0.35): “GENE Q”: HNRPLL (8); “GENE Y”: LEPROT (14), ABCB1 (12), PLCB4 (12), FAM49A (11), RAB3B (11), UGCG (11), NR2F2 (10), PARD3 (9), PAX8 (8), CLDN1 (7), CLEC4E (7), DENND3 (7), EPS8L2 (7), PDZK1IP1 (7), RBMS2 (7), TMEM178B (7). Vincristine (0.35): “GENE Q”: THOC2 (7); “GENE Y”: SCAND1 (15), CXCL5 (10), TGM2 (9), PON2 (9), NR2F2 (9), FGB (9), TMEM106B (9), SCEL (8), RBMS2 (7), FGA (7), FGG (7), CYP4F3 (7), DIRAS3 (7), AKAP13 (7), CHST9 (7), TLDC1 (7). Anastrazole (0.3): “GENE Q”: SSX3 (16), PPFIA1 (14), ST13 (11), FCRLA (10), HLA-DRB1 (9), RANGAP1 (9), NRXN1 (8), SMDT1 (8), SSX2 (8), XPNPEP3 (7), SSX1 (7), DESI1 (7), FGF13 (7), GBA3 (7), MYOZ2 (7), NHP2L1 (7), POLR3H (7), SSX4B (7); “GENE Y”: UBXN4 (6). Bendamustine (0.35): “GENE Q”: CDK6 (21), IKZF1 (16), FYB (15), TFDP2 (15), CD84 (11), GIMAP5 (11), NKAIN4 (11), ITGA4 (11), CASP2 (11), BCL11B (9), LDLRAD4 (9), GIMAP6 (8), CD6 (8), CXCR4 (8), ERG (8), LAT (8), CFTR (7), CRABP1 (7), IL4 (7), PDE7A (7); “GENE Y”: ANXA2 (11), AGAP1 (10), CTBP2 (10), CTNNA1 (8), PTK2 (7). Bleomycin (0.4): “GENE Q”: RORA (13), SOCS3 (13), PDE4DIP (11), GAS1 (10), ALK (10), HIPK2 (10), HLA-DRB1 (9), KIR2DL4 (9), SOCS1 (9), SERPINA1 (8), SERPINB3 (8), SERPINB4 (8), TRPC1 (8), CD274 (7), HLA-DQB1 (7), HTRA3 (7), IRF5 (7), TMEM260 (7); “GENE Y”: SORL1 (9), CHKA (8), RDH13 (7). Carmustine (0.35): “GENE Q”: GYPB (15), GYPA (14), HIC2 (13), PRKCB (13), HBA1 (12), RHAG (12), UROD (11), IGF1 (10), PPIL2 (10), RFESD (8), RHD (8), NUP214 (7), ACSM3 (7), CPED1 (7), GFI1B (7), PKLR (7); “GENE Y”: AKAP13 (16), PCDHA (11), ALDH3A2 (9), SPTAN1 (8), DOCKS (8), APP (7). Cyclophosphamide (0.35): “GENE Q”: PRLR (29), PTGER3 (23), TRPS1 (16), MUC1 (11), MED13L (9), OLFM1 (9), RABEP1 (9), GATA3-AS1 (8), MGP (8), PGR (8), ABCC6 (7), ANXA9 (7), DNALI1 (7), ELF5 (7). Dacarbazine (0.4): “GENE Q”: IKZF1 (22), PTPRC (13), LCP2 (12), CDK6 (11), CHD4 (10), PTPN22 (10), LAPTM5 (9), LRMP (8), WAS (8), MAP3K7 (8), SLA (8), ETV6 (7), ITGA4 (7), RNF138 (7); “GENE Y”: CTBP2 (13), GNA11 (9), ARHGAP35 (8), TACC2 (7). Estramustine (0.4): “GENE Q”: ELL2 (8), GAS1 (7), SURF4 (7). Gemcitabine (0.35): “GENE Y”: DLG3 (14), MYH14 (13), EHF (10), CXADR (9), TRAK1 (9), CLDN4 (8), TMEM45B (8), KLF4 (7), OVOL2 (7), SHROOM3 (7). Idarubicin (0.35): “GENE Q”: NAP1L1 (19), EIF1 (9), NSL1 (8), MPHOSPH9 (7); “GENE Y”: ABCB1 (13), CDKN2A (9), CCDC8 (8), CHKA (8), RIMS2 (8), SNRPN (8), AMOTL1 (7), ITCH (7), TSTA3 (7). Ifosfamide (0.4): “GENE Q”: BCL11A (17), TFDP2 (13), ITGA4 (9), LEF1 (9), BCL11B (8), LRMP (8), SH2D1A (8), CHRNA3 (7), NFATC3 (7), FYB (7), GIMAP6 (7), OGN (7), LDLRAD4 (7), SLIT1 (7), TOX (7); “GENE Y”: LPAR1 (7). Lomustine (0.35): “GENE 0”: RORA (17), PDE4DIP (16), KIR2DL4 (13), SOCS3 (12), ALK (10), GAS1 (10), KIR2DL1 (10), TMEM260 (10), HLA-DQB1 (9), HLA-DRB1 (9), SERPINA1 (9), SOCS1 (9), SERPINB3 (8), SERPINB4 (8), TSHZ2 (8), IRF5 (8), KIAA0226L (8), RORC (8), ACPP (7), HTRA3 (7), IL2RA (7), JAK3 (7); “GENE Y”: GNA11 (9). Melphalan (0.4): “GENE Q”: IKZF1 (22), PTPRC (16), N4BP2L1 (14), SLA (11), NAP1L1 (10), ITGA4 (9), WAS (9), ZNRF1 (9), SLC16A7 (9), ACAP1 (7), PTPN22 (7); “GENE Y”: CHKA (9), AGAP1 (8), BAIAP2L1 (8), MYO1G (7). Methotrexate (0.4): “GENE Q”: HNRNPC (16), PTBP1 (10), HNRNPD (8), RFC5 (8), SET (8), THUMPD1 (8), SART3 (7), KARS (7), METAP2 (7), MST4 (7), NUDT21 (7); “GENE Y”: NOTCH2 (13), CAP1 (7), DUSP10 (9). Mitoxantrone (0.4): “GENE Q”: NAP1L1 (22), NUCKS1 (14), ZNRF1 (9), ITGA4 (7), MPHOSPH9 (7); “GENE Y”: EXOSC4 (8), CHKA (8), TGFBR3 (8). Mustine (0.4): “GENE Q”: NAP1L1 (17), PTPRC (13), HNRNPC (10), NSL1 (10), MRPL9 (9), PSD4 (8), SLA (8), DDX23 (7), MYO1G (7); “GENE Y”: CALD1 (17), GNA11 (14), ACTN1 (10), RRBP1 (9), RBFOX2 (7), TRIOBP (7), TIMP3 (7). Procarbazine (0.3): “GENE Q”: CNTN1 (9), SLMO2 (9), WWTR1 (8), PTGDS (7). Raloxifene (0.4): “GENE Q”: NCOA3 (12), TRIM33 (12), RAD51C (10), ESR1 (9), RPS6KB1 (9), SPDEF (9), DHPS (8), HIPK1 (8), GATA3 (8), BRIP1 (7), BCL2 (7), DTWD1 (7); “GENE Y”: ANXA2 (15), RBMS2 (8). Vinorelbine (0.35): “GENE Q”: FBXO45 (8), ARPC5L (7), SERBP1 (7); “GENE Y”: NR2F2 (13), CCND1 (9), CTAG1A (9), PON2 (9), FGA (8), TGM2 (8), RAB3B (8), RASAL2 (8), FGB (8), CHST9 (7), CXCL5 (7), HCFC1R1 (7), LEPROT (7), ME3 (7), PDZK1IP1 (7). Abiraterone acetate (0.35): “GENE Y”: TRO (7), GBA3 (7), HEXIM1 (7). Capecitabine (0.3): “GENE Q”: SSX3 (16), PPFIA1 (11), ST13 (11), NRXN1 (9), RANGAP1 (9), FCRLA (9), HLA-DRB1 (9), NHP2L1 (8), SMDT1 (8), SSX2 (8), DESI1 (7), FGF13 (7), GBA3 (7), MYOZ2 (7), SSX1 (7), SSX4B (7). Darolutamide (0.4): “GENE Q”: LST1 (17), LILRA2 (12), AIF1 (10), ARHGAP9 (10), LCP2 (10), MPO (8), ARHGAP25 (7), LMO2 (7), RASGRP2 (7), WAS (7); “GENE Y”: CTNNA1 (21), ANXA2 (15), CTBP2 (13), IKZF1 (13), CMTM4 (9), TWF1 (9), BIN1 (8), CRK (8), GNA11 (8), DOCK1 (7), NCKAP1 (7), PFN2 (7), TSPAN6 (7). Venetoclax (0.35): “GENE Q”: LST1 (17), LILRA2 (15), ZADH2 (11), STK10 (10), MPO (9), IL17RA (8), KIAA0930 (8), LYST (8), NCF4 (8), PLEK (8), ME2 (8), TARP (8), AIF1 (7), ARHGAP25 (7), FRY (7), PRKCD (7), RASGRP2 (7), TFEC (7); “GENE Y”: ANXA2 (11), CRK (8), EZR (8), AMFR (7). Vindesine sulfate (0.4): “GENE Y”: SEMA6A (16), CEACAM1 (13), RHOQ (12), APOE (11), CDK2 (9), TRPM1 (9), DUSP10 (9), MBP (9), PLXNC1 (9), RGS12 (9), LDB3 (8), MMP8 (8), MCAM (8), TNS1 (8), ATP6VOC (7), PRTG (7), ITGA7 (7), MLANA (7), STX7 (7). 6-Mercaptopurine (0.4): “GENE Q”: CHD4 (9), LYAR (8), RCL1 (8), ZNRD1 (8), GART (7), HNRNPD (7), MRPL42 (7), RPL3 (7), U2AF1 (7), UBE2N (7); “GENE Y”: ATP6VOE1 (11), CHST3 (9), CLIP4 (7), EPHA4 (7), LHFP (7), ZBTB4 (7). Actinomycin D (0.4): “GENE Q”: FBXO45 (10); “GENE Y”: RBMS2 (19), ABCB1 (14), NR2F2 (13), AJUBA (10), WWC1 (10), LEPROT (10), NNMT (9), TRAM1 (9), TGM2 (9), BIN1 (8), MPP5 (8), KTN1 (8), PROSER2 (8), NXA2 (7), ITM2B (7). Azacitidine (0.35): “GENE Q”: CACYBP (9); “GENE Y”: GTF2I (10), PSEN2 (9), HS2ST1 (7), CD99 (7), FAM65B (7), PDE3A (7). Belinostat (0.4): “GENE Y”: NRXN3 (7). Busulfan (0.35): “GENE Q”: IKZF1 (21), PTPRC (16), KIR2DL1 (12), KIR2DL4 (11), SLA (11), SOCS3 (10), ALK (9), WAS (9), N4BP2L1 (8), RORA (8), TMEM260 (8), SERPINB3 (8), SERPINB4 (8), ACAP1 (7), CDK6 (7), MYO1G (7), RAB33A (7); “GENE Y”: CTDSPL (10), AGAP1 (9), KIF13A (8), KLF3 (8), YWHAZ (8). Chlorambucil (0.45): “GENE Q”: IKZF1 (22), PTPRC (16), N4BP2L1 (13), LCP2 (11), SLA (11), ITGA4 (10), ARHGAP9 (9), FNBP1 (8), PTPN22 (8), WAS (8), ACAP1 (7), AIF1 (7), CD84 (7), FMNL1 (7), MYO1G (7), ANP32E (6); “GENE Y”: AGAP1 (11), CTDSPL (10), DDR1 (10), KLF3 (10), DOCK6 (8), LMNA (7), ATP6V1A (7), BAIAP2 (7), BAIAP2L1 (7), TMBIM1 (7). Cladribine (0.35): “GENE Q”: JRK (8), SNRPN (8), NUDT21 (7), RNGTT (7); “GENE Y”: TTC3 (10), AHCYL1 (7). Clofarabine (0.35): “GENE Q”: PDS5A (9), PRPF8 (8); “GENE Y”: SPON1 (15), FGF18 (10), ITGB8 (9), DR1 (8), ERBB4 (8), FZD8 (7), SHROOM3 (7). Cytarabine (0.4): “GENE Q”: ESD (11), MPHOSPH9 (10), NAP1L1 (9), ARHGAP19 (8), CAND1 (8), KDM5A (8), MST4 (8), CCND3 (7), CTCF (7), EP400 (7), HNRNPC (7); “GENE Y”: PHACTR2 (8), PTPN21 (8). Decitabine (0.35): “GENE Q”: GK (11), PTPRC (9), RABEP1 (9), TBRG1 (8), BUB3 (7), PCM1 (7); “GENE Y”: CTNNA1 (13), BMPR2 (10), LAPTM4B (8), RAI14 (8), RRAS2 (8), CDKN1C (7), ITM2B (7), RAB1A (7). Eribulin mesilate (0.4): “GENE Q”: ATP11B (7); “GENE Y”: PRLR (24), ABCB1 (14), UGCG (14), NR2F2 (13), PLCB4 (12), PTGER3 (10), JAKI (9), RAB3B (9), LEPROT (9), ABCC6 (8), CLDN1 (8), DCAF6 (7), DNALI1 (7), MATN3 (7). Exemestane (0.35): “GENE Q”: CAPN3 (13), FCRLA (10), SSX3 (9), EDNRB (8), ST3GAL6 (8), TRPM1 (8), POLR2F (7), SLC11A2 (7). Floxuridine (0.35): “GENE Q”: NAP1L1 (11), MED13 (10), NXPE3 (9), FGF2 (8), TAOK1 (8); “GENE Y”: CSF2RA (10), CHKA (9), MPZL2 (8), SCEL (7). Fludarabine (0.35): “GENE Q”: IKZF1 (18), TFDP2 (14), BCL11A (12), CHRNA3 (9), PTPRC (9), BCL11B (8), SH2D1A (8), GIMAP6 (7), ITGA4 (7), N4BP2L1 (7), SLIT1 (7), ZNF22 (7); “GENE Y”: AGAP1 (11), ITGB5 (8), CEP170B (7), GGA2 (7). Fulvestrant (0.4): “GENE Q”: NCOA3 (15), ESR1 (13), GATA3 (11), PDCD4 (11), APPBP2 (10), HBA1 (10), RAD51C (10), RPS6KB1 (10), CYB561 (9), SCAMPI (9), SPDEF (9), TUBD1 (9), BCL2 (8), PPM1D (8), BRIP1 (7), DTWD1 (7), GSE1 (7), HEATR6 (7), HIPK1 (7), SIAH2 (7), TBC1D30 (7); “GENE Y”: BIN1 (8), ANXA2 (7), SPTBN1 (7). Hexamethylmelamine (0.3): “GENE Q”: ABCB1 (14), DOK4 (10), SNRPN (8), CDX2 (7), KIF26A (7); “GENE Y”: RTFDC1 (7). Irinotecan (0.35): “GENE Q”: NAP1L1 (16), PRPS1 (9), BCAT1 (7), DNAJC8 (7), MST4 (7), YWHAE (7); “GENE Y”: CHKA (9), BCR (8), SHROOM3 (8), BAIAP2L1 (7), LMOD1 (7). Irofulven (0.4): “GENE Q”: ANXA2 (10), CTBP2 (10), BMPR1A (7), GNA11 (7); “GENE Y”: IGKC (21), IGLC1 (19), TCF4 (18), MAF (13), FAM26F (11), CCR1 (11), CCND2 (9), FAM46C (9), IGHG1 (9), CD28 (9), CD8B (8), EPB41 (8), TNFRSF18 (7), IGLJ3 (7), IQGAP2 (7), MZB1 (7), PIM2 (7). Ixazomib citrate (0.35): “GENE Q”: OPA1 (11), HPS1 (8), SLC25A36 (8), CREBRF (7); “GENE Y”: SCAND1 (15), GCLC (11), PHF20 (10), SLC7A11 (9), SLITRK6 (9), CYP4F3 (8), SCEL (8), ALDH3A2 (7), CYP1B1 (7), ERGIC2 (7), OCIAD1 (7), PHKB (7), PTHLH (7), RBM39 (7), SLAIN2 (7). Letrozole (0.35): “GENE Q”: SSX3 (15), ASXL1 (8), SBSPON (8), SSX2 (8), GBA3 (7), RANGAP1 (7). Mitomycin C (0.35): “GENE Q”: CHD9 (7), JAM3 (7), KYNU (7), MPHOSPH9 (7), NUFIP2 (7), NXPE3 (7); “GENE Y”: KLF3 (9), SHROOM3 (8), TES (8), SLC17A5 (7). Mitotane (0.35): “GENE Q”: PRLR (28), PTGER3 (16), TRPS1 (16), LPAR1 (13), MUC1 (11), STC2 (9), BNIP3L (7), KCTD6 (7), KDM4B (7), MGP (7), PGR (7), PTGER4 (7), RABEP1 (7), SLC24A3 (7). Nelarabine (0.3): “GENE Q”: SSX3 (16), ST13 (12), FCRLA (10), SMDT1 (9), CAPN3 (8), NRXN1 (8), PPFIA1 (8), RANGAP1 (8), SSX2 (8), FGF13 (7), GBA3 (7), MYOZ2 (7), NHP2L1 (7), PEXSL (7), PFKFB4 (7), POLR2F (7), SSX1 (7), SSX4B (7); “GENE Y”: HFE (8). Pentostatin (0.3): “GENE Q”: TRAM1 (10), GNG4 (7), VCAN (7). Pralatrexate (0.4): “GENE Y”: STC2 (9), ERBB3 (8), STK3 (7). Raltitrexed (0.4): “GENE Q”: NAP1L1 (11); “GENE Y”: CHKA (8). Temozolomide (0.35): “GENE Q”: PTPRC (15), IKZF1 (14), COL4A3 (9), KIAA1551 (9), SLA (8), PTGIS (7), PTPN22 (7), STATSB (7), TMOD1 (7). Teniposide (0.4): “GENE Q”: IKZF1 (20), NAP1L1 (20), MPHOSPH9 (12), PTPRC (11), ITGA4 (9), ST8SIA4 (9), SLA (9), PTPN22 (8), SSBP2 (8), FMNL1 (7), NSL1 (7), ZNRF1 (7); “GENE Y”: ABCB1 (13), PTK2 (10), ITCH (7), KLF3 (7), TSTA3 (7). Thioguanine (0.35): “GENE Q”: LARP4B (9), RAC2 (7); “GENE Y”: SESN3 (16), GPM6A (13), SEC22B (12), TKT (12), CAPS (11), TRIM2 (9), EPHA3 (8), FBXO32 (8), PTN (8), WARS2 (8), AGPS (7), GFAP (7), IGFBP7 (7), NEDD9 (7), PDGFRA (7). Topotecan (0.35): “GENE Q”: NAP1L1 (15), HDGFRP3 (8), MPHOSPH9 (7), STRADA (7), TAOK1 (7); “GENE Y”: BAIAP2L1 (9), CHKA (9), NPAS2 (9), TES (9), TGFBR3 (7). Toremifene (0.4): “GENE Q”: PSD4 (10), GTF3A (9), TLE3 (8), BCL2 (7), FOXP2 (7), RAD51C (7); “GENE Y”: LRP6 (8), ANXA2 (8), CORO1C (7). Uracil mustard (0.4): “GENE Q”: IKZF1 (22), NAP1L1 (16), PTPRC (16), N4BP2L1 (13), SLA (11), ARHGAP9 (10), ITGA4 (10), LST1 (10), AIF1 (8), PTPN22 (8), WAS (8), MPHOSPH9 (8), ACAP1 (7), CD84 (7), MYO1G (7); “GENE Y”: KLF3 (12), BAIAP2L1 (10), AGAP1 (10), GNA11 (9), CTDSPL (7), DOCK6 (7), TMBIM1 (7). Valrubicin (0.4): “GENE Q”: IKZF1 (20), NAP1L1 (19), PTPRC (14), HNRNPC (10), ITGA4 (9), CDK6 (8), MST4 (8), ST8SIA4 (8), MPHOSPH9 (8), FMNL1 (7), RPL23A (7), SLA (7); “GENE Y”: VAMP3 (9), CTDSPL (9), RALGPS2 (9), CHKA (7). Vorinostat (0.4): “GENE Q”: CHD4 (7), GDAP1 (7); “GENE Y”: PLS3 (7). Risperidone (0.3): “GENE Q”: EHF (6), SLC6A2 (5), EGFR (5), GM2A (5).

A method of identifying a subject with cancer eligible for treatment with a drug comprising testing a biological sample(s) from the subject, wherein the subject is eligible for treatment with the drug if analysis of the sample(s) indicates/ascertains that the subject's cancer's expression of one or more of this drug's “GENE Q” is above a threshold amount (e.g. a threshold amount as defined elsewhere herein; and/or is substantially similar to that of a cancer[s] observed/known/reported to be responsive/susceptible to this drug), and/or the subject's cancer's expression of one or more of this drug's “GENE Y” is below a threshold amount (e.g. a threshold amount as defined elsewhere herein; and/or is substantially similar to that of a cancer[s]observed/known/reported to be responsive/susceptible to this drug). Optionally wherein part or all of this method is performed by at least one “companion diagnostic”, wherein the metes and bounds of this term are well understood to those of the art. Optionally wherein the companion diagnostic comprises at least one microarray.

A method of treating cancer in a subject, comprising testing a biological sample(s) from the subject to ascertain if/whether the subject's cancer's expression of one or more of a drug's “GENE Q” is above a threshold amount (e.g. a threshold amount as defined elsewhere herein; and/or is substantially similar to that of a cancer[s] observed/known/reported to be responsive/susceptible to this drug), and/or
the subject's cancer's expression of one or more of a drug's “GENE Y” is below a threshold amount (e.g. a threshold amount as defined elsewhere herein; and/or is substantially similar to that of a cancer[s]observed/known/reported to be responsive/susceptible to this drug),
and, if affirmative, administering a therapeutically effective amount of this drug(s) to the subject, optionally atop of (in addition to) the standard of care treatment for this cancer type/diagnosis.

### Description Component in Claim Format:

[1] At least one microarray chip comprising/containing at least one nucleic acid probe(s) for (i.e. a single-stranded nucleotide sequence(s) that is complimentary for/hybridisable to, preferably with ≥85% sequence identity, to) mRNA and/or cDNA (and/or part(s) thereof, e.g. ≥15 consecutive nucleotides thereof) of one or more of “GENE Q” and/or “GENE Y” relating (e.g. as taught herein) to at least one or more drugs mentioned/specified herein, and/or relating to at least one or more approved/licensed (by one or more of the FDA, EMA, MHRA, PMDA, NMPA) cancer drugs.
[2] A microarray chip of Claim 1 which only reports upon a single “GENE Q” and/or a single “GENE Y” for each drug that it can predict cancer susceptibility/resistance to, preferably wherein, for each drug case, this is the “GENE Q” and/or “GENE Y” that has been observed to correlate to cancer susceptibility/resistance to this drug with the most observations thereof (above a threshold value of |R|, x), preferably wherein x is at least 0.3, optionally wherein if there is more than one “GENE Q” or “GENE Y” for a particular drug by this method, that selected for incorporation on the chip is all/some of these or singly that with greatest summed value of |R| across its observations.
[3] A microarray chip of Claim 2 which probes for (reports upon) the expression of a single “GENE Q” and/or “GENE Y” as disclosed for each drug herein, or some sub-fraction thereof, optionally wherein each “GENE Q” and/or “GENE Y” has multiple probes for it upon the chip.
[4] A microarray chip of Claim 1 which probes for (reports upon) the expression of one or more of MACF1, VPRBP, ERBB2, ANKH, STX12, GTF3A, KIF13A, MMP14, NUP210, SLC1A4, PRKAA2, BGN, ENAH, FLNA, NUP210, NDUFB10, RNPS1, NOLC1, MACF1, RAB27A, SMAD1, ACTB, MYOF, CAPS, GPM6A, ERBB2, CHD9, CALU, EZR, IK, FAM122B, BUB3, FBXO45, SNRPN, PTPRC, KLF3, EHF, PARVB, NUCKS1, PTPRC, KIR2DL1, RBMS2, SOX6, GEMIN2, VCAN, IDH3A, PTPRC, ABCB1, IK, FAM19A5, ZNRF1, ABCB1, ZNRF1, ABCB1, PAX8, TMEM161B, TRPS1, ANXA2, GLS, GYPB, TLE1, HAPLN1, RASAL2, PAX8, GBP1, AMD1, CD44, DCAF11, NUP210, SMARCC1, PRKCB, TRIO, ELL2, IGFBP5, CTDSPL, RPL4, DDX18, FBXO45, PLCB4, ABCB1, GBP1, SESN3, THUMPD1, METAP2, ESD, MAP3K13, PLOD2, PTCD3, TPM1, VCAN, PARVB, NUCKS1, JMJD6, LAPTM4B, STK17B, PARD3, HNF4A, IL6ST, ANAPC5, GTF3A, RBMS2, ANXA2, FAS, TCF7L2, DUSP4, RASSF3, NUCKS1, SGCD, SLC38A1, HNRPLL, LEPROT, THOC2, SCAND1, SSX3, UBXN4, CDK6, ANXA2, RORA, SOCS3, SORL1, GYPB, AKAP13, PRLR, IKZF1, CTBP2, ELL2, DLG3, NAP1L1, ABCB1, BCL11A, LPAR1, RORA, GNA11, IKZF1, CHKA, HNRNPC, NOTCH2, NAP1L1, EXOSC4, CHKA, TGFBR3, NAP1L1, CALD1, CNTN1, SLMO2, NCOA3, TRIM33, ANXA2, FBXO45, NR2F2, TRO, GBA3, HEXIM1, SSX3, LST1, CTNNA1, LST1, ANXA2, SEMA6A, CHD4, ATP6V0E1, FBXO45, RBMS2, CACYBP, GTF2I, NRXN3, IKZF1, CTDSPL, IKZF1, AGAP1, JRK, SNRPN, TTC3, PDS5A, SPON1, ESD, PHACTR2, PTPN21, GK, CTNNA1, ATP11B, PRLR, CAPN3, NAP1L1, CSF2RA, IKZF1, AGAP1, NCOA3, BINI, ABCB1, RTFDC1, NAP1L1, CHKA, ANXA2, CTBP2, IGKC, OPA1, SCAND1, SSX3, CHD9, JAM3, KYNU, MPHOSPH9, NUFIP2, NXPE3, KLF3, PRLR, SSX3, HFE, TRAM1, STC2, NAP1L1, CHKA, PTPRC, IKZF1, NAP1L1, ABCB1, LARP4B, SESN3, NAP1L1, BAIAP2L1, CHKA, NPAS2, TES, PSD4, LRP6, ANXA2, IKZF1, KLF3, IKZF1, VAMP3, CTDSPL, RALGPS2, CHD4, GDAP1, PLS3, EHF, SCAF11, ARL6IP5, optionally wherein those selected are the only genes whose expressions are probed for on the chip, optionally wherein less than 1,000, and/or less than 100, and/or less than 50, and/or less than 10 genes have their expressions probed for on the chip.
[5] A method of using the microarray chip of one or more of Claims 1 to 4 comprising contacting it with a (biological) sample(s) from the subject, measuring the hybridization, to predict/diagnose which of these drugs a subject's cancer is likely to be most susceptible/responsive to,
optionally wherein the subject is then administered with a therapeutically effective amount of said drug(s), optionally wherein if more than one such drug is administered, their anti-cancer activities in the subject are synergistic.
[6] A kit/companion diagnostic comprising at least one microarray chip of one or more of Claims 1 to 4.
[7] A microarray that only contains a probe(s) for one or more of the genes mentioned herein.
[8] The method of Claim 5 wherein this is repeated a number of times during the subject's cancer treatment, wherein if the most recommended drug(s) by this method changes, then a therapeutically effective amount of the newly recommended drug(s) is administered to the subject instead of, or in addition to, one or more of the previously most recommended drug(s) by this method.
[9] A written/audio/electronically encoded/computer medium report that recommends/suggests (explicitly, or implicitly by reported data) a cancer drug(s) treatment for a subject on the basis of one or more of the cancer gene expression biomarkers mentioned/taught herein, and optionally a method of generating such a report.
[10] The method of Claim 5 wherein the subject is administered with a therapeutically effective amount of one or more of Ibrutinib, Osimertinib, Palbociclib, Enzalutamide, Enasidenib, Ivosidenib, Vorasidenib, Fluorouracil, Bafetinib, Bortezomib, Cabozantinib, Carfilzomib, Cisplatin, Cobimetinib, Crizotinib, Dabrafenib, Dasatinib, Daunorubicin, Docetaxel, Doxorubicin, Epirubicin, Erlotinib, Etoposide, Gefitinib, Imatinib, Ixabepilone, Lapatinib, Lenvatinib, Mitomycin, Nilotinib, Olaparib, Oxaliplatin, Paclitaxel, Pazopanib, Pemetrexed, Rapamycin, Ruxolitinib, Selumetinib, Sonidegib, Sorafenib, Sunitinib, Tamoxifen, Temsirolimus, Trametinib, Vemurafenib, Vinblastine, Vincristine, Anastrazole, Bendamustine, Bleomycin, Carmustine, Cyclophosphamide, Dacarbazine, Estramustine, Gemcitabine, Idarubicin, Ifosfamide, Lomustine, Melphalan, Methotrexate, Mitoxantrone, Mustine, Procarbazine, Raloxifene, Vinorelbine, Abiraterone acetate, Capecitabine, Darolutamide, Venetoclax, Vindesine sulfate, 6 Mercaptopurine, Actinomycin D, Azacitidine, Belinostat, Busulfan, Chlorambucil, Cladribine, Clofarabine, Cytarabine, Decitabine, Eribulin mesilate, Exemestane, Floxuridine, Fludarabine, Fulvestrant, Hexamethylmelamine, Irinotecan, Irofulven, Ixazomib citrate, Letrozole, Mitomycin C, Mitotane, Nelarabine, Pentostatin, Pralatrexate, Raltitrexed, Temozolomide, Teniposide, Thioguanine, Topotecan, Toremifene, Uracil mustard, Valrubicin, Vorinostat, Risperidone, Almitrine (e.g. almitrine dimesylate).

### Almitrine & MYC

Almitrine dimesylate's anti-cancer activity correlates with MYC (c-MYC) gene (oncogene) expression (FIG. 1). Indeed, furthermore, in NCI COMPARE: correlation between MYC expression and cancer susceptibility to almitrine dimesylate: R>0.4 is observed 4 times and R>0.3 is observed 7 times, across 5 different gene expression data sets.

For many different kinds of cancer, high MYC expression/activity has been shown to correlate with drug(s) resistance and/or poor prognosis. Thence almitrine (and/or a pharmaceutically-acceptable salt, solvate, hydrate or prodrug thereof) is especially useful for the most dangerous/drug(s) resistant cancers. Especially because directly targeting MYC has thus far proven elusive in the clinic. Indeed, it is commonly termed “undruggable” because it is a dynamic disordered/unstructured protein lacking effective binding pockets on its surface [18]. 10058-F4 drug does disrupt MYC/Max heterodimerization but it isn't suitable for in vivo use because of fast degradation and low affinity for target [18]. And so indirect MYC targeting with the already clinical drug, almitrine (and/or a pharmaceutically-acceptable salt, solvate, hydrate or prodrug thereof), is a very useful clinical approach. Componentry to this disclosure is administration of almitrine and/or a pharmaceutically-acceptable salt, solvate, hydrate or prodrug thereof to treat/ameliorate/prevent/combat cancer in a subject, wherein this cancer exhibits notable MYC activity e.g. high level of MYC mRNA and/or high MYC protein amount (optionally assayed by an immuno technique e.g. immunoprecipitation i.e. high immunopositivity).

In blood plasma, circulating DNA fragments released by cells are known as cell-free DNA (cfDNA) and can be detected by quantitative real time PCR (q-PCR), wherein this method shows that, in blood plasma, breast cancer patients have greater MYC cfDNA than healthy controls, wherein this MYC amount increases with cancer progression, thence this “liquid biopsy” method [19] is one non-limiting method to determine a subject's cancer's susceptibility (correlating with MYC amount/activity) to almitrine (and/or a pharmaceutically-acceptable salt, solvate, hydrate or prodrug thereof) anti-cancer treatment.

Increased MYC expression/activity is a common feature of many cancers, including many haematological cancers/leukemias/lymphomas/myelomas (e.g. reviewed in [20-21]), often correlating with poor prognosis and/or drug(s) resistance: all well known by those of the art.

CML: In CML, greater MYC expression correlates with resistance to imatinib [22]. Wherein MYC knockdown sensitizes to imatinib [23]. Wherein there is “a positive correlation between MYC expression at diagnosis and poor response to imatinib”: “MYC mRNA levels are higher in nonresponders” [24]. MYC antagonizes imatinib/dasatinib-conferred differentiation of CML [25]. shRNA against MYC, or MYC inhibitor 10058-F4, exerts anti-CML activity, wherein 10058-F4 and imatinib exert synergistic anti-cancer activity [26]. From which, almitrine (and/or a pharmaceutically-acceptable salt, solvate, hydrate or prodrug thereof) and imatinib/dasatinib/nilotinib/radotinib/bosutinib/ponatinib are, by the teaching of this disclosure, anticipated to exert synergistic anti-CML activity. BCR-ABL fusion oncogene acts through MYC (possibly especially important in chronic to blast transition of CML) [27]. MYC expression is significantly higher in the accelerated/blastic phase than chronic phase of CML and it is possible that increased MYC expression drives the switch out of the chronic and into the more dangerous phases of CML [28]. LYMPHOMA: Increased MYC gene expression/activity is a characteristic feature of Burkitt's lymphoma, which can be because of chromosomal translocation e.g. causing MYC to be placed downstream of the highly active immunoglobulin (Ig) promoter region, leading to overexpression of MYC [29]. MYC translocations and/or increased MYC expression/activity is involved in many other lymphomas [30], typically conferring poor prognosis, including (without restriction) diffuse large B cell lymphoma (DLBCL), B-cell lymphoma unclassifiable (BCLU), Double hit/triple hit lymphomas, “high grade B-cell lymphoma with MYC and BCL2 and/or BCL6 translocations” (a WHO category), transformed or high-grade follicular lymphoma (FL), Mantle cell lymphoma (MCL), lymphomas with plasmablastic differentiation etc, wherein MYC inhibition can treat lymphoma [31]. AML: In AML, greater MYC expression correlates with lower overall survival (OS) (for AML with myelodysplasia related changes, AML-MRC) [32]. “Significant over-expression of MYC mRNA in AML patients compared to controls” and “higher MYC was consistently associated with poorer survival” and “higher MYC-immunopositivity conferring an inferior prognosis” [33]. MYC expression is elevated in AML patient samples and cell lines [34]. Increased MYC is seen in human Acute Promyelocytic Leukemia (APL) [35]. Overexpressing MYC can actually cause AML [36-38]. “Myc expression was positively correlated with drug resistance of leukemic cells, and could act as a significant clinical biomarker for AML prognosis” wherein Myc inhibition (MYC antisense RNA or 10058-F4 drug inhibitor) can overcome drug resistance [39]. Antisense RNA to MYC can induce myeloid differentiation [40]. shRNA knockdown of MYC, or administering a drug that INdirectly targets MYC, prolongs survival of mice with AML [41]. 10058-F4 drug inhibitor of MYC causes AML cells to differentiate and, at higher concentrations, induces their apoptosis [42]. MYC is absolutely required for the development of acute hematopoietic malignancies [43]. MYC activity is involved in the danger and drug resistance of FLT3-ITD fusion protein associated AML [44]. Wherein a significant enrichment of a MYC-related gene set in FLT3-ITD compared to FLT3-WT AML samples has been observed. 98% of FLT3-ITD mutated patients had high MYC-immunopositivity. MYC activity is upregulated by the activating mutations of FLT3 receptor tyrosine kinase, found in nearly one-third of all patients with AML. Plus by RUNX1-ETO, AML1-ETO, PML/RARα, and PLZF/RARα oncogenes in AML [36, 42]. An association is observed between higher MYC-immunopositivity and mutated NPM1 alone or dual NPM1 and CEBP mutations [33]. MYC gene, located at 8924, has been found to be one of the most commonly amplified regions in AML [45-46]. So, increased MYC activity is common to all these different types of AML. ALL: In ALL: T- or B-ALL (or both) occurs in zebrafish expressing transgenic murine/human MYC controlled by a zebrafish rag2 promoter, which is active in immature B and T lymphoblasts [47]. MYC suppression by small hairpin RNA prevents T-ALL initiation in a mouse model of T-ALL [488]. Overexpression of MYC in certain ALL cell lines, or primary patient samples, correlates with aberrantly prolonged MYC half-life due to abnormalities in the phosphorylation/dephosphorylation regulating MYC stabilization/degradation [49]. T-ALL is associated with elevated MYC expression driven by NOTCH1 wherein NOTCH1 inhibition decreases MYC mRNA levels and inhibits leukemic cell growth [50, 51, 52, 53, 54, 55]. MYELOMA: In myeloma, MYC is often overexpressed at the transcription [56-57] and/or translation level [58], wherein MYC overexpression is a marker for a more aggressive myeloma (such as plasma cell leukemia [59]) and poor prognosis [60]. Indeed MYC can be causal to pre-cancer becoming myeloma [61, 62, 63]. Wherein MYC inhibition/downregulation can kill myeloma cells [64, 65, 66, 67].

### Almitrine & Further Oncogenes

Cancer susceptibility to almitrine dimesylate correlates with USP36 gene expression (with microarray data, across many different data sets, but not with RNA-seq data: correlation (R) between RNA-seq composite gene expression (log 2[FPKM+1]) and mean[1-|5-]dose(10 μM)=0.09), wherein USP36 deubiquitinates and stabilizes MYC [68]. Cancer susceptibility to almitrine dimesylate correlates with (MYC driven) HNRNPA1 gene expression, wherein HNRNPA1 is a drive to alternative splicing of pyruvate kinase, favouring the embryonic pyruvate kinase isoform, PKM2, which is a metabolic switch into aerobic glycolysis/Warburg effect [69]. Thence cancer susceptibility to almitrine dimesylate correlates with PKM2 protein amount and high PKM2/PKM1 protein ratio. Cancer susceptibility to almitrine dimesylate correlates with (MYC driven [70]) PROM1 gene (coding for CD133 protein) expression, wherein CD133 is a cancer stem cell marker (expressed on cell surface and so very amenable to immuno assays to detect its amount), associated with chemoresistance and poor prognosis [71], e.g. with acute leukemia [72]. Cancer susceptibility to almitrine dimesylate correlates with SLC38A1, sodium-coupled neutral amino acid transporter 1, which transports glutamine, wherein high expression of SLC38A1 predicts poor prognosis with many cancers, including AML [73]. A disclosure embodiment(s) is to administer almitrine (and/or a pharmaceutically-acceptable salt, solvate, hydrate or prodrug thereof) to a subject that has, or that is suspected to have, or might have, a cancer(s) that favours glutaminolysis (low blood [glutamine] can be a sign of this), optionally wherein the subject adheres to a low glutamine and/or low protein diet.

That cancer susceptibility to almitrine dimesylate correlates with MYC, USP36, HNRNPA1, PROM1 (CD133 protein) and SLC38A1 expression suggests that the most dangerous, chemoresistant cancers are not least, but actually most, susceptible to the anti-cancer activity of almitrine (and/or a pharmaceutically-acceptable salt, solvate, hydrate or prodrug thereof), which makes almitrine an incredibly valuable addition to the anti-cancer drug armoury.

Cancer susceptibility to almitrine dimesylate positively correlates with expression of PTGS1, wherein high PTGS1 expression characterizes imatinib resistant, from susceptible, CML [74].

Cancer susceptibility to almitrine dimesylate correlates with the expression of the following genes, which have been reported to correlate with cancer danger in one or more cancer types: CAMKK2 [75], CBLL1 [76], DNAJC12 [77], FABP6 [78], ILF2 [79], NADSYN1 [80], NPTX2 [81], RNF144B [82], TMPRSS3 [83], ZNF277 [84], TPK1 [85], ANXA11 [86] and MLL2 (also known as KMT2D) [87], wherein MLL2 is especially important to MLL1-fusion protein driven AML [88].

### FIGURE LEGENDS

FIG. 1: Some cancer gene expression correlations to almitrine dimesylate susceptibility. Each data point relates to a cancer cell line. All gene expression data in this figure was sourced from the CellMiner database [3-4].

Square (▪) and round (●) data points relate to gene expression determined by Human Genome microarray chips made by the Affymetrix and Aligent companies respectively (for which the x-axis is mean log 2[intensity]). These two companies make microarray chips that differ in many design features e.g. probe length (25- vs. 60-mer). Triangular (▴) data points relate to gene expression determined by RNA-seq (for which the x-axis is log 2[FPKM+1], where FPKM is “Fragments Per Kilobase per Million reads”; “composite” data used {defined in main text}, rather than of any individual isoform). The concordance between the data from these two different methodologies is extremely high, which is validating.

Unadjusted, the Agilent data is much lower in value than the Affymetrix data. To make these closer in value, for ease of graphing and analysis: independently for each gene in question: the median expression of the gene across all the cancer cell lines was calculated for Affymetrix and Agilent data. The difference between these medians was then added to each Agilent gene expression data point for that gene, for every cancer cell line.

Unadjusted, the RNA-seq data is much lower in value than the Affymetrix microarray data. So, the same method was performed for the RNA-seq data. As aforementioned for Agilent data. To make all the data values closer to those of the Affymetrix microarray data. For ease of graphing and analysis.

Each Affymetrix data point is the mean of 4 different microarray data sets, sourced using 4 different Affymetrix Human Genome (HG) microarray chip sets: (1) GSE5720 data set sourced using the Affymetrix Human Genome U133A and U133B chips; (2) GSE32474 data set sourced using the Affymetrix Human Genome U133 Plus 2.0 chip; (3) GSE29682 data set sourced using the Affymetrix Human Exon 1.0 ST chip; (4) GSE5949 data set sourced using Affymetrix Human Genome U95A, U95B, U95C, U95D, U95E chips. The aforementioned GSE29682 data set has 4 probes per exon on average, and thence the number of probes for any given gene tends to scale with the number of exons it has, where the average gene has 10 exons and so there are, on average, 40 probes per gene on this chip. Focusing on just the GSE29682 data set alone, the chance that all these different probes for a single gene would correlate at sizable magnitude, all in the same direction, making the mean correlation sizeable in one direction, if there is no true correlation for that gene, is small.

The presented p-values in this figure are small. But they would probably be smaller still if raw, instead of mean, data was used. For each, this would increase n, R would likely still hold at sizable amplitude, decreasing p. Firstly, the y-axis is a mean of two datas, which could be used separately, doubling parameter n. Plus, the Agilent microarray data in this figure is a mean of underlying data in some cases, which could be utilized separately instead, increasing n. Furthermore, each Affymetrix microarray gene expression value for a cell line is a mean of multiple underlying mean data points, from 4 different independently generated (separated in time and space: generated at different times by different groups) microarray data sets, which could be used separately, increasing parameter n greatly. However, for expediency, means were utilized instead. With which there is still a sizeable value of n. Such that, given that each correlation R is high, and so p is very low in every case (as shown in the figure), there is high statistical significance. Some p values that are especially low are bolded for emphasis.

The fact that these correlations are observed, at significant amplitude, in 5 independently generated microarray data sets, sourced using multiple different microarray chips from two different companies, and 1 RNA-seq data set (so a completely different methodology), shows these correlations to be extremely robust and reliable. Moreover these same correlations are observed manifold in further microarray data sets, componentry to the NCI COMPARE [67] rather than the CellMiner [3-4] database, which weren't used to generate this figure. But for which data is presented in the main text.

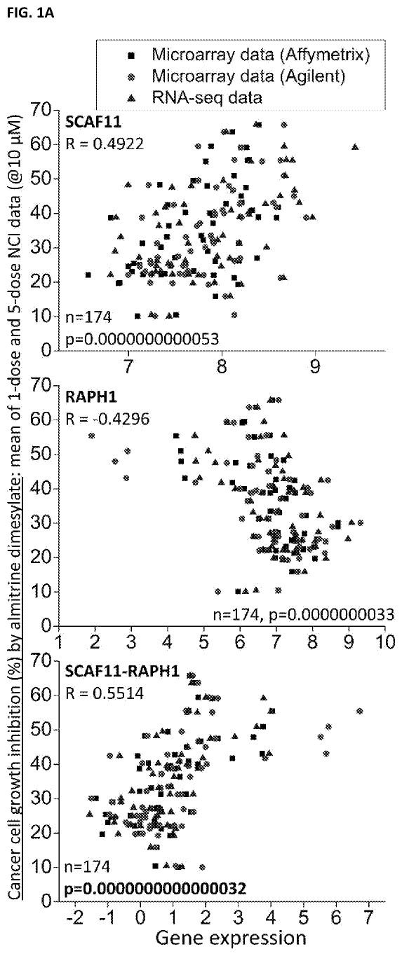

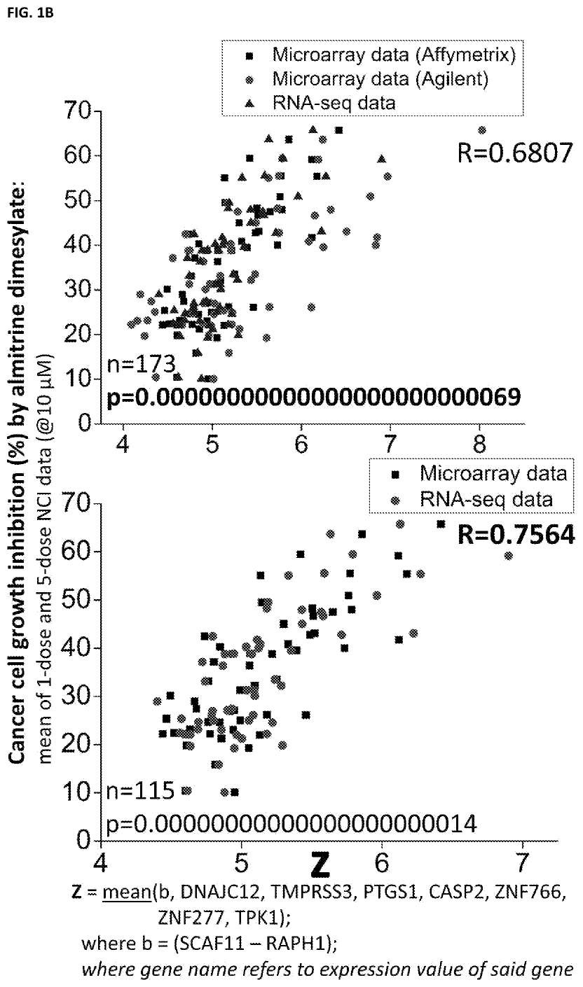

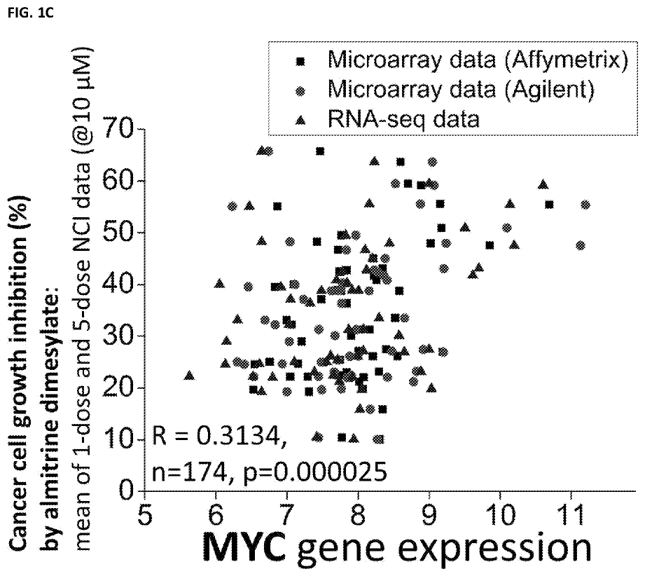

FIG. 1A: Upper panel: Anti-cancer activity of almitrine dimesylate is greater against cancers with greater gene expression of SCAF11. Middle panel: Anti-cancer activity of almitrine dimesylate is less against cancers with greater gene expression of RAPH1. Bottom panel: Anti-cancer activity of almitrine dimesylate is greater against cancers with greater value of SCAF11 gene expression minus RAPH1 gene expression.

FIG. 1B: Anti-cancer activity of almitrine dimesylate is greater with cancers with a greater product of [mean gene expression, of gene(s) that correlates with almitrine dimesylate anti-cancer activity, minus mean gene expression of gene(s) that inversely correlates with almitrine dimesylate anti-cancer activity].

Note, “mean(b, DNAJC12, TMPRSS3, PTGS1, CASP2, ZNF766, ZNF277, TPK1)” refers to (b+DNAJC12+TMPRSS3+PTGS1+CASP2+ZNF766+ZNF277+TPK1)/8, where b=(SCAF11−RAPH1), where gene name refers to the expression value of said gene.

Upper panel shows all the data: Affymetrix and Agilent microarray data, and RNA-seq data. Lower panel omits the Agilent microarray data, which increases the value of R, although there is a decrease in p because of less data points. One can see in the upper panel that the Agilent data (●) has a slightly shallower gradient than both the Affymetrix and RNA-seq data, thence why its omission (lower panel) increases R. In the lower panel, where “microarray data” is referred to, this is only Affymetrix data.

DNAJC12 gene expression data wasn't available in the Agilent microarray data set. So, it wasn't included in its calculation of Z. This is perhaps a contributing factor to the Agilent data having a shallower gradient.

For the Affymetrix microarray data in this sub-figure (only), but not for its Agilent microarray or RNA-seq data, the data point corresponding to the SF-539 cancer cell line is omitted because, when plotted, it is an outlier (not shown). Probably because, distinctly, as can be viewed in the CellMiner database, this cell line is missing from many of the underlying Affymetrix microarray data sets. Thus its mean, taken separately for each gene, is very ill constrained by data, and can more easily be thrown by a wayward data point(s). There is less to average. Thence the power of averaging is diminished. So, the mean data for this cell line is not as robust as the mean data for the other cell lines. So, on this basis alone it should be omitted. Moreover because it is an outlier when plotted. And furthermore, because it has so much less microarray data present than the other cell lines that it has no microarray data at all for one of the genes of interest shown: ZNF766. Note that accordingly, SF-539 was also omitted when calculating the median for calculating the aforementioned independent adjustment/proportionality factors for the Agilent microarray and RNA-seq data in this sub-figure. Interestingly, for the lower panel, without this omission of SF-539 (not shown): R=0.7441, n=116, p=1.06664E-21. So, still very significant.

Different and/or more and/or less gene expressions that correlate, or inversely correlate, with almitrine dimesylate anti-cancer activity (examples of which are disclosed in the main text herein) can be used to make an alternative formula to that shown, but which still conveys predictive power, which can be greater, for how susceptible a cancer is to almitrine dimesylate anti-cancer activity. For non-limiting example, an alternative formula is mean(d, DNAJC12, TMPRSS3, PTGS1, MYC, CBLL1, CASP2, SORL1, SYT1, SUPV3L1, ZNF766, ZNF277, ILF2, HMX1, FABP6, KCNC3, TPK1, CAMKK2, DDX49, LETMD1), where b=(SCAF11-d), where d=mean(ITGB5, RAPH1, RAB23), where gene name refers to the expression value of said gene. For which, only using Affymetrix microarray data (a limitation, but quicker to calculate), and with the SF-539 cell line omitted: R=0.7969=0.8, n=57, p=1.21731E-13 (adding Agilent and RNA-seq data would increase n, whilst R would likely largely hold, decreasing p further).

FIG. 1C: The more a cancer expresses the MYC oncogene, the more likely it is susceptible to the anti-cancer activity of almitrine dimesylate. Wherein for many present cancer drugs this correlation is inverted and greater MYC expression correlates with less anti-cancer drug(s) activity, and poor prognosis. So, almitrine dimesylate disproportionally targets the most dangerous cancers.

### Almitrine and Haematological Cancer

In disclosure embodiments, almitrine and/or a pharmaceutically-acceptable salt, solvate, hydrate or prodrug thereof is administered to, or taken by, a subject, optionally co-administered/taken with a FDA/EMA/MHRA/PMDA/NMPA approved drug(s) (optionally approved for cancer(s)/blood cancer(s), optionally an antileukimic drug(s) such as one or more of imatinib (imatinib mesylate), dasatinib, nilotinib, radotinib, bosutinib, ponatinib, and/or a drug(s) for myeloma such as thalidomide and/or thalidomide analogue(s), optionally incorporated in the same pharmaceutical composition; optionally in co-therapy with radiotherapy, optionally wherein almitrine makes the cancer more radiosensitive/less radioresistant and/or the anti-cancer activities of almitrine and radiotherapy add/synergize, and/or optionally in co-therapy with one or more chemotherapies, optionally wherein almitrine makes the cancer more chemosensitive/less chemoresistant and/or the anti-cancer activities of almitrine and chemotherap[y/ies] add/synergize, and/or the subject undergoes a bone marrow/stem cell transplant), to treat/ameliorate/prevent/combat their hematological/hematopoietic and/or lymphoid cell cancer(s)/immunoproliferative disease(s)/lymphoproliferative disorder(s)/blood cancer(s), and/or cancer(s) of bone marrow and/or lymph system (including their pre-cancers e.g. pre-leukemia), including, without restriction and including all sub-types, one or more of leukemia, lymphoma, myeloma (multiple myeloma)/plasmacytoma/Plasma cell leukemia (PCL), wherein (without restriction) all sub-types under the WHO/French-American-British (FAB)/European Leukaemia Net (ELN) classification system are contemplated, wherein lymphoma (to illustrate and not restrict) can be Hodgkin's Lymphoma (HL), Non-Hodgkin's lymphoma (NHL), an HIV associated lymphoma and/or Epstein-Barr virus-associated lymphoproliferative disease, wherein leukemia can be (to illustrate and not restrict) Acute Leukemia, Chronic Leukemia, or to be more specific: Acute Lymphoblastic Leukemia (ALL), Acute Myeloid Leukemia (AML), Chronic Lymphocytic Leukemia (CLL), Chronic Myeloid Leukemia (CML) {including blast crisis of CML} or a leukemia not necessarily fitting into one of these 4 classifications such as Hairy cell Leukemia (HCL), Hairy Cell Leukemia-Variant (HCL-V), Hairy Cell Leukemia-Japanese Variant (HCL-J), Mixed-Phenotype Acute Leukemia (MPAL, biphenotypic leukaemia, acute biphenotypic ALL, mixture of AML/ALL, the abnormal cells have protein markers for both ALL and AML), aggressive NK-cell leukemia, juvenile myelomonocytic leukemia, large granular lymphocytic leukemia, B cell prolymphocytic leukemia (B-PLL), T-cell prolymphocytic leukemia (T-PLL), Adult T-cell leukemia/lymphoma, T-lymphoblastic leukemia/lymphoma, T-cell acute lymphoblastic leukemia, chronic eosinophilic leukemia (CEL), clonal eosinophilias (may be pre-cancerous or cancerous). Wherein (without restriction), ALL includes precursor B acute lymphoblastic leukemia (B-cell ALL), precursor T acute lymphoblastic leukemia (T-cell ALL), Burkitt's leukemia, Philadelphia chromosome positive ALL, AML includes acute promyelocytic leukemia, acute myeloblastic leukemia, acute megakaryoblastic leukemia, acute erythroid leukemia, acute monocytic leukemia, Acute Myeloid Leukemia with Myelodysplasia Related Changes (AML-MRC), CLL includes B-cell prolymphocytic leukemia, CML includes chronic myelomonocytic leukemia, blast crisis of CML, Philadelphia chromosome positive CML. Wherein (illustrating without restriction) Hodgkin's lymphoma can be classical Hodgkin's lymphoma (nodular sclerosing HL, mixed-cellularity subtype, lymphocyte-rich, lymphocyte depleted), nodular lymphocyte-predominant Hodgkin lymphoma. Non-Hodgkin's lymphoma (NHL) can be (to illustrate and not restrict) follicular lymphoma, primary cutaneous follicular lymphoma, primary cutaneous marginal zone lymphoma (primary cutaneous immunocytoma, marginal zone B-cell lymphoma, mucosa-associated lymphoid tissue lymphoma), intravascular large B-cell lymphoma, Burkitt's lymphoma, Burkitt's-like lymphoma, cutaneous T cell lymphoma, T-cell lymphoma, B-cell lymphoma, diffuse large B cell lymphoma (including primary mediastinal B-cell lymphoma, primary mediastinal (thymic) large B cell lymphoma, Mantle cell lymphoma, germinal center B-cell like diffuse large B-cell lymphoma). Epstein-Barr virus-associated lymphoproliferative disease can be (without restriction) Epstein-Barr virus-positive Hodgkin lymphoma, Epstein-Barr virus-positive (EBV+) diffuse large B cell lymphoma, Epstein-Barr virus-associated diffuse large B cell lymphoma associated with chronic inflammation, Epstein-Barr virus-positive Burkitt lymphoma, Epstein-Barr virus-positive lymphomatoid granulomatosis, Extranodal NK/T-cell lymphoma nasal type, Epstein-Barr virus-associated plasma cell myeloma. Also contemplated by this disclosure is follicular dendritic cell sarcoma (similarities in presentation and markers to lymphoma), Waldenström's macroglobulinemia (WM, type of cancer affecting two types of B cells: lymphoplasmacytoid cells and plasma cells) and Richter's syndrome (RS, wherein CLL or hairy cell leukemia transforms into a Hodgkin or non-Hodgkin lymphoma). Pre-leukemia includes (without restriction) monoclonal B-cell lymphocytosis, transient myeloproliferative disease, myelodysplastic syndrome (MDS), myeloproliferative neoplasm (MPN), myelodysplastic-myeloproliferative disease, lymphoproliferative disorder. Any cancer or pre-cancer mentioned herein is especially contemplated for treatment with almitrine (and/or a pharmaceutically-acceptable salt, solvate, hydrate or prodrug thereof).

### Note

This disclosure encompasses all combinations of aspects of the disclosure noted herein. It is understood that any and all embodiments of the present disclosure may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment. Feature(s) described in connection with one embodiment of the disclosure may be used in conjunction with another embodiment(s), even if not explicitly stated. Any/all of the features described herein (including any accompanying claims, abstract and drawings), and/or all/some of the steps of any method or process so disclosed, may be combined with any of the above aspects in any combination.

### REFERENCES

[1] Oral almitrine to be withdrawn by EU Member States (29 May 2013) http://www.emia.europa.eu/docs/en_GB/document_library/Referrals_document/Almitrine/Position_provided_by_CMDh/WC01S43802.pdf (accessed on 25 Aug. 2021). [2] European Medicines Agency (23 May 2013) Assessment report for almitrine containing medicinal products for oral use.http://www.ema.europa.eu/docs/en_GB/document_library/Referrals_document/Almitrine/Recommendation_provided_by_Pharmacovigilance_Risk_Assessment_Committee/WC500144134.pdf (accessed on 25 Aug. 2021). [3] Reinhold et al. (2012) CellMiner: a web-based suite of genomic and pharmacologic tools to explore transcript and drug patterns in the NCI-60 cell line set. Cancer research. 72(14):3499-511. [4]https://discover.nci.nih.gov/cellminer/home.do (accessed on 25 Aug. 2021), alternatively access: https://dtp.cancer.gov/dtpstandard/dwindex/index.jsp (accessed on 25 Aug. 2021). [5] Salzberg S L (2018) Open questions: How many genes do we have? BMC biology. 16(1):1-3. [6] Paul et al. (1989) Display and analysis of patterns of differential activity of drugs against human tumor cell lines: development of mean graph and COMPARE algorithm. JNCI: Journal of the National Cancer Institute. 81(14):1088-92. [7]https://dtp.cancer.gov/databases_tools/compare.htm (accessed on 25 Aug. 2021). [8] Reinhold et al. (2015) Using CellMiner 1.6 for systems pharmacology and genomic analysis of the NCI-60. Clinical Cancer Research. 21(17):3841-52. [9] Reinhold et al. (2019) RNA sequencing of the NCI-60: Integration into CellMiner and CellMiner CDB. Cancer research. canres-2047. [10] Yu et al. (2012) Up-regulation of human prostaglandin reductase 1 improves the efficacy of hydroxymethylacylfulvene, an antitumor chemotherapeutic agent. Journal of Pharmacology and Experimental Therapeutics. 343(2):426-33. [11] Dick et al. (2004) NADPH alkenal/one oxidoreductase activity determines sensitivity of cancer cells to the chemotherapeutic alkylating agent irofulven. Clinical cancer research. 10(4):1492-9. [12] Tyner J W et al. (2018) Functional genomic landscape of acute myeloid leukaemia. Nature. 562(7728):526. [13] Gholami et al. (2013) Global proteome analysis of the NCI-60 cell line panel. Cell reports. 4(3):609-20. [14] de Kok et al. (2005) Normalization of gene expression measurements in tumor tissues: comparison of 13 endogenous control genes. Laboratory investigation. 85(1):154. [15] Burd et al. (2019) Challenges and approaches to implementing master/basket trials in oncology. Blood advances. 3(14):2237-43. [16] B'chir et al. (1998) Intravenous almitrine bismesylate reversibly induces lactic acidosis and hepatic dysfunction in patients with acute lung injury. Anesthesiology: The Journal of the American Society of Anesthesiologists. 89(4):823-30. [17] Monks et al. (2018) The NCI transcriptional pharmacodynamics workbench: a tool to examine dynamic expression profiling of therapeutic response in the NCI-60 cell line panel. Cancer research. 78(24):6807-6817. [18]Carabet et al. (2019) Therapeutic inhibition of Myc in cancer. Structural bases and computer-aided drug discovery approaches. International journal of molecular sciences. 20(1):120. [19] Shi et al. (2019) Plasma C-MYC level manifesting as an indicator in progression of breast cancer. Biomarkers in medicine. 30(0). [20]Schick et al. (2017) Pathogenesis and therapeutic targeting of aberrant MYC expression in haematological cancers. British journal of haematology. 179(5):724-38. [21] Cortiguera et al. (2015) MYC as therapeutic target in leukemia and lymphoma. Blood and Lymphatic Cancer: Targets and Therapy. 5:75-91. [22] Porro et al. (2011) c-MYC oncoprotein dictates transcriptional profiles of ATP-binding cassette transporter genes in chronic myelogenous leukemia CD34+ hematopoietic progenitor cells. Molecular Cancer Research. 9(8):1054-66. [23] Liu et al. (2012) Myc induced miR-144/451 contributes to the acquired imatinib resistance in chronic myelogenous leukemia cell K562. Biochemical and biophysical research communications. 425(2):368-73. [24] Albajar et al. (2011) MYC in chronic myeloid leukemia: induction of aberrant DNA synthesis and association with poor response to imatinib. Molecular Cancer Research. 9(5):564-76. [25] Gomez-Casares et al. (2013) MYC antagonizes the differentiation induced by imatinib in chronic myeloid leukemia cells through downregulation of p27 KIP1. Oncogene. 32(17):2239. [26] Fang et al. (2009) Transcriptional regulation of survivin by c-Myc in BCR/ABL-transformed cells: implications in anti-leukaemic strategy. Journal of cellular and molecular medicine. 13(8b):2039-52. [27] Sawyers et al. (1992) Dominant negative MYC blocks transformation by ABL oncogenes. Cell. 70(6):901-10. [28] Handa et al. (1997) Bcl-2 and c-myc expression, cell cycle kinetics and apoptosis during the progression of chronic myelogenous leukemia from diagnosis to blastic phase. Leukemia research. 21(6):479-89. [29] Taub et al. (1982) Translocation of the c-myc gene into the immunoglobulin heavy chain locus in human Burkitt lymphoma and murine plasmacytoma cells. Proceedings of the National Academy of Sciences. 79(24):7837-41. [30] Ott et al. (2013) Understanding MYC-driven aggressive B-cell lymphomas: pathogenesis and classification. Blood, The Journal of the American Society of Hematology. 122(24):3884-91. [31] Gomez-Curet et al. (2006) c-Myc inhibition negatively impacts lymphoma growth. Journal of pediatric surgery. 41(1):207-11. [32] Yun et al. (2019) Prognostic significance of MYC oncoprotein expression on survival outcome in patients with acute myeloid leukemia with myelodysplasia related changes (AML-MRC). Leukemia research. 84:106194. [33] Ohanian et al. (2019) MYC protein expression is an important prognostic factor in acute myeloid leukemia. Leukemia & lymphoma. 60(1):37-48. [34] Court et al. (2004) DNA microarray screening of differential gene expression in bone marrow samples from AML, non-AML patients and AML cell lines. Leukemia research. 28(7):743-53. [35] Jones et al. (2010) Ochoa R. Gain of MYC underlies recurrent trisomy of the MYC chromosome in acute promyelocytic leukemia. Journal of Experimental Medicine. 207(12):2581-94. [36] Luo et al. (2005) c-Myc rapidly induces acute myeloid leukemia in mice without evidence of lymphoma-associated antiapoptotic mutations. Blood. 106(7):2452-61. [37] Felsher D W, Bishop J M (1999) Reversible tumorigenesis by MYC in hematopoietic lineages. Molecular cell. 4(2):199-207. [38] Skoda et al. (1995) Expression of c-MYC under the control of GATA-1 regulatory sequences causes erythroleukemia in transgenic mice. Journal of Experimental Medicine. 181(5):1603-13. [39] Pan et al. (2014) Inhibition of c-Myc overcomes cytotoxic drug resistance in acute myeloid leukemia cells by promoting differentiation. PLoS One. 9(8):e105381. [40] Holt et al. (1988) An oligomer complementary to c-myc mRNA inhibits proliferation of HL-60 promyelocytic cells and induces differentiation. Molecular and cellular biology. 8(2):963-73. [41] Brondfield et al. (2015) Direct and indirect targeting of MYC to treat acute myeloid leukemia. Cancer chemotherapy and pharmacology. 76(1):35-46. [42]Huang et al. (2006) A small-molecule c-Myc inhibitor, 10058-F4, induces cell-cycle arrest, apoptosis, and myeloid differentiation of human acute myeloid leukemia. Experimental hematology. 34(11):1480-9. [43]Zhang et al. (2011) Differential requirements for c-Myc in chronic hematopoietic hyperplasia and acute hematopoietic malignancies in Pten-null mice. Leukemia. 25(12):1857. [44] Li et al. (2014) SIRT1 activation by a c-MYC oncogenic network promotes the maintenance and drug resistance of human FLT3-ITD acute myeloid leukemia stem cells. Cell stem cell. 15(4):431-46. [45] Rayeroux K C, Campbell L J (2009) Gene amplification in myeloid leukemias elucidated by fluorescence in situ hybridization. Cancer genetics and cytogenetics. 193(1):44-53. [46] Thomas et al. (2004) Double minute chromosomes in monoblastic (M5) and myeloblastic (M2) acute myeloid leukemia: two case reports and a review of literature. American journal of hematology. 77(1):55-61. [47] Borga C, Frazer J K (2019) Zebrafish MYC-induced leukemia models: unique in vivo systems to study B and T cell acute lymphoblastic leukemia. International Journal of Hematologic oncology. 8(1). [48] Roderick et al. (2014) c-Myc inhibition prevents leukemia initiation in mice and impairs the growth of relapsed and induction failure pediatric T-ALL cells. Blood. 123(7):1040-50. [49] Malempati et al. (2006) Aberrant stabilization of c-Myc protein in some lymphoblastic leukemias. Leukemia. 20(9):1572. [50] Sharma et al. (2006) Notch1 contributes to mouse T-cell leukemia by directly inducing the expression of c-myc. Molecular and cellular biology. 26(21):8022-31. [51] Sharma et al. (2007) The Notch1/c-Myc pathway in T cell leukemia. Cell cycle. 6(8):927-30. [52] Palomero et al. (2006) NOTCH1 directly regulates c-MYC and activates a feed-forward-loop transcriptional network promoting leukemic cell growth. PNAS. 103(48):18261-6. [53] Bonnet et al. (2011) Posttranscriptional deregulation of MYC via PTEN constitutes a major alternative pathway of MYC activation in T-cell acute lymphoblastic leukemia. Blood. 117(24):6650-9. [54] Chiang et al. (2016) High selective pressure for Notch1 mutations that induce Myc in T-cell acute lymphoblastic leukemia. Blood. 128(18):2229-40. [55] Weng et al. (2006) c-Myc is an important direct target of Notch1 in T-cell acute lymphoblastic leukemia/lymphoma. Genes & development. 20(15):2096-109. [56]Zhan et al. (2002) Global gene expression profiling of multiple myeloma, monoclonal gammopathy of undetermined significance, and normal bone marrow plasma cells. Blood. 99(5):1745-57. [57] De Vos et al. (2002) Comparison of gene expression profiling between malignant and normal plasma cells with oligonucleotide arrays. Oncogene. 21(44):6848. [58] Chappell et al. (2000) A mutation in the c-myc-IRES leads to enhanced internal ribosome entry in multiple myeloma: a novel mechanism of oncogene de-regulation. Oncogene. 19(38):4437. [59] Chiecchio et al. (2009) Frequent upregulation of MYC in plasma cell leukemia. Genes, Chromosomes and Cancer. 48(7):624-36. [60] Glitza et al. (2015) Chromosome 8q24. 1/c-MYC abnormality: a marker for high-risk myeloma. Leukemia & lymphoma. 56(3):602-7. [61] Chesi et al. (2008) AID-dependent activation of a MYC transgene induces multiple myeloma in a conditional mouse model of post-germinal center malignancies. Cancer cell. 13(2):167-80. [62] Affer et al. (2014) Promiscuous MYC locus rearrangements hijack enhancers but mostly super-enhancers to dysregulate MYC expression in multiple myeloma. Leukemia. 28(8):1725. [63] Chng et al. (2011) Clinical and biological implications of MYC activation: a common difference between MGUS and newly diagnosed multiple myeloma. Leukemia. 25(6):1026. [64] Holien T, Sundan A (2012) Oncogene addiction to c-MYC in myeloma cells. Oncotarget. 3(8):739. [65] Kuehl W M, Bergsagel P L (2012) MYC addiction: a potential therapeutic target in MM. Blood. 120(12):2351-2. [66] Holien et al. (2012) Addiction to c-MYC in multiple myeloma. Blood. 120(12):2450-3. [67] Shaffer et al. (2008) IRF4 addiction in multiple myeloma. Nature. 454(7201):226. [68] Sun et al. (2015) The nucleolar ubiquitin-specific protease USP36 deubiquitinates and stabilizes c-Myc. Proceedings of the National Academy of Sciences. 112(12):3734-9. [69] David et al. (2010) HnRNP proteins controlled by c-Myc deregulate pyruvate kinase mRNA splicing in cancer. Nature. 463(7279):364. [70] Gopisetty et al. (2013) Epigenetic regulation of CD133/PROM1 expression in glioma stem cells by Sp1/myc and promoter methylation. Oncogene. 32(26):3119. [71] Bertolini et al. (2009) Highly tumorigenic lung cancer CD133+ cells display stem-like features and are spared by cisplatin treatment. Proceedings of the National Academy of Sciences. 106(38):16281-6. [72] Tolba et al. (2013) Expression of CD133 in acute leukemia. Medical Oncology. 30(2):527. [73] Li et al. (2019) High expression of SLC38A1 predicts poor prognosis in patients with de novo acute myeloid leukemia. Journal of cellular physiology. 234(11):20322-8. [74] Zhang et al. (2099) Predictors of primary imatinib resistance in chronic myelogenous leukemia are distinct from those in secondary imatinib resistance. Journal of clinical oncology. 27(22):3642. [75] Penfold et al. (2018) CAMKK2 promotes prostate cancer independently of AMPK via increased lipogenesis. Cancer research. 78(24):6747-61. [76] Hui et al. (2019) CBLL1 is highly expressed in non-small cell lung cancer and promotes cell proliferation and invasion. Thoracic cancer. [77] Uno et al. (2019) Increased expression of DNAJC12 is associated with aggressive phenotype of gastric cancer. Annals of surgical oncology. 26(3):836-44. [78] Ohmachi et al. (2006) Fatty acid binding protein 6 is overexpressed in colorectal cancer. Clinical cancer research. 12(17):5090-5. [79] Jin et al. (2018) Interleukin enhancer binding factor 2 is a prognostic biomarker for breast cancer that also predicts neoadjuvant chemotherapy responses. American journal of translational research. 10(6):1677. [80]Chowdhry et al. (2019) NAD metabolic dependency in cancer is shaped by gene amplification and enhancer remodelling. Nature. 569(7757):570. [81] Xu et al. (2019) NPTX2 promotes colorectal cancer growth and liver metastasis by the activation of the canonical Wnt/β-catenin pathway via FZD6. Cell death & disease. 10(3):217. [82] Zhou et al. (2018) Pir2/Rnf144b is a potential endometrial cancer biomarker that promotes cell proliferation. Cell death & disease. 9(5):504. [83] Rui et al. (2015) TMPRSS3 is a novel poor prognostic factor for breast cancer. International journal of clinical and experimental pathology. 8(5):5435. [84] Liu et al. (2019) ZnF277 regulates ovarian cancer cell proliferation and invasion through inhibition of PTen. OncoTargets and therapy. 12:3031. [85] Jonus et al. (2018) The adaptive regulation of thiamine pyrophosphokinase-1 facilitates malignant growth during supplemental thiamine conditions. Oncotarget. 9(83):35422. [86] Hua et al. (2018) Downregulation of Annexin All (ANXA11) inhibits cell proliferation, invasion, and migration via the AKT/GSK-3p pathway in gastric cancer. Medical science monitor: international medical journal of experimental and clinical research. 24:149. [87] Abudureheman et al. (2018) High MLL2 expression predicts poor prognosis and promotes tumor progression by inducing EMT in esophageal squamous cell carcinoma. Journal of cancer research and clinical oncology. 144(6):1025-35. [88] Chen et al. (2017) MLL2, not MLL1, plays a major role in sustaining MLL-rearranged acute myeloid leukemia. Cancer cell. 31(6):755-70.

1. A method to predict (give an indication of; give a probabilistic indication of) the susceptibility/responsiveness of a subject's cancer to almitrine (and/or a pharmaceutically-acceptable salt, solvate, hydrate or prodrug thereof) administration, comprising the steps of:
i) obtaining (or starting with) a biological sample(s) from the subject;
ii) ex vivo/in vitro, determining the mRNA and/or cDNA and/or protein expression level of one or more of SCAF11, RAPH1, DNAJC12, TMPRSS3, PTGS1, CASP2, ZNF766, ZNF277, TPK1, MYC, CBLL1, SORL1, SYT1, SUPV3L1, ILF2, HMX1, FABP6, KCNC3, CAMKK2, DDX49, LETMD1, ITGB5, RAB23 in the sample(s) obtained from the subject to obtain a value, or values, representing this level(s); and
iii) comparing said expression level(s) to a reference expression level(s):
comparing the value, or values, of the level(s) from step (ii) with a standard/reference value, or a set of standard/reference values, wherein this comparison predicts (gives an indication of; gives a probabilistic indication of) the subject's cancer's susceptibility/responsiveness to almitrine (and/or a pharmaceutically-acceptable salt, solvate, hydrate or prodrug thereof) administration.

2. The method of claim 1 wherein a higher level of one or more of SCAF11, DNAJC12, TMPRSS3, PTGS1, CASP2, ZNF766, ZNF277, TPK1, MYC, CBLL1, SORL1, SYT1, SUPV3L1, ILF2, HMX1, FABP6, KCNC3, CAMKK2, DDX49, LETMD1 (any one or more of which can be termed “GENE Q” in this claim set) protein(s) and/or nucleic acid(s) in the sample, and/or a lower level of one or more of RAPH1, ITGB5, RAB23 (any one or more of which can be termed “GENE Y” in this claim set) protein(s) and/or nucleic acid(s) in the sample, relative to the standard/reference value, or a set of standard/reference values, predicts (gives an indication of; gives a probabilistic indication of) [optionally greater] susceptibility/responsiveness of the subject's cancer to treatment with almitrine (and/or a pharmaceutically-acceptable salt, solvate, hydrate or prodrug thereof).

3. The method of claim 1 wherein a higher level of SCAF11 protein(s) and/or nucleic acid(s) in the sample, and/or a lower level of RAPH1 protein(s) and/or nucleic acid(s) in the sample, relative to the standard/reference values, preferably such that the differential between SCAF11 and RAPH1 expression is greater than the corresponding standard/reference differential value(s), predicts (gives an indication of; gives a probabilistic indication of) [optionally greater] susceptibility/responsiveness of the subject's cancer to treatment with almitrine (and/or a pharmaceutically-acceptable salt, solvate, hydrate or prodrug thereof).

4. The method of claim 1 wherein a higher level of MYC protein(s) and/or nucleic acid(s) in the sample, relative to the standard/reference value(s), predicts (gives an indication of; gives a probabilistic indication of) [optionally greater] susceptibility/responsiveness of the subject's cancer to treatment with almitrine (and/or a pharmaceutically-acceptable salt, solvate, hydrate or prodrug thereof).

5. The method of claim 1, wherein the determination of
(a) a higher level of “GENE Q” protein(s) and/or “GENE Q” nucleic acid(s), and/or
(b) a lower level of “GENE Y” protein(s) and/or “GENE Y” nucleic acid(s),
in said sample obtained from the subject is carried out by comparing the measured “GENE Q” and/or “GENE Y” protein(s) and/or nucleic acid(s) level(s) in said sample
i) relative to a standard value, or a set of standard values, of the level of “GENE Q” and/or “GENE Y” protein(s) and/or nucleic acid(s) from a biological sample(s) from another subject(s) with the same cancer (e.g. same cancer histotype) as the subject; and/or
ii) relative to a standard value, or a set of standard values, of the level of “GENE Q” and/or “GENE Y” protein(s) and/or nucleic acid(s) from one or more cancer cell lines, optionally one or more cancer cell lines derived from the same tissue type as the subject's cancer; and/or
iii) relative to a standard value, or a set of standard values, of the level of “GENE Q” and/or “GENE Y” protein(s) and/or nucleic acid(s) from a sample, or samples, from normal cell(s)/tissue(s); and/or
iv) relative to a standard value, or a set of standard values, of the level of “GENE Q” and/or “GENE Y” protein(s) and/or nucleic acid(s) from a sample, or samples, obtained from the same subject before any initiation of almitrine (and/or a pharmaceutically-acceptable salt, solvate, hydrate or prodrug thereof) administration;
preferably wherein each “GENE Q” assayed in the biological sample(s) from the subject is compared to the same “GENE Q” type in the standard value(s);
preferably wherein each “GENE Y” assayed in the biological sample(s) from the subject is compared to the same “GENE Y” type in the standard value(s).

6. A microarray chip comprising/containing a nucleic acid probe(s) for (i.e. a single-stranded nucleotide sequence(s) that is complimentary for/hybridisable to, preferably with ≥85% sequence identity to) mRNA and/or cDNA (and/or part(s) thereof, e.g. ≥15 consecutive nucleotides thereof) of one or more of SCAF11, RAPH1, DNAJC12, TMPRSS3, PTGS1, CASP2, ZNF766, ZNF277, TPK1, MYC, CBLL1, SORL1, SYT1, SUPV3L1, ILF2, HMX1, FABP6, KCNC3, CAMKK2, DDX49, LETMD1, ITGB5, RAB23.

7. A method of using the microarray chip of claim 6 comprising contacting it with a sample(s) from the subject, measuring the hybridization, to predict/diagnose if cancer in the subject is likely to be susceptible/responsive to the anti-cancer activity of almitrine (and/or a pharmaceutically-acceptable salt, solvate, hydrate or prodrug thereof) administration.

8. The method of any one or more of claims 1-7, wherein a subject(s) whose cancer has
(a) a higher “GENE Q” protein(s) and/or “GENE Q” nucleic acid(s) amount, above a threshold amount, and/or
(b) a lower “GENE Y” protein(s) and/or “GENE Y” nucleic acid(s) amount, below a threshold amount, is predicted (giving an indication of; giving a probabilistic indication of) to be helped/treated by almitrine (and/or a pharmaceutically-acceptable salt, solvate, hydrate or prodrug thereof) administration for anti-cancer therapy,
and/or a subject(s) whose cancer has
(c) a lower “GENE Q” protein(s) and/or “GENE Q” nucleic acid(s) amount, below the threshold amount, and/or
(d) a higher “GENE Y” protein(s) and/or “GENE Y” nucleic acid(s) amount, above the threshold amount, is not.

9. The method of any one or more of claims 1-8, wherein a subject(s) whose cancer has
(a) a higher “GENE Q” protein(s) and/or “GENE Q” nucleic acid(s) amount, above a threshold amount, and/or
(b) a lower “GENE Y” protein(s) and/or “GENE Y” nucleic acid(s) amount, below a threshold amount, is administered a therapeutically effective amount of almitrine (and/or a pharmaceutically-acceptable salt, solvate, hydrate or prodrug thereof),
and/or a subject(s) whose cancer has
(c) a lower “GENE Q” protein(s) and/or “GENE Q” nucleic acid(s) amount, below the threshold amount, and/or
(d) a higher “GENE Y” protein(s) and/or “GENE Y” nucleic acid(s) amount, above the threshold amount, is not (optionally wherein they are administered with a different cancer treatment(s)/drug(s) instead).

10. The method of claim 1, wherein by ranking the level of one or more of “GENE Q” and/or “GENE Y” protein(s) and/or nucleic acid(s) in biological samples from different subjects with cancer, preferably with cancer of same/similar type and/or with shared feature(s), then one can rank which of these cancers (those with most “GENE Q”, and/or least “GENE Y”, mRNA/cDNA/protein, and/or those with most differential between the amount of “GENE Q” and “GENE Y” mRNA/cDNA/protein) are most likely to be susceptible/responsive to anti-cancer treatment with almitrine (and/or a pharmaceutically acceptable salt, solvate, hydrate or prodrug thereof).

11. The method of any one or more of claims 1-10, wherein
the threshold amount for “GENE Q” is the mean/median/mode/decile/quartile/percentile, or some other function, of “GENE Q” protein(s) and/or “GENE Q” nucleic acid(s) amount in a normal and/or cancer derived sample(s) cohort; and/or
the threshold amount for “GENE Y” is the mean/median/mode/decile/quartile/percentile, or some other function, of “GENE Y” protein(s) and/or “GENE Y” nucleic acid(s) amount in a normal and/or cancer derived sample(s) cohort.

12. The method of any one or more of claims 1-11, wherein
the amount of “GENE Q” in the biological sample(s) from the subject (e.g. cancer sample) is normalized against the amount of one or more reference gene products in this biological sample(s) to obtain a normalized expression level of “GENE Q”, and wherein comparison is made against a standard value, or a set of standard values, that have been normalized by the same method; and/or
the amount of “GENE Y” in the biological sample(s) from the subject (e.g. cancer sample) is normalized against the amount of one or more reference gene products in the biological sample(s) to obtain a normalized expression level of “GENE Y”, and wherein comparison is made against a standard value, or a set of standard values, that have been normalized by the same method.

13. The method according to claim 1, wherein the sample(s) is derived from one or more of “liquid biopsy”, body wash (e.g. a lung wash sample), bodily fluid(s), tissue(s), normal tissue(s), cancer tissue(s), suspected cancer tissue(s), circulating cancer cells, cell line(s), urine, feces, plasma, serum and/or whole blood, or an extract or processed sample produced from any thereof.

14. The method of claim 1 wherein the sample comprises/contains cancer cells, and/or part(s) thereof, and/or nucleic acid and/or protein molecules thereof, from the subject's cancer/tumour.

15. The method of claim 1 wherein the subject is human, optionally elderly (e.g. >60 years old).

16. The method of claim 1 with the added step of generating an oral/written report (optionally accessible on a computer/phone/electronic device, optionally transmitted over the internet/intranet, optionally transmitted by electromagnetic radiation) based on the information, optionally information sourced from conducting the method of claim 1 more than one time.

17. A method of requesting/instructing for the method of claim 1, and optionally claim 16, to be performed for a subject with cancer, and using the output to decide/determine (and/or as an input into deciding/determining) whether to administer almitrine (and/or a pharmaceutically-acceptable salt, solvate, hydrate or prodrug thereof) to this subject.

18. A method of requesting/instructing for the method of claim 1, and optionally claim 16, to be performed for a subject with cancer, and later administering a therapeutically effective amount of almitrine (and/or a pharmaceutically-acceptable salt, solvate, hydrate or prodrug thereof) to this subject.

19. A method of predicting (giving an indication of; giving a probabilistic indication of) whether a cancer, optionally inside a subject, is susceptible/responsive to almitrine (and/or a pharmaceutically-acceptable salt, solvate, hydrate or prodrug thereof) treatment by comparing whether its gene/mRNA/cDNA/protein expression of one or more of “GENE Q” is equal or higher (and/or substantially similar), and/or its gene/mRNA/cDNA/protein expression of one or more of “GENE Y” is equal or lower (and/or substantially similar), than a cancer(s) (optionally a cancer cell line[s]) known to be susceptible/responsive to almitrine (and/or a pharmaceutically-acceptable salt, solvate, hydrate or prodrug thereof) administration.

20. The method of claim 19 wherein if a subject's cancer is predicted to be susceptible/responsive to almitrine (and/or a pharmaceutically-acceptable salt, solvate, hydrate or prodrug thereof) this subject is administered with a therapeutically effective amount of almitrine (and/or a pharmaceutically-acceptable salt, solvate, hydrate or prodrug thereof).

21. A method of treating a subject (e.g. a human subject) diagnosed with cancer comprising administering a therapeutically effective amount of almitrine (and/or a pharmaceutically-acceptable salt, solvate, hydrate or prodrug thereof) to the subject with a difference score determined from a cancer/tumor sample (optionally from a biopsy) from the subject;
wherein the difference score is substantially similar to a difference score of a subject's cancer (e.g. a human subject) known to be sensitive/responsive to almitrine (and/or a pharmaceutically-acceptable salt, solvate, hydrate or prodrug thereof) administration; and/or
wherein the difference score is substantially dissimilar to a difference score of a subject's cancer (e.g. a human subject) known to be resistant to almitrine (and/or a pharmaceutically-acceptable salt, solvate, hydrate or prodrug thereof) administration; and
wherein the difference score is the difference between a level of expression of one or more “GENE Q” (if more than one “GENE Q”, their mean is taken and used) and a level of expression of one or more “GENE Y” (if more than one “GENE Y”, their mean is taken and used);
optionally wherein the difference score is the difference between the level of expression of SCAF11 and the level of expression of RAPH1.

22. A method of treating a subject (e.g. a human subject) diagnosed with cancer comprising administering a therapeutically effective amount of almitrine (and/or a pharmaceutically-acceptable salt, solvate, hydrate or prodrug thereof) to the subject, wherein the subject's cancer has been predicted/determined to be responsive to almitrine (and/or a pharmaceutically-acceptable salt, solvate, hydrate or prodrug thereof) administration according to a method comprising:
(a) contacting a cancer/tumor sample (optionally from a biopsy) from the subject, comprising nucleic acid molecules, with a device (optionally a microarray) comprising:
(i) single-stranded nucleic acid molecules (optionally immobilized on a solid substrate) capable of specifically hybridizing with the nucleotides (e.g. mRNA and/or cDNA) of one or more of “GENE Q”; and
(ii) single-stranded nucleic acid molecules (optionally immobilized on a solid substrate) capable of specifically hybridizing with the nucleotides (e.g. mRNA and/or cDNA) of one or more of “GENE Y”; and
(b) detecting a level of expression of one or more of “GENE Q” and one or more of “GENE Y”; measuring hybridization between the one or more nucleic acid molecules from the sample and the single-stranded nucleic acid molecules of the device to detect a level of expression of one or more of “GENE Q” and one or more of “GENE Y”; and
(c) calculating a difference score for the subject's cancer by subtracting the level of expression of one or more of “GENE Y” (if more than one “GENE Y”, their mean is taken and used) from the level of expression of one or more of “GENE Q” (if more than one “GENE Q”, their mean is taken and used);
wherein the difference score is substantially similar to the difference score of a subject's cancer (e.g. a human subject) known to be sensitive/responsive to almitrine (and/or a pharmaceutically-acceptable salt, solvate, hydrate or prodrug thereof) administration; and/or
wherein the difference score is substantially dissimilar to the difference score of a subject's cancer (e.g. a human subject) known to be resistant to almitrine (and/or a pharmaceutically-acceptable salt, solvate, hydrate or prodrug thereof) administration;
optionally wherein the difference score is the difference between the level of expression of SCAF11 and the level of expression of RAPH1.

23. A method of treating a subject (e.g. a human subject) diagnosed with cancer, wherein their cancer has high/overexpressed MYC protein and/or nucleic acid expression, optionally wherein this greater MYC amount confers (or is at least associated/correlated with its) resistance (or decreased susceptibility) to a cancer treatment(s)/drug(s) {non-limiting e.g. imatinib/dasatinib/nilotinib/radotinib/bosutinib/ponatinib, or a salt thereof}, optionally wherein the cancer is AML or CML or NSCLC, comprising administering a therapeutically effective amount of almitrine (and/or a pharmaceutically-acceptable salt, solvate, hydrate or prodrug thereof) to the subject, optionally in co-therapy with the aforementioned cancer treatment(s)/drug(s) in this claim, optionally conferring synergistic anti-cancer activity.
